(19) United States
(12) Patent Application Publication
(10) Pub. No.: US 2006/0128613 A1
(43) Pub. Date: Jun. 15, 2006

(54) Melanocortin receptor ligands

(75) Inventors: Frank Hallock Ebetino, Cincinnati, OH (US); Adam Wieslaw Mazur, Mason, OH (US); Jeffrey Charles Hayes, West Chester, OH (US); Feng Wang, Cincinnati, OH (US); Mark Gregory Solinsky, Cincinnati, OH (US); Anny-Odile Colson, Loveland, OH (US); Qishen Lin, San Diego, CA (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/386,962

(22) Filed: Mar. 12, 2003

(51) Int. Cl. A61K 38/12; C07K 7/64

(52) U.S. Cl. 514/9; 530/317

(57) ABSTRACT

Disclosed are MC-4 and/or MC-3 receptor ligands, the ligands having a structure according to Formula (I): 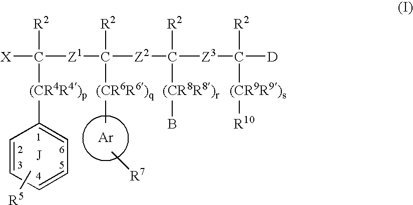
wherein R², R⁴, R^{4′}, R⁵, R⁶, R^{6′}, R⁷, R⁸, R^{8′}, R⁹, R^{9′}, R¹⁰, Ar, Z¹, Z², Z³, X, B, D, p, q, r and s are as described in the specification and claims, and optical isomers, diastereomers or enantiomers thereof; pharmaceutically-acceptable salts, hydrates, and biohydrolyzable esters, amides or imides thereof. Also disclosed are pharmaceutical compositions comprising the ligands of Formula (I), as well as methods of treating diseases mediate by the MC-4/MC-3 receptors, as described in the Detailed Descriptions section of the specification.

### CROSS REFERENCE TO RELATED APPLICATION

This application claims the benefit of International Appliction No. PCT/US01/30051, filed Sep. 26, 2001 which claims benefit of U.S. Provisional Application No. 60/235,858, filed Sep. 27, 2000.

### FIELD OF THE INVENTION

The present invention relates to new melanocortin (MC) receptor ligands. These ligands preferably exhibit selectivity for the MC-4 and/or the MC-3 receptors relative to the other melanocortin receptors (in particular the MC-1 receptor).

### BACKGROUND OF THE INVENTION

Melanocortin peptides (melanocortins) are natural peptide hormones in animals and man that bind to and stimulate MC receptors. Examples of metanocortins are α-MSH (melanocyte stimulating hormone), β-MSH, γ-MSH, ACTH (adrenocorticotropic hormone) and their peptide fragments. MSH is mainly known for its ability to regulate peripheral pigmentation (Eberle 1988), whereas ACTH is known to induce steroidoneogenesis (Simpson and Waterman, 1988). The melanocortin peptides also mediate a number of other physiological effects. They are reported to affect motivation, learning, memory, behavior, inflammation, body temperature, pain perception, blood pressure, heart rate, vascular tone, natriuresis, brain blood flow, nerve growth and repair, placental development, aldosterone synthesis and release, thyroxin release, spermatogenesis, ovarian weight, prolactin and FSH secretion, uterine bleeding in women, sebum and pheromone secretion, sexual activity, penile erection, blood glucose levels, intrauterine fetal growth, food motivated behavior, as well as other events related to parturition.

ACTH and the various MSH peptides share the tetrapeptide core His-Phe-Arg-Trp. All of the peptides are derived from the proteolytic processing of the pro-peptide pre-opiomelanocortin (POMC). In the past several years, five distinct melanocortin receptor subtypes have been identified. These MC receptors belong to the class of 7 transmembrane domain G-protein coupled receptors. The five MC receptors, termed MC-1, MC-2, MC-3, MC-4 and MC-5, all couple in a stimulatory fashion to cAMP. Of these, the MC-2 receptor is the ACTH receptor, whereas the others constitute subtypes of MSH receptors. The MC-1 receptor is present on melanocytes and melanoma. The MC-2 receptor is present predominantly in the adrenal gland. The mRNA for the MC-3 receptor has been found in the brain, as well as in placental and gut tissues (Gantz et al. 1993a, Desarnaud et al. 1994, Roselli Rehfuss et al. 1993). The MC-4 receptor has been found primarily in the brain (Gantz et al. 1993b; Mountjoy et al 1994). The MC-5 receptor is expressed in the brain, as well as in several peripheral tissues (Chhajlani et al 1993; Gantz et al 1994; Griffon et al 1994; Labbu et al. 1994; Barrett et al. 1994; Fathi et al. 1995). More recent data from humans indicate that all of the cloned MC-receptors have a wider tissue distribution (Chhajlani, 1996) than originally thought.

As discussed above, the members of the melanocortin receptor family can be differentiated on the basis of their tissue distribution. Both the MC-4 and MC-3 receptors have been localized to the hypothalamus, a region of the brain believed to be involved in the modulation of feeding behavior. Compounds showing selectivity for the MC-4/MC-3 receptors have been shown to alter food intake following intracerebroventricular and peripheral injection in rodents. Specifically, agonists have been shown to reduce feeding, while antagonists have been shown to increase feeding. See, Fan, W. et al., “Role of Melanocortinergic Neurons in Feeding and the Agouti Obesity Syndrome”, Nature, 385(6612), pp. 165-8 (Jan. 9, 1997).

The role of the MC-4 receptor subtype has been more clearly defined in the control of eating and body weight regulation in mammals. See, e.g., Huszer, D. et al., “Targeted Disruption of the Melanocortin-4 Receptor Results in Obesity in Mice”, Cell, Vol. 88, pp. 131-141 (1997); Klebig, M. L. et al., “Ectopic Expression of the Agouti Gene in Transgenic Mice Causes Obesity, Features of Type II Diabetes, and Yellow Fur”, Proc. Natl Acad Sci., Vol. 92, pp. 4728-32 (1995); Karbon, W. et al., “Expression and Function of Argt, a Novel Gene Related to Agouti”, Abstract from the Nineteenth Annual Winter Neuropeptide Conference (1998); Fan, W. et al., “Role of Melanocortinergic Neurons in Feeding and the Agouti Obesity Syndrome”, Nature, Vol. 385, pp. 165-168 (1997); Seely, R. J., “Melanocortin Receptors in Leptin Effects”, Nature, Vol. 390, p. 349 (1997); Comuzzie, A. G., “A Major Quantitative Trait Locus Determining Serum Leptin Levels and Fat Mass is Located on Human Chromosome 2”, Nat. Gen., Vol. 15, pp. 273-276 (1997); Chagnon, Y. C. et al., “Linkage and Association Studies Between the Melanocortin Receptors 4 and 5 Genes and Obesity-Related Phenotypes in the Quebec Family Study”, Mol. Med., Vol 3(10), pp. 663-673 (1997); Lee, F. and Huszar, D, “Screening Methods for Compounds Useful in the Regulation of Body Weight”, World Patent Publication WO 97/47316 (1997); and Shutter, J. R. et al., “Hypothalamic Expression of ART, a Novel Gene Related to Agouti, is Up-Regulated in Obese and Diabetic Mutant Mice”, Gen. & Dev. Vol. 11, pp. 593-602 (1997). Stimulation of the MC-4 receptor by its endogenous ligand, αMSH, produces a satiety signal and may be the downstream mediator of the leptin satiety signal. It is believed that by providing potent MC-4 receptor agonists, appetite may be suppressed and weight loss benefits may be achieved.

The role of the MC3 receptor subtype has recently been defined in the control of body weight regulation and energy partitioning. See, e.g., Chen, A. S. et al., “Inactivation of the mouse melanocortin-3 receptor results in increased fat mass and reduced lean body mass”, Nature Genetics, Vol 26, pp 97-102 (2000); Butler, A. A. et al., “A Unique Metabolic Syndrome Causes Obesity in the Melanocortin-3 Receptor—Deficient Mouse”, Endocrinology, Vol 141, pp 3518-3521 (2000). It is believed that MC-3 receptor agonists may modulate energy partitioning and is weight loss benefits may be achieved.

These studies imply a non-redundant role for the MC-3 receptor, compared to MC-4 receptor, in energy homeostasis. Therefore, compounds that stimulate both MC-3 and MC-4 receptors may enhance the weight loss benefits compared to compounds that are selective for either the MC-3 or MC-4 receptor subtypes.

The Applicants have discovered a class of compounds that surprisingly have high affinity for the MC-4 and/or the MC-3 receptor subtypes, and that are typically selective for these MC receptors relative to the other melanocortin receptor subtypes, particularly the MC-1 subtype. It is therefore an object of this invention to provide compounds that have affinity for the MC-4 and/or the MC-3 receptor subtypes. It is a further object of the invention to provide means for administration of said compounds to animals or man. Still other objects of the invention will be evident from the following disclosure of the invention.

### DISCLOSURE OF THE INVENTION

The invention relates to a class of compounds that are ligands for receptors of the MC-4 and/or the MC-3 subtype. In particular, the invention relates to a compound having a structure according to Formula (I): 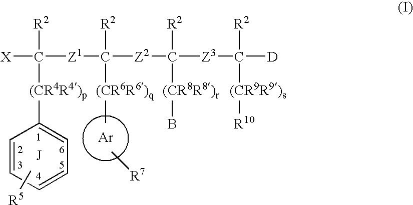
wherein
(A) X is selected from hydrogen, fluoro, aryloxy, acyloxy, OR¹, SR¹, —NR¹R^{1′} and —CHR¹R^{1′}, where R¹ and R^{1′} are independently selected from the group consisting of hydrogen, alkyl and acyl;
(B) (1) each R² is independently selected from the group consisting of hydrogen, alkyl halo and heteroalkyl; or
   (2)(a) two consecutive R² moieties, or consecutive R² and R³ moieties, may join to form a 3 to 8 membered carbocyclic or heterocyclic ring; or
      (b) the R² bonded to the carbon atom that is bonded to X and Z¹ and an R⁵ moiety can optionally join to form a carbocyclic or heterocyclic ring that is fused to phenyl ring J; or
      (c) the R² bonded to the carbon atom that is bonded to ring Ar can join with an R⁷ to form a ring fused to ring Ar; or
      (d) the R² bonded to the carbon atom that is bonded to Z² and Z³ can optionally join with R⁸ to form a carbocyclic or heterocyclic ring; or
      (e) the R² bonded to the carbon atom that is bonded to Z³ and D can optionally join with R¹⁰ to form a carbocyclic or heterocyclic ring;
(C) each of Z¹, Z² and Z³ is independently selected from —OC(R³)(R^{3a})—; —C(R³)(R^{3a})O—; —S(O)ₐC(R³)(R^{3a})—, where a is 0, 1 or 2; —C(R³)(R^{3a})S(O)_{b}—, where b is 0, 1 or 2; —N(R^{3c})C(R³)(R^{3a}); —C(R³)(R^{3a})N(R^{3c})—; —C(O)N(R^{3d})—; N(R^{3d})C(O)—; —C(O)C(R³)(R^{3a}); —C(R³)(R^{3a})C(O)—; C(R³)(R^{3a})C(R^{3b})(R^{3c})—; —C(R³)═C(R^{3a})—; —C≡C—; —SO₂N(R^{3d})—; —N(R^{3d})SO₂—; —C(R³)(R^{3a})P(═O)(OR^{3f})—; —P(═O)(OR^{3f})C(R³)(R^{3a})—; —N(R^{3d})P(═O)(OR^{3f})—; —P(═O)(O^{3f})N(R^{3d})—; —P(═O)(OR^{3f})O—; —O—P(═O)(OR^{3f})—; a cycloalkyl having from 3 to 8 ring atoms and a heterocycloalkyl having from 4 to 8 ring atoms; wherein
   (1) each of R³, R^{3a} R^{3b} and R^{3c}, when present, is independently selected from hydrogen, hydroxy, alkoxy, aryloxy, acyloxy, thiol, alkylthio, acylthio, arylthio, amino, alkylamino, acylamino, and alkyl;
   (2) R^{3d}, when present, is selected from hydrogen, alkyl and aryl;
   (3) R^{3e}, when present, is selected from hydrogen, alkyl aryl and acyl; and
   (4) R^{3f}, when present, is selected from hydrogen and alkyl;
(D) p i s 0, 1, 2, 3, 4 or 5; wherein
   (1) when p is greater than 0, each R⁴ and R^{4′} is independently selected from hydrogen, alkyl, aryl, halo, hydroxy, alkoxy, amino and acylamino;
   (2) when p is greater than 1, two R⁴ moieties, together with the carbon atoms to which they are bonded, can join to form a heterocycloalkyl, cycloalkyl or aryl ring; and
   (3) when p is greater than 1, the R⁴ moieties on two adjacent carbon atoms can both be nil such that a double bond is formed between the two adjacent carbon atoms, or both the R⁴ and R^{4′} moieties on two adjacent carbon atoms can all be nil such that a triple bond is formed between the two adjacent carbon atoms;
(E) R⁵ represents the 5 substituents (i.e., positions 2-6) on phenyl ring J, wherein each R⁵ is independently selected from hydrogen, hydroxy, halo, thiol, —OR¹², —SR¹², —SO₂N(R¹²)(R^{12′}), —N(R¹²)(R^{12′}), alkyl, acyl, alkene, alkyne, cyano, nitro, aryl, heteroaryl, cycloalkyl, and heterocycloalkyl; where each R¹² and R^{12′} is independently selected from hydrogen, alkyl, acyl, heteroalkyl, aryl, heteroaryl, cycloalkyl, and heterocycloalkyl; or two R⁵ moieties can optionally join to form a carbocyclic or a heterocyclic ring that is fused to phenyl ring J;
(F) q is 0, 1, 2, 3, 4 or 5; wherein
   (1) when q is greater than 0, each R⁶ and R^{6′} is independently selected from hydrogen, alkyl, aryl, halo, hydroxy, alkoxy, amino and acylamino;
   (2) when q is greater than 1, two R⁶ moieties, together with the carbon atoms to which they are bonded, can join to form a heterocycloalkyl, cycloalkyl or aryl ring; and
   (3) when q is greater than 1, the R⁶ moieties on two adjacent carbon atoms can be nil such that a double bond is formed between the two adjacent carbon atoms, or both the R⁶ and R^{6′} moieties on two adjacent carbon atoms can all be nil such that a triple bond is formed between the two adjacent carbon atoms;
(G) Ar is an aryl or heteroaryl ring selected from the group consisting of phenyl, thiophene, furan, oxazole, thiazole, pyrrole and pyridine;
(H) R⁷ represents all the substituents on ring Ar, wherein each R⁷ is independently selected from hydrogen, halo, —NR¹³R^{13′}, alkyl, acyl, alkene, alkyne, cyano, nitro, aryl, heteroaryl, cycloalkyl, and heterocycloalkyl; where each R¹³ and R^{13′} is independently selected from hydrogen, alkyl, acyl, heteroalkyl, aryl, heteroaryl, cycloalkyl, and heterocycloalkyl; or two R⁷ moieties can optionally join to form a carbocyclic or a heterocyclic ring fused to ring Ar;
(I) r is 0, 1, 2, 3, 4, 5, 6 or 7; wherein
   (1) each R⁸ and R^{8′} is independently selected from hydrogen, alkyl, halo, hydroxy, alkoxy and amino;
   (2) when r is greater than 1, two R⁸ moieties, together with the carbon atoms to which they are bonded, can join to form a heterocycloalkyl, cycloalkyl or aryl ring; and
   (3) when r is greater than 1, the R⁸ moieties on two adjacent carbon atoms can be nil such that a double bond is formed between the two adjacent carbon atoms, or both the R⁸ and R^{8′} moieties on two adjacent carbon atoms can all be nil such that a triple bond is formed between the two adjacent carbon atoms;
(J) B is selected from —N(R¹⁴)C(═NR¹⁵), ═O, or ═S)NR¹⁶R¹⁷, —NR²⁰R²¹, cyano (—CN), a heteroaryl ring eg. thiophene, an alkyl or dialkyl amine, a heteroaryl ring containing at least one ring nitrogen atom and a heterocycloalkyl ring containing at least one ring nitrogen atom, wherein R¹⁴, R¹⁵ R¹⁶, R¹⁷, R²⁰ and R²¹ are independently selected from hydrogen, alkyl, alkene, and alkyne; wherein further a combination of two or more of R¹⁴, R¹⁵, R¹⁶ and R¹⁷ may optionally combine with the atoms to which they are bonded to form a monocyclic or bicyclic ring; preferred are —N(R¹⁴)C(═NR¹⁵)NR¹⁶R¹⁷, cyano, N(R¹⁴)C(═O)NR¹⁶R¹⁷, a heteroaryl ring containing at least one ring nitrogen atom and a heterocycloalkyl ring containing at least one ring nitrogen atom. More preferred are —N(R¹⁴)C(═NR¹⁵)NR¹⁶R¹⁷, N(R¹⁴)C(═O)NR¹⁶R¹⁷, cyano, and triazole and imidazole.
(K) s is 0, 1, 2, 3, 4 or 5; wherein
   (1) when s is greater than 0, each R⁹ and R^{9′} is independently selected from hydrogen, alkyl, aryl, halo, hydroxy, alkoxy, amino and acylamino;
   (2) when s is greater than 1, two R⁹ moieties, together with the carbon atoms to which they are bonded, can join to form a heterocycloalkyl, cycloalkyl or aryl ring; and
   (3) when s is greater than 1, the R⁹ moieties on two adjacent carbon atoms can be nil such that a double bond is formed between the two adjacent carbon atoms, or both the R⁹ and R^{9′} moieties on two adjacent carbon atoms can all be nil such that a triple bond is formed between the two adjacent carbon atoms;
(L) R¹⁰ is selected from the group consisting of an optionally substituted bicyclic aryl ring and an optionally substituted bicyclic heteroaryl ring; and
(M) D is independently selected from hydrogen, fluoro, hydroxy, thiol, acylthio, alkoxy, aryloxy, alkylthio, acyloxy, cyano, amino, acylamino, —C(O)R¹¹ and —C(S)R¹¹; wherein R¹¹ is selected from the group consisting of hydroxy; alkoxy; amino; alkylamino; —NHOR¹⁸, where R¹⁸ is selected from hydrogen and alkyl; —N(R¹⁹)CH₂C(O)NH₂, where R¹⁹ is alkyl; —NHCH₂CH₂OH; —N(CH₃)CH₂CH₂OH; and —NHNHC(═Y)NH₂, where Y is selected from O, S and NH; and
(N) wherein if at least one of Z¹, Z² or Z³ is other than —C(O)N(R^{3d})— or —N(R^{3d})C(O)—, then X and D may optionally be linked together via a linking moiety, L, that contains all covalent bonds or covalent bonds and an ionic bond so as to form a cyclic peptide analog;
   or an optical isomer, diastereomer or enantiomer thereof; a pharmaceutically-acceptable salt, hydrate, or biohydrolyzable ester, amide or imide thereof.

The invention also relates to pharmaceutical compositions comprising the above compounds, and to methods of treating disorders mediated by the MC-3 or MC-4 receptor by administering these compounds.

### DETAILED DESCRIPTION OF THE INVENTION

### I. Definitions

“Amino acid” refers to alanine (Ala; A), arginine (Arg; R), asparagine (Asn; N), aspartic acid (Asp; D), cysteine (Cys; C), glutamic acid (Gln; Q), glutamine (Gin; E), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V). The common 3-letter and 1-letter abbreviations are indicated parenthetically. Modified amino acids also useful herein are the following (the 3-letter abbreviation for each moiety is noted parenthetically): p-Benzoyl-phenylalanine (Bpa); β-(1-Naphthyl)-alanine (1-Nal); β-(2-Naphthyl)-alanine (2-Nal); β-Cyclohexylalanine (Cha), 3,4-Dichlorophenylalanine (3,4-Dcp); 4-Fluorophenylalanine (4-Fpa); 4-Nitrophenylalanine (4-Npa); 2-Thienylalanine (Tha); 1,2,3,4-Tetrahydroisoquinoline-3-carboxylic acid (Tic); 3-Benzothienylalanine (3-Bal); 4-Cyanophenylalanine (4-Ypa); 4-Iodophenylalanine (4-Ipa); 4-Bromophenylalanine (4-Rpa); 4,4′-Biphenylalanine (Bip); Ornithine (Orn); Sarcosine (Sar); Pentafluorophenylalanine (Pfp); and β,β-Diphenylalanine (Dip). With respect to moieties depicted on Formula (I) and Formula (A), moieties referred to using a single letter designation are as defined and do not refer to the single letter amino acids corresponding to those letters.

The letter “D” preceding the above three-letter abbreviations, e.g. as in “D-Nal” or “D-Phe”, denotes the D-form of the amino acid. The letter “L” preceding an amino acid three-letter abbreviation denotes the natural L-form of the amino acid. For purposes of this disclosure, unless otherwise indicated, absence of a “D” or “L” designation indicates that the abbreviation refers to both the D- and L-forms. Where the common single-letter abbreviation is used, capitalization refers to the L-form and small letter designation refers to the D-form, unless otherwise indicated.

“Ac” refers to acetyl (i.e., CH₃C(═O)—).

“Acylamino” refers to R—C(═O)N—.

“Acyloxy” refers to R—C(═O)O—.

“Acylthio” refers to R—C(═O)S—.

“Alkoxy” is an oxygen radical having a hydrocarbon chain substituent, where the hydrocarbon chain is an alkyl or alkene (i.e., —O-alkyl or —O-alkene). Preferred alkoxy groups include (for example) methoxy (MeO), ethoxy, propoxy and allyloxy.

“Alkyl” is a saturated hydrocarbon chain having 1 to 15 carbon atoms, preferably 1 to 10, more preferably 1 to 4 carbon atoms. “Alkene” is a hydrocarbon chain having at least one (preferably only one) carbon-carbon double bond and having 2 to 15 carbon atoms, preferably 2 to 10, more preferably 2 to 4 carbon atoms. “Alkyne” is a hydrocarbon chain having at least one (preferably only one) carbon-carbon triple bond and having 2 to 15 carbon atoms, preferably 2 to 10, more preferably 2 to 4 carbon atoms. Alkyl, alkene and alkyne chains (referred to collectively as “hydrocarbon chains”) may be straight or branched and may be unsubstituted or substituted. Preferred branched alkyl, alkene and alkyne chains have one or two branches, preferably one branch. Preferred chains are alkyl. Alkyl, alkene and alkyne hydrocarbon chains each may be unsubstituted or substituted with from 1 to 4 substituents; when substituted, preferred chains are mono-, di-, or tri-substituted. Alkyl, alkene and alkyne hydrocarbon chains each may be substituted with halo, hydroxy, aryloxy (e.g., phenoxy), heteroaryloxy, acyloxy (e.g., acetoxy), carboxy, aryl (e.g., phenyl), heteroaryl, cycloalkyl, heterocycloalkyl, spirocycle, amino, amido, acylamino, keto, thioketo, cyano, or any combination thereof. Preferred hydrocarbon groups include methyl (Me), ethyl, propyl, isopropyl, butyl, vinyl, allyl and butenyl.

Also, as referred to herein, a “lower” alkyl, alkene or alkyne moiety (e.g., “lower alkyl”) is a chain comprised of 1 to 6, preferably from 1 to 4, carbon atoms in the case of alkyl and 2 to 6, preferably 2 to 4, carbon atoms in the case of alkene and alkyne.

“Alkylthio” is a sulfur radical having a hydrocarbon chain substituent, where the hydrocarbon chain is an alkyl or alkene (i.e., —S-alkyl or —S-alkene). Preferred alkylthio groups include (for example) methylthio (MeS) and ethylthio.

“Aryl” is an aromatic hydrocarbon ring. Aryl rings are monocyclic or fused bicyclic ring systems. Monocyclic aryl rings contain 6 carbon atoms in the ring. Monocyclic aryl rings are also referred to as phenyl rings. Bicyclic aryl rings contain from about 8 to about 17 carbon atoms, preferably about 9 to about 12 carbon atoms in the ring. Bicyclic aryl rings include ring systems wherein one ring is aryl and the other ring is aryl, cycloalkyl, or heterocycloalkyl. Preferred bicyclic aryl rings comprise 5-, 6- or 7-membered rings fused to 5-, 6-, or 7-membered rings. Aryl rings may be unsubstituted or substituted with from 1 to 4 substituents on the ring. Aryl may be substituted with halo, cyano, nitro, hydroxy, carboxy, amino, acylamino, alkyl, heteroalkyl, haloalkyl, phenyl, aryloxy, heteroaryloxy, or any combination thereof. Preferred aryl rings include naphthyl, tolyl, xylyl, and phenyl. The most preferred aryl ring radical is phenyl.

“Aryloxy” is an oxygen radical having an aryl substituent (i.e., —O-aryl). Preferred aryloxy groups include (for example) phenoxy, naphthyloxy, methoxyphenoxy, and methylenedioxyphenoxy.

As used herein, “basic amino acids” refers to His, Lys, and Arg.

“Bc” refers to butanoyl (i.e., CH₃CH₂CH₂C(═O)—).

“Cycloalkyl” is a saturated or unsaturated hydrocarbon ring. Cycloalkyl rings are not aromatic. Cycloalkyl rings are monocyclic, or are fused, spiro, or bridged bicyclic ring systems. Monocyclic cycloalkyl rings contain from about 3 to about 9 carbon atoms, preferably from 3 to 7 carbon atoms in the ring. Bicyclic cycloalkyl rings contain from 7 to 17 carbon atoms, preferably from about 7 to about 12 carbon atoms in the ring. Preferred bicyclic cycloalkyl rings comprise 4-, 5-, 6- or 7-membered rings fused to 5-, 6-, or 7-membered rings. Cycloalkyl rings may be unsubstituted or substituted with from 1 to 4 substituents on the ring. Cycloalkyl may be substituted with halo, cyano, alkyl, heteroalkyl, haloalkyl, phenyl, keto, hydroxy, carboxy, amino, acylamino, aryloxy, heteroaryloxy, or any combination thereof. Preferred cycloalkyl rings include cyclopropyl, cyclopentyl, and cyclohexyl.

“Fused” refers to cyclic moieties having at least two common ring atoms, the preferred maximum number of fused cycles being three.

“Halo” is fluoro (F), chloro (Cl), bromo (Br) or iodo (I).

“Heteroatom” is a nitrogen, sulfur, or oxygen atom, to which one or more moieties may be connected according to heteroatom valence; in the case of nitrogen, one oxygen atom may be optionally connected to it by a coordinate covalent bond, such as forming an N-oxide. Groups containing more than one heteroatom may contain different heteroatoms.

“Heteroalkyl” is a saturated or unsaturated chain containing carbon and at least one heteroatom, wherein no two heteroatoms are adjacent. Heteroalkyl chains contain from 2 to about 15 member atoms (carbon and heteroatoms) in the chain, preferably 2 to about 10, more preferably 2 to about 5. For example, alkoxy (i.e., —O-alkyl or —O-heteroalkyl) radicals are included in heteroalkyl. Heteroalkyl chains may be straight or branched. Preferred branched heteroalkyl have one or two branches, preferably one branch. Preferred heteroalkyl are saturated. Unsaturated heteroalkyl have one or more double bonds (also referred to herein as “heteroalkenyl”) and/or one or more triple bonds (also referred to herein as “heteroalkynyl”). Preferred unsaturated heteroalkyl have one or two double bonds or one triple bond, more preferably one double bond. Heteroalkyl chains may be unsubstituted or substituted with from 1 to 4 substituents. Preferred substituted heteroalkyl are mono-, di-, or tri-substituted. Heteroalkyl may be substituted with lower alkyl, halo, hydroxy, aryloxy, heteroaryloxy, acyloxy, carboxy, monocyclic aryl, heteroaryl, cycloalkyl, heterocycloalkyl, spirocycle, amino, acylamino, amido, keto, thioketo, cyano, or any combination thereof.

“Heterocycloalkyl” is a saturated or unsaturated, non-aromatic ring containing carbon and from 1 to about 4 (preferably 1 to 3) heteroatoms in the ring, wherein no two heteroatoms are adjacent in the ring and no carbon in the ring that has a heteroatom attached to it also has a hydroxyl, amino, or thiol radical attached to it. Heterocycloalkyl rings are monocyclic, or are fused, bridged, or spiro bicyclic ring systems. Monocyclic heterocycloalkyl rings contain from about 4 to about 9 member atoms (carbon and heteroatoms), preferably from 5 to 7 member atoms in the ring. Bicyclic heterocycloalkyl rings contain from about 7 to about 17 atoms, preferably from 7 to 12 atoms. Bicyclic heterocycloalkyl rings may be fused, spiro, or bridged ring systems. Preferred bicyclic heterocycloalkyl rings comprise 5-, 6- or 7-membered rings fused to 5-, 6-, or 7-membered rings. Heterocycloalkyl rings may be unsubstituted or substituted with from 1 to 4 substituents on the ring. Heterocycloalkyl may be substituted with halo, cyano, hydroxy, carboxy, keto, thioketo, amino, acylamino, acyl, amido, alkyl, heteroalkyl, haloalkyl, phenyl, phenoxy or any combination thereof. Preferred substituents on heterocycloalkyl include fluoro and alkyl.

“Heteroaryl” is an aromatic ring containing carbon and from 1 to about 4 heteroatoms in the ring. Heteroaryl rings are monocyclic or fused bicyclic ring systems. Monocyclic heteroaryl rings contain from about 5 to about 9 member atoms (carbon and heteroatoms), preferably 5 or 6 member atoms in the ring. Bicyclic heteroaryl rings contain from about 8 to about 17 member atoms, preferably about 8 to about 12 member atoms in the ring. Bicyclic heteroaryl rings include ring systems wherein one ring is heteroaryl and the other ring is aryl, heteroaryl, cycloalkyl, or heterocycloalkyl. Preferred bicyclic heteroaryl ring systems comprise 5-, 6- or 7-membered rings fused to 5-, 6-, or 7-membered rings. Heteroaryl rings may be unsubstituted or substituted with from 1 to 4 substituents on the ring. Heteroaryl may be substituted with halo, cyano, nitro, hydroxy, carboxy, amino, acylamino, alkyl, heteroalkyl, haloalkyl, phenyl, aryloxy, heteroaryloxy, or any combination thereof. Preferred heteroaryl rings include thienyl, thiazolo, imidazyl, purinyl, pyrimidyl, pyridyl, and furanyl.

As used herein, “MC-4 agonist” and “MC-3 agonist” refers to a compound with affinity for the MC-4 receptor or MC-3 receptor, respectively, that results in measurable biological activity in cells, tissues, or organisms which contain the MC-4 or MC-3 receptor. As used herein, an “MC-4/MC-3 agonist” refers to a compound that is both an MC-4 agonist and an MC-3 agonist, as those terms are defined herein. Assays which demonstrate MC-4 and/or MC-3 agonistic activity of compounds are well known in the art. One commonly used assay is the BioTrak™ cAMP direct enzymeimmunoassay (EIA) system from Amersham Pharmacia Biotech (Piscataway, N.J.), which quantitates the cAMP response of cells to MC ligands. Another useful assay is the Tropix cAMP Screen™, which is available from Tropix. These systems allow the simple quantitation of total cellular cAMP measurement in cells exposed to selective ligands. Briefly summarized: HEK cells stably transfected with the MC-1, MC-3 or MC-4 receptors are plated into 96 well microtiter plates and grown overnight. Cells are dosed with the appropriate MC ligand for 1 hour and then lysed. A fraction of the lysed cell extract is transferred to the assay plate. The ELISA assay is performed according to kit instructions. Each plate contains a series of cAMP standards for calculating a standard curve, as well as a full MC agonist as a positive control for each MC receptor. cAMP activity is calculated as a % of the maximum cAMP activity of the full MC agonist control.

As used herein, “MC-4 antagonist” and “MC-3 antagonist” refer to compounds with affinity for the MC-4 receptor or MC-3 receptor, respectively, and blocks stimulation by a known MC agonist. As used herein, an “MC-4/MC-3 antagonist” refers to a compound that is both an MC-4 antagonist and an MC-3 antagonist, as those terms are defined herein. Assays that demonstrate compounds with MC-4 and/or MC-3 antagonism are well known in the art. One particularly useful assay is competitive binding with the use of Europium-labeled NDP-MSH. Briefly summarized: HEK cells stably transfected with the MC-1, MC-3, MC-4 or MC-5 receptors are plated into 96-well microtiter plates and grown overnight. Cells are dosed with the appropriate MC ligand in the presence of europilated-NDP-MSH for 60 min., cells are washed several times, enhancement solution is added and fluorescence is measured. IC₅₀ and Ki values can be calculated at each receptor for each MC ligand using standard graphing programs such as GraphPad Prism™, (GraphPad Software Inc., San Diego, Calif.).

As used herein, “MC-3 receptor” and “MC-4 receptor” mean the known MC-3 and MC-4 receptors, their splice variants, and undescribed receptors. MC-3 receptors are described by Gantz et al., supra (human MC-3); Desarnaud et al., supra (mouse MC-3) and L. Reyfuss et al., “Identification of a Receptor for Gamma Melanotropin and Other Proopiomelanocortin Peptides in the Hypothalamus and Limbic System., Proc. Natl. Acab. Sci. USA, vol. 90, pp. 8856-8860 (1993) (rat MC-3). MC-4 receptors are described by Gantz et al., supra (human MC-4), J. D. Alvaro et al., “Morphine Down-Regulates Melanocortin-4 Receptor Expression in Brain Regions that Mediate Opiate Addiction”, Mol-Pharmacol. Sep, vol. 50(3), pp. 583-91 (1996) (rat MC-4) and Takeuchi, S. and Takahashi, S., “Melanocortin Receptor Genes in the Chicken—Tissue Distributions”, Gen-Comp-Endocrinol., vol. 112(2), pp 220-31 (November 1998) (chicken MC-4).

As used herein, “measurable” means the biologic effect is both reproducible and significantly different from the baseline variability of the assay.

A “pharmaceutically-acceptable salt” is a cationic counterion formed at any acidic (carboxylic acid) group, or an anionic counterion formed at any basic (e.g., amino) group. Many such salts are known in the art, as described in World Patent Publication 87/05297, Johnston et al., published Sep. 11, 1987 incorporated by reference herein. Preferred cationic salts include the alkali metal salts (such as sodium and potassium), and alkaline earth metal salts (such as magnesium and calcium) and organic salts. Preferred anionic salts include the halides (such as chloride salts), sulfonates, carboxylates, phosphates, trifluoroacetate (TFA) and the like. Clearly contemplated in such salts are addition salts that may provide an optical center where once there is none. For example, a chiral tartrate salt may be prepared from the compounds of the invention, and this definition includes such chiral salts.

Such salts are well understood by the skilled artisan, and the skilled artisan is able to prepare any number of salts given the knowledge in the art. Furthermore, it is recognized that the skilled artisan may prefer one salt over another for reasons of solubility, stability, formulation ease and the like. Determination and optimization of such salts is within the purview of the skilled artisan's practice.

As used herein, “selective” means having an activation preference for a specific receptor over other receptors which can be quantified based upon whole cell, tissue, or organism assays which demonstrate receptor activity, such as the cAMP enzyme immunoassay (EIA) system discussed above. A compound's selectivity is determined from a comparison of its EC₅₀ values at the relevant receptors being referenced. As used herein, unless indicated, use of the term “selective over the other MC receptors” means selective with respect to the other melanocortin receptors, including the MC-1, MC-2 and MC-5 receptors. For example, a compound having an EC₅₀ of 8 nM at the MC-4 receptor and an EC₅₀ of ≧80 nM at the MC-1, MC-2 and MC-5 receptors has a selectivity ratio for the MC-4 receptor over the other MC receptors of at least 1:10. Additionally, it will be recognized that selectivity may also refer to one of the MC-1, MC-2 or MC-5 receptors individually. For example, a compound having an EC₅₀ of 8 nM at the MC-4 receptor and an EC₅₀ of 80 nM at the MC-1 receptor has a selectivity ratio for the MC-4 receptor over the MC-1 receptor of 1:10. Such a compound is selective over the MC-1 receptor, regardless of its EC₅₀ value for MC-2 or MC-5. Selectivity is described in more detail below and may be determined by using, for example, the software Prism v 2.0 which is available from GraphPad, Inc.

A “solvate” is a complex formed by the combination of a solute (e.g., a MC-4/MC-3 receptor ligand of the present invention) and a solvent (e.g., water). See J. Honig et al., The Van Nostrand Chemist's Dictionary, p. 650 (1953). Pharmaceutically-acceptable solvents used according to this invention include those that do not interfere with the biological activity of the compound (e.g., water, ethanol, ethers, acetic acid, N,N-dimethylformamide and others known or is readily determined by the skilled artisan).

“Spirocycle” is an alkyl or heteroalkyl diradical substituent of an alkyl or heteroalkyl, wherein said diradical substituent is attached geminally and wherein said diradical substituent forms a ring, said ring containing about 4 to about 8 member atoms (carbon or heteroatoms), preferably 5 or 6 member atoms.

### II. The Compounds

The compounds of the present invention are MC-4 and/or MC-3 receptor ligands having a structure according to Formula (I): 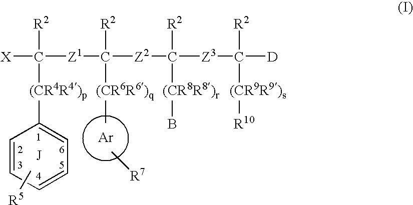
wherein R², R⁴, R^{4′}, R⁵, R⁶, R^{6′}, R⁷, R⁸, R^{8′}, R⁹, R^{9′}, R¹⁰, A, Z¹, Z², Z³, X, B, D, p, q, r and s are as described in the Disclosure of the Invention section above.

In addition to the compounds described by Formula (I), it is envisioned that the core peptidic residues can be pegylated to provide enhanced therapeutic benefits such as, for example, increased efficacy by extending half-life in vivo. Peptide pegylation methods are well known in the literature. For example, pegylation of peptides is described in the following references, the disclosure of each of which is incorporated herein by reference: Lu, Y. A. et al., “Pegylated peptides. II. Solid-phase synthesis of amino-, carboxy- and side-chain pegylated peptides”, Int. J. Pept. Protein Res., Vol. 43(2), pp. 127-38 (1994); Lu, Y. A. et al., “Pegylated peptides. I. Solid-phase synthesis of N alpha-pegylated peptides using Fmoc strategy”, Pept. Res., Vol. 6(3), pp. 140-6 (1993); Felix, A. M. et al., “Pegylated peptides. IV. Enhanced biological activity of site-directed pegylated GRF analogs.”, Int. J. Pept. Protein Res., Vol. 46(3-4), pp. 253-64 (1995); Gaertner, H. F. et al., “Site-specific attachment of functionalized poly(ethylene glycol) to the amino terminus of proteins”, Bioconjug Chem., Vol. 7(1), pp. 38-44 (1996); Tsutsumi, Y. et al., “PEGylation of interleukin-6 effectively increases its thrombopoietic potency”, Thromb Haemost, Vol. 77(1), pp. 168-73 (1997); Francis, G. E. et al., “PEGylation of cytokines and other therapeutic proteins and peptides: the importance of biological optimisation of coupling techniques”, Int. J. Hematol., Vol. 68(1), pp. 1-18 (1998); Roberts, M. J. et al., “Attachment of degradable poly(ethylene glycol) to proteins has the potential to increase therapeutic efficacy”, J. Pharm. Sci., Vol 87(11), pp. 1440-45 (1998); and Tan, Y. et al., “Polyethylene glycol conjugation of recombinant methioninase for cancer therapy”, Protein Expr. Purif, Vol. 12(1), pp. 45-52 (1998). The compounds of Formula (I) can be pegylated directly, or a “linker arm” may be added to the compounds to facilitate pegylation.

With reference to Formula (1), the following is a non-limiting list of preferred substituents:

X is selected from hydrogen, fluoro, aryloxy, acyloxy, OR¹, SR¹, —NR¹R^{1′} and —CHR¹R^{1′}. Preferred are hydrogen (when D is not also hydrogen), —NR¹R^{1′} and —CHR¹R^{1′}. More preferred are —NR¹R^{1′} and —CHR¹R^{1′}. Still more preferred is —NR¹R^{1′}.

R¹ and R^{1′} are independently selected from the group consisting of hydrogen, alkyl and acyl. Preferred is where R¹ is hydrogen or alkyl and R^{1′} is acyl.

R² is independently selected from the group consisting of hydrogen, alkyl halo and heteroalkyl. Preferred is hydrogen. Alternatively, two consecutive R² moieties, or consecutive R² and R³ moieties, may join to form a 3 to 8 membered carbocyclic or heterocyclic ring. In another alternative, the R² bonded to the carbon atom that is bonded to X and Z¹ and an R⁵ moiety can optionally join to form a carbocyclic or heterocyclic ring that is fused to phenyl ring J. In another alternative, the R² bonded to the carbon atom that is bonded to ring Ar can join with an R⁷ to form a ring fused to ring Ar. In another alternative, the R² bonded to the carbon atom that is bonded to Z² and Z³ can optionally join with R⁸ to form a carbocyclic or heterocyclic ring. In still another alternative, the R² bonded to the carbon atom that is bonded to Z³ and D can optionally join with R¹⁰ to form a carbocyclic or heterocyclic ring. With respect to the foregoing, preferred is where R² does not form a ring with another R², and where R² does not form a ring with R³, R⁷ or R⁸. More preferred is where R² also does not form a ring with R¹⁰. Preferably rings formed between R² and another moiety will have 5 to 8 ring atoms.

Each of Z¹, Z² and Z³ is independently selected from —OC(R³)(R^{3a})—; —C(R³)(R^{3a})O—; —S(O)_{d}C(R³)(R^{3a})—, where a is 0, 1 or 2; —C(R³)(R^{3a})S(O)ₐ—, where b is 0, 1 or 2; —N(R^{3e})C(R³)R^{3a})—; —C(R³)(R^{3a})N(R^{3e}); —C(O)N(R^{3d})—; —N(R^{3d})C(O)—; —C(O)C(R³)(R^{3a})—; —C(R³)(R^{3a})C(O)—; C(R³)(R^{3a})C(R^{3b})(R^{3c})—; —C(R³)═C(R^{3a})—; —C≡C—; —SO₂N(R^{3d})—; —N(R^{3d})SO₂—; —C(R³)(R^{3a})P(═O)(OR^{3f})—; —P(═O)(OR^{3f})C(R³)(R^{3a})—; —N(R^{3d})P(═O)(OR^{3f})—; P(═O)(OR^{3f})O—; —O—P(═O)(OR^{3f})—; a cycloalkyl having from 3 to 8 ring atoms and a heterocycloalkyl having from 4 to 8 ring atoms. Preferred are —OC(R³)(R^{3a})—; —C(R³)(R^{3a})O—; —S(O)ₐC(R³)(R^{3a})—, where a is 2; —C(R³)(R^{3a})S(O)_{b}—, where b is 2; —N(R^{3c})C(R³)(R^{3a})—; —C(R³)(R^{3a})N(R^{3a})—; C(O)N(R^{3d})—; —N(R^{3d})C(O)—; C(R³)(R^{3a})C(R^{3b})(R^{3c})—; C≡C—; —SO₂N(R^{3d})—; —N(R^{3d})SO₂—; —C(R³)(R^{3a})P(═O)(OR^{3f})—; and —P(═O)(OR^{3f})C(R³)(R^{3a})—. More preferred are —OC(R³)(R^{3a})—; —C(R³)(R^{3a})O—; —C(R³)(R^{3a})N(R^{3e})—; —C(O)N(R^{3d})—; —C(R³)(R^{3a})C(R^{3b})(R^{3c})—; —C(R³)═C(R^{3a})—; —SO₂N(R^{3d})— and —P(═O)(OR^{3f})C(R³)(R^{3a})—. Most preferred are —C(R³)(R^{3a})O—; —C(O)N(R^{3d})— and —C(R³)(R^{3a})C(R^{3b})(R^{3c})—. In another aspect, preferred compounds are those where at least one of Z¹, Z² or Z³ is other than —C(O)N(R^{3d})—. More preferred are compounds where at least two of Z¹, Z² or Z³ are other than —C(O)N(R^{3d})—. Also more preferred are compounds where (i) at least one of Z¹, Z² or Z³ is other than —C(O)N(R^{3d})— and (ii)(a) X is other than —NR¹R^{1′} where R^{1′} is acyl and/or (b) D is other than —C(O)R^{1′}.

Each of R³, R^{3a} R^{3b} and R^{3c}, when present, is independently selected from hydrogen, hydroxy, alkoxy, aryloxy, acyloxy, thiol, alkylthio, acylthio, arylthio, amino, alkylamino, acylamino, and alkyl. Preferred are hydrogen, hydroxy, alkoxy, aryloxy and alkyl. Most preferred is where each of R³, R^{3b} and R^{3c} is hydrogen.

R^{3d}, when present, is selected from hydrogen, alkyl and aryl. Preferred is where R^{3d} is selected from hydrogen and alkyl.

R^{3c}, when present, is selected from hydrogen, alkyl, aryl and acyl. Preferred is where R^{3e} is selected from hydrogen and alkyl.

R^{3f} is selected from hydrogen and alkyl. When R^{3f} is alkyl, preferred are branched alkyl, preferably isopropyl.

p is 0, 1, 2, 3, 4 or 5. Preferably p is 1 or 2, more preferably 1.

When p is greater than 0, each R⁴ and R^{4′} is independently selected from hydrogen, alkyl, aryl, halo (preferably fluoro), hydroxy, alkoxy, amino and acylamino. When p is greater than 1, two R⁴ moieties, together with the carbon atoms to which they are bonded, may join to form a heterocycloalkyl, cycloalkyl or aryl ring. When p is greater than 1, the R⁴ moieties on two adjacent carbon atoms can both be nil such that a double bond is formed between the two adjacent carbon atoms, or both the R⁴ and R^{4′} moieties on two adjacent carbon atoms can all be nil such that a triple bond is formed between the two adjacent carbon atoms. Preferably each R⁴, when present, is hydrogen and each R⁴, when present, is hydrogen or alky, Most preferably there is no unsaturation in the chain linking ring J to the X-containing carbon atom of Formula (I).

R⁵ represents the 5 substituents (i.e., positions 2-6) on phenyl ring J, wherein each R⁵ is independently selected from hydrogen, hydroxy, halo, thiol, —OR¹², —SR¹², —SO₂N(R¹²)(R^{12′}), —N(R¹²)(R^{12′}), alkyl, acyl, alkene, alkyne, cyano, nitro, aryl, heteroaryl, cycloalkyl, and heterocycloalkyl. Each R¹² and R^{12′} is independently selected from hydrogen, alkyl, acyl, heteroalkyl, aryl, heteroaryl, cycloalkyl, and heterocycloalkyl; or two R⁵ moieties can optionally join to form a carbocyclic or a heterocyclic ring that is fused to phenyl ring J.

Preferred R⁵ moieties are hydrogen, hydroxy, halo, thiol, —OR¹² where R¹² is lower alkyl or acyl, —SR¹² where R¹² is lower alkyl or acyl, —SO₂N(R¹²)(R^{12′}), —N(R¹²)(R^{12′}), alkyl, cyano, nitro, aryl, heteroaryl, cycloalkyl, and heterocycloalkyl. More preferred R⁵ moieties are hydrogen, hydroxy, halo, thiol, —SO₂N(R¹²)(R^{12′}) where R¹² and R^{12′} both are hydrogen, —N(R¹²)(R^{12′}) where R¹² and R^{12′} each are hydrogen or alkyl. Still more preferred R⁵ moieties are hydrogen, hydroxy, chloro, fluoro, —N(R¹²)(R^{12′}) where R¹² and R^{12′} each are hydrogen or alkyl. Most preferred R⁵ moieties are hydrogen, hydroxy, chloro, fluoro and nitro.

With respect to ring J, preferred is where four of the R⁵ moieties are hydrogen. Also preferred is where the 4-position is other than hydrogen. Most preferred is where the 4-position is other than hydrogen and the remaining 4 substituents are hydrogen.

q is 0, 1, 2, 3, 4 or 5. Preferably, q is 0, 1 or 2, more preferably q is 1.

When q is greater than 0, each R⁶ and R^{6′} is independently selected from hydrogen, alkyl, aryl, halo (preferably fluoro), hydroxy, alkoxy, amino and acylamino. When q is greater than 1, two R⁶ moieties, together with the carbon atoms to which they are bonded, can join to form a heterocycloalkyl, cycloalkyl or aryl ring. When q is greater than 1, the R⁶ moieties on two adjacent carbon atoms can be nil such that a double bond is formed between the two adjacent carbon atoms, or both the R⁶ and R^{6′} moieties on two adjacent carbon atoms can all be nil such that a triple bond is formed between the two adjacent carbon atoms. Preferably each R⁶, when present, is hydrogen and each R⁶, when present, is hydrogen or alkyl. Most preferably there is no unsaturation in the chain linking the ring Ar to the carbon atom of Formula (I) that is bonded to Z¹ and Z².

Ar is an aryl or heteroaryl ring selected from the group consisting of phenyl, thiophene, furan, oxazole, thiazole, pyrrole and pyridine. Ar is preferably phenyl, thiophene or furan. Ar is most preferably phenyl.

R⁷ represents the substituents on the Ar ring, wherein each R⁷ is independently selected from hydrogen; halo; —NR¹³R^{13′} where R¹³ and R^{13′} each are hydrogen or alky; alkyl; acyl; alkene; alkyne; cyano; nitro; aryl; heteroaryl; cycloalkyl and heterocycloalkyl. Optionally, two R⁷ moieties can join to form a carbocyclic or a heterocyclic ring fused to ring Ar. When Ar is phenyl, preferred is where four of the five R⁷ moieties are hydrogen or all five of the R⁷ moieties are hydrogen. Also preferred is where two R⁷ moieties are selected from fluoro, chloro, cyano, bromo, iodo, nitro, alkoxy and alkyl; or two R⁷ moieties join to form a carbocyclic or a heterocyclic ring fused to the phenyl ring. More preferred is where the 4-position of the phenyl ring is hydrogen, fluoro, chloro, cyano, bromo, iodo, nitro and alkyl and the remaining four positions are hydrogen. Most preferred is where the 4-position of the phenyl ring is hydrogen or fluoro and the remaining four substituents are hydrogen.

When Z^{t} and Z² are both —C(O)N(R^{3d})—, peferred are compounds where the carbon atom that is bonded to Z¹ and Z² is assigned an R configuration according to Cahn-Ingold-Prelog rules of nomenclature.

r is 0, 1, 2, 3, 4, 5, 6 or 7. Preferred r is 2, 3, 4 or 5. More preferred r is 2, 3 or 5. Most preferred r is 3.

Each R⁸ and R^{8′} is independently selected from hydrogen, alkyl, halo (preferably fluoro), hydroxy, alkoxy and amino. Optionally, when r is greater than 1, two R⁸ moieties, together with the carbon atoms to which they are bonded, join to form a heterocycloalkyl, cycloalkyl or aryl ring. Preferably, each R⁸ and R^{8′} is independently selected from hydrogen and alkyl. Most preferably, each R⁸ and R^{8′} is hydrogen. Optionally, when r is greater than 1, the R⁸ moieties on two adjacent carbon atoms can be nil such that a double bond is formed between the two adjacent carbon atoms, or both the R⁸ and R^{8′} moieties on two adjacent carbon atoms can all be nil such that a triple bond is formed between the two adjacent carbon atoms.

B is selected from —N(R¹⁴)C(═NR¹⁵), ′O, or ═S)NR¹⁶R¹⁷, —NR²⁰R²¹, cyano (—CN), a heteroaryl ring eg. thiophene, an alkyl or dialkyl amine, a heteroaryl ring containing at least one ring nitrogen atom and a heterocycloalkyl ring containing at least one ring nitrogen atom. Preferred are —N(R¹⁴)C(═NR¹⁵)NR¹⁶R¹⁷, a heteroaryl ring containing at least one ring nitrogen atom and a heterocycloalkyl ring containing at least one ring nitrogen atom. More preferred are —N(R¹⁴)C(═NR¹⁵)NR¹⁶R¹⁷ cyano, N(R¹⁴)C(═O)NR¹⁶R¹⁷, a heteroaryl ring containing at least one ring nitrogen atom and a heterocycloalkyl ring containing at least one ring nitrogen atom. More preferred are —N(R¹⁴)C(═NR¹⁵)NR¹⁶R¹⁷, N(R¹⁴)C(O)NR¹⁶R¹⁷, cyano, and triazole and imidazole.

R¹⁴ and R¹⁵ are independently selected from hydrogen, alkyl, alkene, and alkyne. Preferred are hydrogen and alkyl. R¹⁶ and R¹⁷ are independently selected from hydrogen, alkyl, alkene, and alkyne. Preferred are hydrogen and alkyl. R²⁰ and R²¹ are independently selected from hydrogen, alkyl, alkene, and alkyne. Preferred are hydrogen and alkyl.

Alternatively, a combination of two or more of R¹⁴, R¹⁵, R¹⁶ and R¹⁷ combine to form a monocyclic or bicyclic ring. For example, R¹⁴ and R¹⁵, together with the atoms to which they are bonded, can join to form a heterocycloalkyl or a heteroaryl. Also, R¹⁴ and R¹⁶, together with the atoms to which they are bonded, can join to form a heterocycloalkyl or a heteroaryl. Also, R¹⁵ and R¹⁶, together with the atoms to which they are bonded, can join to form a heterocycloalkyl or a heteroaryl. Also, R¹⁶ and R¹⁷ can optionally join to form a heteroaryl or heterocycloalkyl ring. Preferred is where R¹⁵ and R¹⁶ join to form a ring.

s is 0, 1, 2, 3, 4 or 5. Preferably s is 1 or 2, more preferably 1.

When s is greater than 0, each R⁹ and R^{9′} is independently selected from hydrogen, alkyl, aryl, halo (preferably fluoro), hydroxy, alkoxy, amino and acylamino. Preferably each R⁹, when present, is hydrogen and each R^{9′}, when present, is hydrogen or alkyl. Optionally, when s is greater than 1, two R⁹ moieties, together with the carbon atoms to which they are bonded, join to form a heterocycloalkyl, cycloalkyl or aryl ring. Also, when s is greater than 1, the R⁹ moieties on two adjacent carbon atoms can be nil such that a double bond is formed between the two adjacent carbon atoms. Also, when s is greater than 1 both the R⁹ and R^{9′} moieties on two adjacent carbon atoms can all be nil such that a triple bond is formed between the two adjacent carbon atoms. Most preferably there is no unsaturation in the chain linking R¹⁰ to the D-containing carbon atom of Formula (I).

R¹⁰ is selected from the group consisting of an optionally substituted bicyclic aryl ring and an optionally substituted bicyclic heteroaryl ring. Preferred bicyclic aryl rings include 1-naphthyl, 2-naphthyl, indan, 1H-indene, benzocylcobutane and benzocylcobutene. Preferred bicyclic heteroaryl rings include indole, indoline, pyrindine, dihydropyrindine, octahydropyrindine, benzothiophene, benzofuran, benzimidazole, benzopyran, quinoline, quinolone and isoquinoline. More preferred is where R¹⁰ is 1-naphthyl, 2-naphthyl, indole, indan, 1H-indene, benzothiophene, benzofuran and benzopyran. Most preferred is where R¹⁰ is 1-naphthyl, 2-naphthyl or indole (particularly 3-indole).

D is selected from hydrogen, fluoro, hydroxy, thiol, alkoxy, aryloxy, alkylthio, acyloxy, cyano, amino, acylamino, —C(O)R¹¹ and —C(S)R¹¹. Preferred are fluoro, hydroxy, thiol, alkoxy, aryloxy, alkylthio, acyloxy, cyano, amino, acylamino, —C(O)R¹¹ and —C(S)R¹¹. More preferred are alkoxy, cyano, amino, acylamino, —C(O)R¹¹ and —C(S)R¹¹. Still more preferred are —C(O)R¹¹ and —C(S)R¹¹. Most preferred is —C(O)R¹¹.

R¹¹ is selected from the group consisting of amino; alkylamino; —NHOR¹⁸, where R¹⁸ is selected from hydrogen and alkyl; —N(R¹⁹)CH₂C(O)NH₂, where R¹⁹ is alkyl (preferably lower alkyl); —NHCH₂CH₂OH; —N(CH₃)CH₂CH₂OH; and —NHNHC(═Y)NH₂, where Y is selected from O, S and NH. Preferred R¹¹ are amino; alkylamino; —NHOR¹⁸, where R¹⁸ is selected from hydrogen and alkyl (preferably hydrogen); —N(R¹⁹)CH₂C(O)NH₂, where R¹⁹ is alkyl (preferably lower alkyl); —NHCH₂CH₂OH; and —N(CH₃)CH₂CH₂OH. More preferred R¹¹ are amino; alkylamino; —NHOR¹⁸, where R¹⁸ is selected from hydrogen and alkyl (preferably hydrogen); and —N(R¹⁹)CH₂C(O)NH₂, where R¹⁹ is alkyl. Most preferred are amino and alkylamino.

As is indicated with respect to Formula (I), if at least one of Z¹, Z² or Z³ is other than —C(O)N(R^{3d})— or —N(R^{3d})C(O)—, then X and D may optionally be linked together via a linking moiety, L, that contains all covalent bonds or covalent bonds and an ionic bond so as to form a cyclic peptide analog. Such cyclic peptides have a structure according to the following Formula (II):

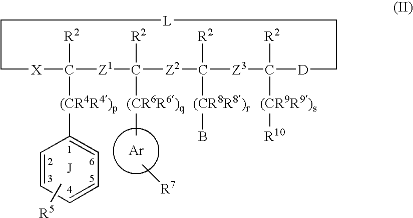

With respect to cyclic compounds containing linking moiety L, the bridge connecting X and D can be in the form of covalent bond linkages or alternatively can include a salt bridge resulting from the formation of ionic bonds. The linking moiety can be wholly peptidic in nature (i.e., containing amino acids only), non-peptidic (i.e., containing no amino acids) in nature, or it can include both peptidic and non-peptidic moieties introduced using well-known chemistry. The linking moiety can comprise aliphatic residues, aromatic residues or heteroaromatic residues, or any combination thereof. In one embodiment, the linking moiety will preferably comprise long chain omega-amino acids in which amino and carboxyl groups are separated by from about 4 to about 24 methylene groups or a combination of said omega-amino acids and aminobenzoic acids.

In another embodiment, which is a preferred embodiment, the linking moiety will contain all covalent bonds, such as amide bonds. For example, the linking moiety can comprise an amide formed through the chemical coupling of a side-chain amino group of amino acids such as Lys or Orn, and a side-chain carboxyl group of the amino acid residue such as Asp or Glu. Alternatively, the linking moiety can comprise an amide formed between the amino and carboxylate groups attached to the (t-carbon of the bridging moiety amino acids. (Hereafter referred to as the “α-amino” moiety of an amino acid or the “α-carboxyl” moiety of an amino acid.) In another alternative, the linking moiety can comprise an amide formed between any combination of the side-chain amino group or side-chain carboxyl group and the α-amino and the α-carboxyl moieties. The linking residues may be amine- or carboxyl-containing structures other than natural amino acids, including, e.g., 6-aminohexanoic acid as an amine-containing residue and succinic acid as a carboxyl-containing residue. Furthermore, the invention allows for linking using other types of chemical functionalities. In this case, these linking residues may contain a variety of groups and substituents, including aliphatic, heteroalkyl, aromatic and heterocyclic moieties. When covalently linked, the linking moiety can include but is not limited to amide, ester, ether, thioether, aminoalkyl, aminoaryl, alkyl, other heteroalkyl, alkene, alkyne, heterocycloalkyl, aryl, and heteroaryl. Preferably, the linking moiety can include ether, aminoalkyl, aminoaryl, alkyl, other heteroalkyl, alkene, alkyne, heterocycloalkyl, aryl, and heteroaryl. More preferably, the linking moiety can include ether, aminoalkyl, alkyl, alkene, and alkyne. When L contains only covalent bonds, preferred are compounds having from about 12 to about 32 ring atoms, more preferred are compounds having from about 22 to about 28 ring atoms.

The linking moiety can alternatively include an ionic bond/association that favors a cyclic structure. This “ionic” bridge is comprised of salt-forming basic and acid functionalities. For example, the link can comprise an ionic bond formed between the side-chain amino group of amino acids such as Lys or Orn, and the side-chain carboxyl group of the amino acid residue such as Asp or Glu. Alternatively, the linking moiety can comprise an ionic bond formed between the amino and carboxylate groups attached to the α-carbon of the linking moiety amino acids. In still another alternative, the linking moiety may comprise an amide formed between any combination of the side-chain amino group or side-chain carboxyl and the α-amino and the α-carboxyl moieties. When L contains an ionic bond, the ring formed will preferably contain from about 22 to about 28 ring atoms.

It will be recognized that any free peptidic α-carboxy and α-amino groups (i.e., amino acid α-carboxy and α-amino groups) not involved in formation of the ring can optionally be in the form of a carboxyamide or an acylamino moiety, respectively. The most preferred L-containing compounds are analogs wherein X and D form covalently bonded cyclic structures.

With respect to the present compounds in general, while alkyl, heteroalkyl, cycloalkyl, and heterocycloalkyl groups may be substituted with hydroxy, amino, and amido groups as stated above, the following are not envisioned in the invention:
1. Enols (OH attached to a carbon bearing a double bond).
2. Amino groups attached to a carbon bearing a double bond (except for vinylogous amides).
3. More than one hydroxy, amino, or amido attached to a single carbon (except where two nitrogen atoms are attached to a single carbon atom and all three atoms are member atoms within a heterocycloalkyl ring).
4. Hydroxy, amino, or amido attached to an sp³-hybridized carbon that also has a heteroatom attached to it.
5. Hydroxy, amino, or amido attached to a carbon that also has a halogen attached to it.

A preferred subclass of compounds of Formula (l) wherein there is no linking moiety L to form a macrocyclic ring are compounds having a structure of Formula (A) as follows:

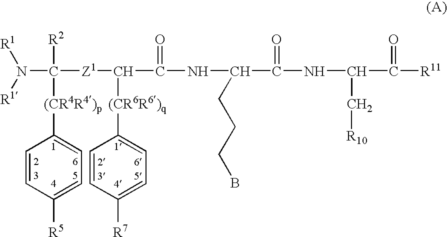

In this preferred subgenus of compounds, the moieties R¹, R^{1′}, Z¹, R⁴, R^{4′}, R⁵,R⁶, R^{6′}, R⁷, B, R¹⁰ and R¹¹ are as defined with respect to Formula (I). With reference to Formula (I), the compounds of Formula (A) are those where ring J of Formula is a phenyl ring wherein all of positions 2, 3, 5 and 6 are hydrogen, such that the ring is only substituted at the 4-position with the R⁵ moiety, which is as defined with respect to Formula (I). Similar to the description of Formula (I), the R⁵ ring moiety and the R² substituent can optionally join to form a ring fused to the depicted phenyl ring. In such an embodiment, the fused ring may join the phenyl ring at a position other than the 4-position. In Formula (A), ring Ar of Formula (I) is a phenyl ring where all of positions 2′, 3′, 5′ and 6′ are hydrogen and position 4′ is R⁷ which is as defined above. In this regard, preferred are where R⁷ is selected from hydrogen and fluoro.

With respect to Formula (A), p and q are independently 1 or 2, preferably q is 1. Also preferred are where R⁴, R^{4′}, R⁶ and R^{6′} are all hydrogen. Also preferred are compounds where B is —N(R¹⁴)C(═NR¹⁵)NR¹⁶R¹⁷ or —NR²⁰R²¹.

A preferred sub-class of compounds of Formula (II) are compounds having a structure according to Formula (B), as follows: 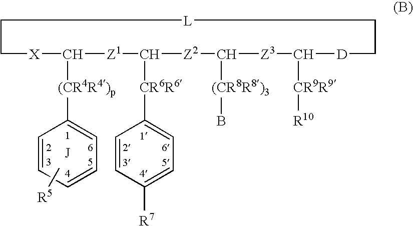
where X, Z¹, Z², Z³, D, R⁴, R^{4′}, R⁵, R⁶, R^{6′}, R⁷, R⁸, R^{8′}, B, R⁹, R^{9′}, and R¹⁰ are as defined above and p is 1 or 2. Preferred are compounds where R⁶ and R^{6′} are both hydrogen. Also preferred are compounds where B is —N(R¹⁴)C(═NR¹⁵)NR¹⁶R¹⁷ or —NR²⁰R²¹. Also preferred is where R⁸, R^{8′}, R⁹ and R^{9′} are all hydrogen. Preferably R⁷ is selected from hydrogen and fluoro. Preferred are —N(R¹⁴)C(═NR¹⁵)NR¹⁶R¹⁷, cyano, N(R¹⁴)C(═O)NR¹⁶R¹⁷, a heteroaryl ring containing at least one ring nitrogen atom and a heterocycloalkyl ring containing at least one ring nitrogen atom. More preferred are N(R¹⁴)C(═NR¹⁵)NR¹⁶R¹⁷, N(R¹⁴)C(═O)NR¹⁶R¹⁷, cyano, and triazole and imidazole.

The following is a non-limiting list of preferred compounds of Formula (I). With respect to the compounds depicted with chemical structures, included are both “linear” compounds (i.e., those where linking moiety L is not present to provide a macrocyclic molecule) and macrocyclic compounds of Formula (II).

The following list uses the one- and/or three-letter amino acid abbreviations discussed above.

| | |
|---|---|
| YfRW-NH₂ | Ac-YfRW-NH₂ |
| Y(2-Nal)RW-NH₂ | Ac-YfRW |
| Y(1-Nal)RW-NH₂ | Ac-Y(D-1-Nal)RW-NH₂ |
| Ac-a[DYfRWK]-NH₂ | Ac-a[DY(D-Phe(4-Cl))RWK]-NH₂ |
| Ac-Y(2-Nal)RW-NH₂ | Ac-[EYfRWGK]-NH₂ |
| Ac-Y(D-2-Nal)RW-NH₂ | Ac-YFRW-NH₂ |
| Ac-Y(2-Nal)RW | Ac-Y(D-Phe(4-F))RW-NH₂ |
| Ac-(Phe(4-F))fRW-NHCH₃ | Ac-(Phe(4-Cl))fRW-NH₂ |
| Ac-(Phe(4-Cl))fRW-NHCH₃ | Ac-YfKW-NH₂ |
| Ac-YfK(2-Nal)-NH₂ | Ac-YfK(2-Nal)-NHCH₃ |
| Ac-YfR(2-Nal)-NHCH₃ | Ac-YfR(2-Nal)-NH₂ |
| Ac-YfR(1-Nal)-NH₂ | (des- NH₂Tyr)YfR(2-Nal)-NHCH₃ |
| Ac-TlCfRW-NHCH₃ | Ac-a[EYfRWGK]-NH₂ |
| Ac-(Phe(4-NO₂))fRW-NH₂ | Ac--(Phe(4-Cl))(D-Phe(4-F))RW-NH₂ |
| Ac--(Phe(4-Cl))(D-Phe(4-F))RW-NHCH₃ | Bc-YfRW(Sar)-NH₂ |
| Ac-(Phe(4-Cl))fR(2-Nal)-NHCH₃ | Bc-(Phe(4-Cl))(fRW(Sar)-NH₂ |
| Ac-YfHW-NH₂ | Ac-Yf(homo-His)W-NH₂ |
| Ac-(Phe(4-Cl))(Phe(4-F)R(2-Nal)-NHCH₃ | Ac-Y(Phe(4-F)R(2-Nal)-NHCH₃ |
| Ac-FfRW-NH₂ | Ac-FfR(2-Nal)-NHCH₃ |
| Ac-yfRW-NH₂ | yfRW-NH₂ |
| Ac-YyRW-NH₂ | Ac-Y(D-Phe(4-I))KW-NH₂ |
| Ac-Y(D-Phe(4-I))HW- NH₂ | Ac-Y(D-Phe(4-I))RW-NH₂ |
| Ac-WfRW-NH₂ | Ac-YfR(Trp(5-F)-NH₂ |
| Ac-Y(D-Phe(4Br))RW-NH₂ | Ac-(D-Phe(3-OH))fRW-NH₂ |
| Ac-F(D-Phe(4-I))KW]-NH₂ | Ac-YfR(Trp(5 -OMe)-NH₂ |
| Ac-YfR(Trp(5-Br)-NH₂ | Ac-YfR(Trp(5-Me)-NH₂ |
| Ac-a[DYfR(Trp(6-F)GK]-NH₂ | Ac-YfR(Trp(1-Me)-NH₂ |
| Ac-a[DYfR(Trp(4-F)GK]-NH₂ | Ac-YfR(Trp(6-Br)-NH₂ |
| Ac-a[DYfR(Trp(7-Me)GK]-NH₂ | Ac-YfR(Trp(5-OH))-NH₂ |
| Ac-YfR(Trp(6-OH)) -NH₂ | Ac-YfR(Trp(6-Cl))-NH₂ |
| Ac-(Tyr(Me))fRWG-NH₂ | Ac-(Tyr(CH₂Ph))fRW-NH₂ |
| Ac-(Tyr(3-NH₂))fRW-NH₂ | Ac(Tyr(3-Cl))fRW-NH₂ |
| Ac-(Tyr(3-MeO))fR-NH₂ | Ac-Y(D-Phe(5-F))RW-NH₂ |

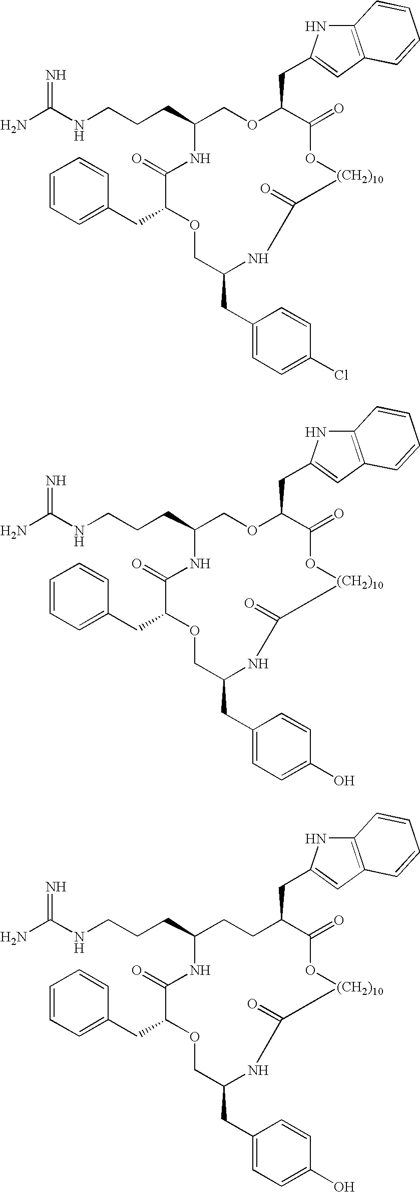

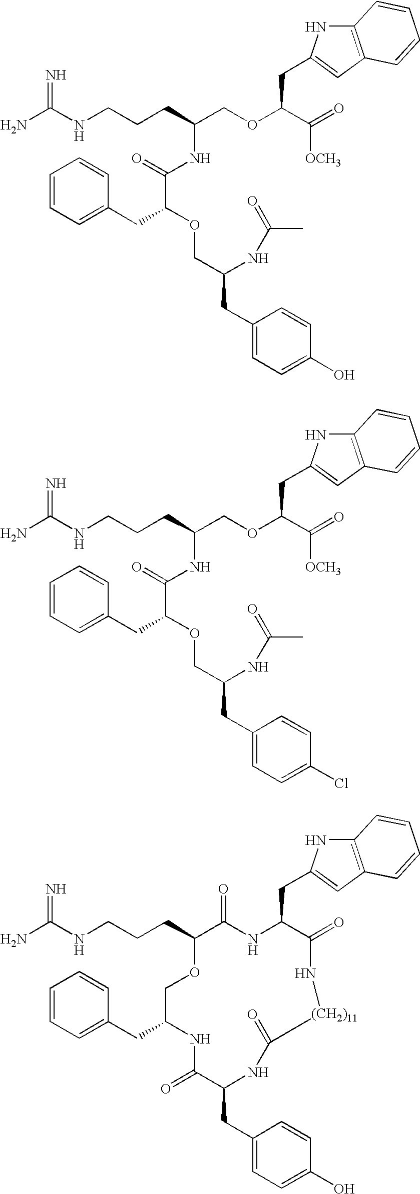

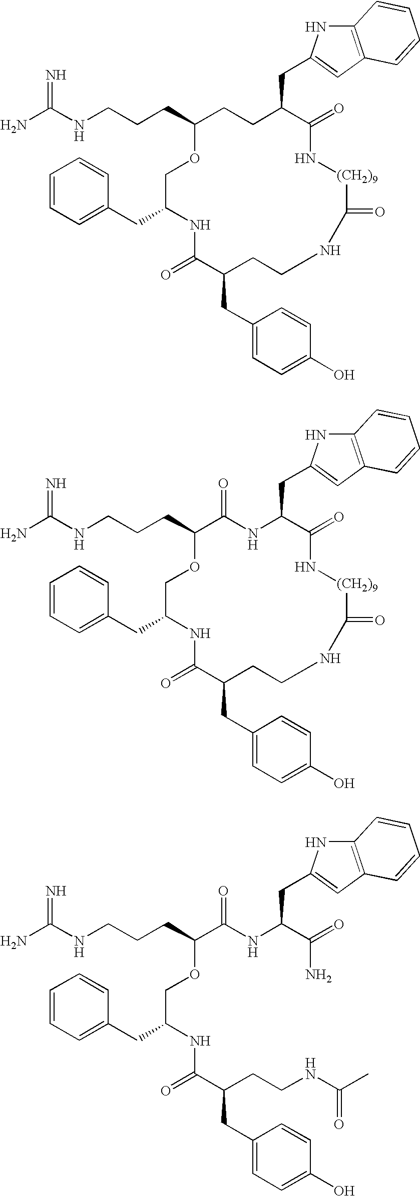

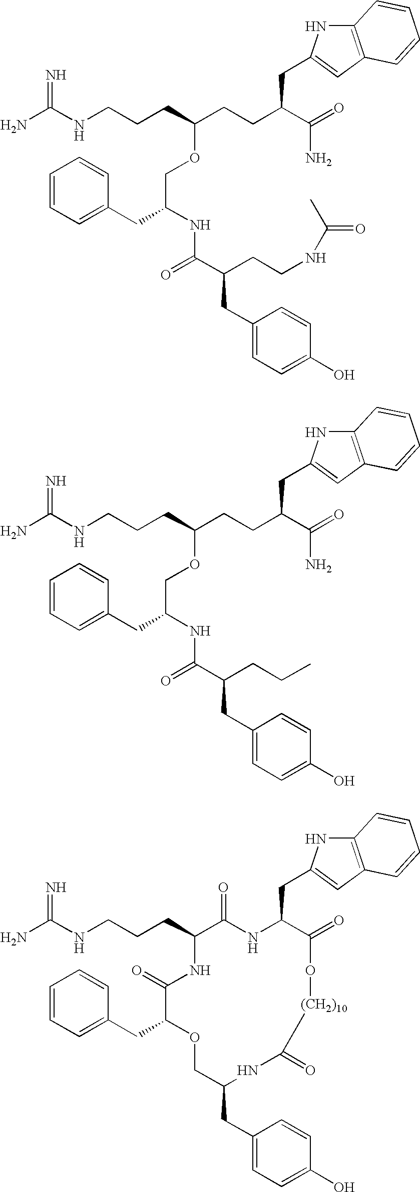

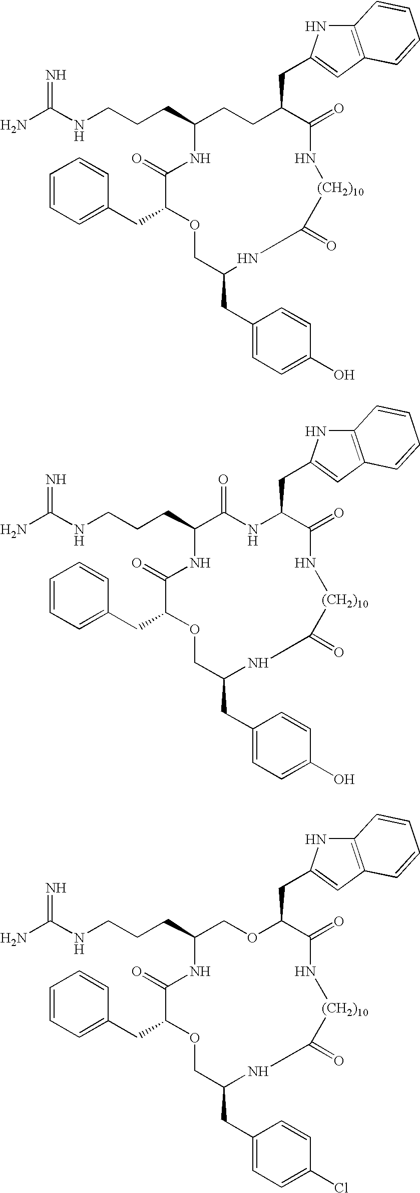

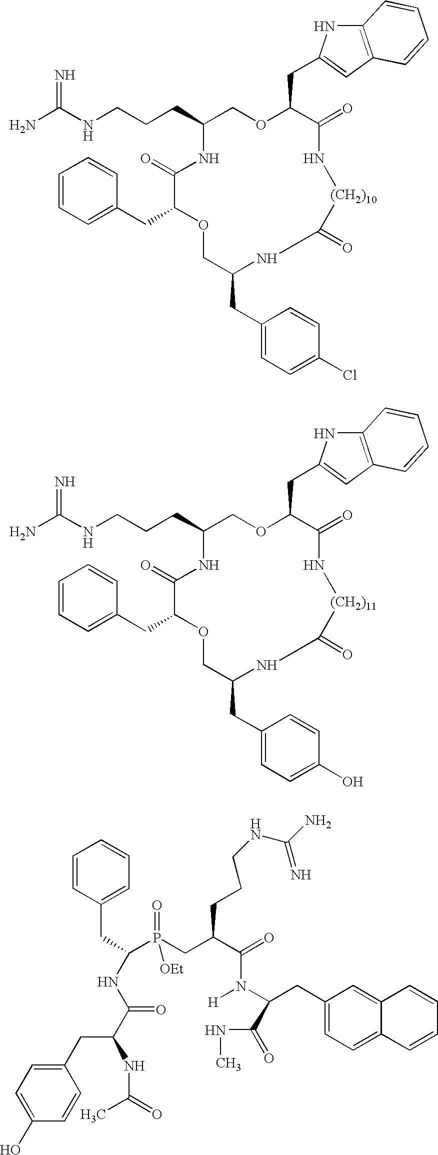

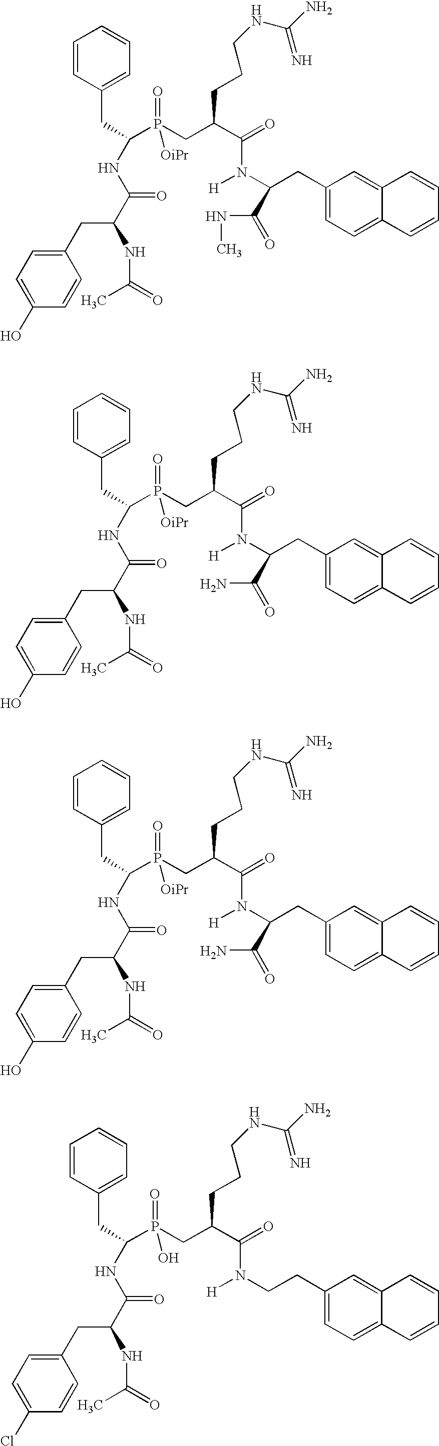

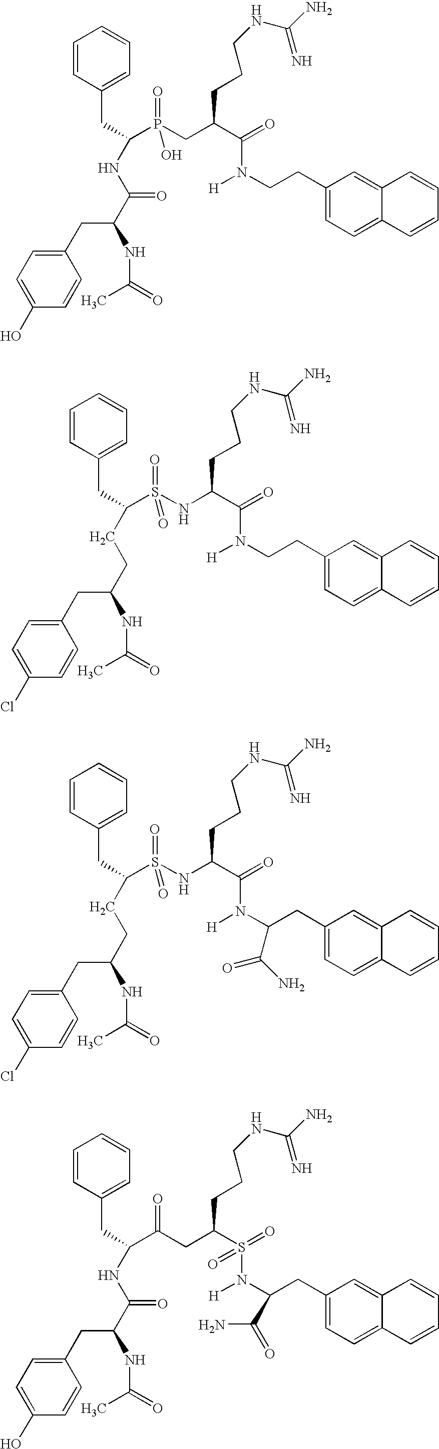

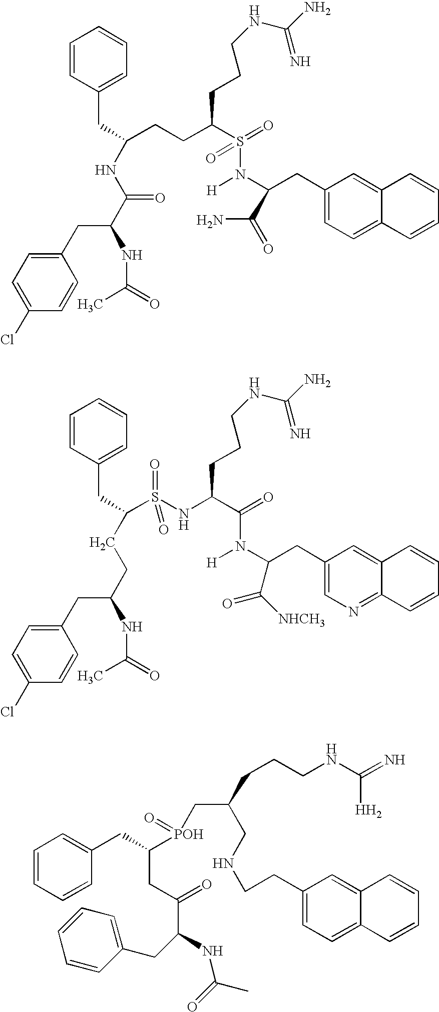

### III. Synthesis of the Compounds

The compounds of the present invention can be prepared using a variety of procedures, including solid phase and solution phase techniques. A general description of both the solid and solution phase techniques is set forth below. In Section VII, several representative examples are set forth for each of these synthetic techniques.

A. Solid Phase Chemistry

Linear Peptide Synthesis: The compounds are synthesized manually (a brief description is set forth in Section VII-C below) or automatically either with a Perkin-Elmer Applied Biosystem Division (PE-ABD) Model 433 automated synthesizer or with a SyntraPrep Reaction Station (from SyntraChem, Charlottesville, Va.). All the reagents used for peptide synthesis, can be purchased from PE-ABD. Standard 0.25 mmole FastMoc conductivity monitoring chemistry with single coupling is used with the PE-ABD automated synthesizer. The general Fmoc chemistry protocol for SPPS (solid phase peptide synthesis) includes: 1) cleavage of the Fmoc protection groups with piperidine; 2) activation of the carboxyl group of amino acids; and 3) coupling of the activated amino acids to the amino-terminal of the resin bound peptide chain to form peptide bonds. FastMoc cycles in which amino acids are activated with 2-(1H-benzotriazol-1-yl)-1,1,3,3-tetramethyluronium hexafluorophosphate (HBTU). 1.0 mmole of dry protected amino acid in a cartridge is dissolved in a solution of HBTU, N,N-diisopropylethylamine (DIEA), and 1-hydroxybenzotriazole (HOBt) in N,N-dimethylformamide (DMF) with additional N-methylpyrrolidone (NMP) added. The activated Fmoc amino acid is formed almost instantaneously and the solution is transferred directly to the reaction vessel. The step of Fmoc deprotection is monitored and controlled by conductivity measurement. The peptide chain is built on a Rink Amide resin since the C-terminal amide is needed. The acetyl or butyl group is added on the N-terminal side of the peptide after the full length of the peptide chain is made. It is accomplished by reaction of acetic anhydride or butyric anhydride (4.75% V:V acetic anhydride or butyric anhydride, 0.2% HOBt W:V, 2.25% DIEA in NMP) with the α-amino group on N-terminal side of residue. The final synthesis product is washed extensively with NMP and dichloromethane (DCM). While the SyntraChem reaction station is used for peptide synthesis, O-(7-azabenzotriazol-1-yl)-1,1,3,3-tetramethyluronium hexafluoraphosphate (HATU) is used as an activation reagent to replace HBTU.

Deprotection: The resins containing synthesized peptides are unloaded from the synthesizer and briefly air-dried. Using 4.0-10.0 ml of the cleavage cocktail (91% trifluoroacetic acid (TFA), 2.3% ethanodithiol, 2.3% thioanisol, and 2.3% phenol (W:V) in water) for 1.5-3.0 hours at room temperature, the peptides are cleaved off the resin and at the same time, the side chain protection groups [O-t-butyl (OtBu) for Asp, Glu, Tyr and Ser, pentamethyldihydrobenzofuran-5-sulfonyl (Pbf) for Arg, t-butoxycarbonyl (Boc) for Trp, Orn, Lys] are removed under the deprotection conditions. The cleavage solution is separated from the resin by filtration. The peptide in the filtrate is then precipitated by adding 40 ml cold ether. Peptide precipitate is filtered and washed with 4×40 ml of cold ether. For the peptides that do not precipitate in the ether solution due to their high degree of hydrophobicity, nitrogen stream is delivered to evaporate the ether. The peptides are then frozen and lyophilized for more than 24 hrs. The peptides are recovered into the solution by adding acetic acid.

Purification and Characterization: The peptide powder along with other by-products are re-dissolved in 50% acetic acid solution and injected onto a Vydac 1.0 cm I.D. 25 cm length C-8 column with 5 μm particle size, and 300 Å pore size for purification. A Beckman System Gold HPLC system with dual wavelength u.v. detector is used. Linear gradient of acetonitrile is programmed and introduced to the column to separate the peptide product from other substances. The elute is collected by a Pharmacia fraction collector, and the individual separation fractions are subjected to both analytical HPLC and electrospray MS for characterization to ensure the identity and purity.

B. Solution Phase Chemistry

Solvents and reagents commercially obtained are used without purification. Reaction mixtures are stirred magnetically and are monitored by either analytical high performance liquid chromatography (HPLC) or thin-layer chromatography (TLC). Solutions are routinely concentrated using a Büchi rotary evaporator at 15-25 mm Hg. TLC is performed using silica gel 60 F₂₅₄ precoated plates with a fluorescent indicator. Visualization is accomplished routinely with UV light (254 nm.) Flash column chromatography is carried out with E. Merck silica gel 60 (230-400 mesh) using the eluants indicated; chromatographic separations are monitored by TLC analyses. Analytical HPLC is carried out on either 4.6×250 mm MetaChem Kromasil C₄- or Polaris C₁₈-reverse-phase columns (3.5μ or 3.0μ particle sizes for C₄ or C₁₈, respectively) using a 0.1% phosphoric acid in water (A)/acetonitrile (B) gradient (5% B for C₄ or 20% B for C₁₈ to 100 % B over 20 min, hold 5 min) with a flow rate of 1.0 ml/min; detection is by UV light at both 214 and 254 nm. Preparative HPLC is conducted on either a 50×250 mm Polaris Cig-reverse-phase column (10μ particle size, 100 Å pore size) or a 41.4×250 mm Rainin Dynamax C₄-reverse-phase column (8μ particle size, 300 Å pore size) using a 0.1% trifluoroacetic acid in water(A)/acetonitrile(B) gradient (5% to 100% B over 55 min, hold 10 min); detection is by UV light at 214 nm.

C. General

It is recognized that it is preferable to use a protecting group for any reactive functionality such as a carboxyl, hydroxyl and the like. This is standard practice, well within the normal practice of the skilled artisan.

The indicated steps may be varied to increase yield of desired product. The skilled artisan will recognize the judicious choice of reactants, solvents, and temperatures is an important component in any successful synthesis. Determination of optimal conditions, etc. is routine. Thus the skilled artisan can make a variety of compounds using the guidance of the above general descriptions, together with the teachings of the examples in Section VII.

It is recognized that the skilled artisan in the art of organic chemistry can readily carry out standard manipulations of organic compounds without further direction; that is, it is well within the scope and practice of the skilled artisan to carry out such manipulations. These include, but are not limited to, reduction of carbonyl compounds to their corresponding alcohols, oxidations of hydroxyls and the like, acylations, aromatic substitutions, both electrophilic and nucleophilic, etherifications, esterification and saponification and the like. Examples of these manipulations are discussed in standard texts such as March, Advanced Organic Chemistry (Wiley), Carey and Sundberg, Advanced Organic Chemistry (Vol. 2) and other art that the skilled artisan is aware of.

The skilled artisan will also readily appreciate that certain reactions are best carried out when potentially reactive functionalities on the molecule are masked or protected, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene, Protecting Groups in Organic Synthesis. Of course, amino acids with reactive side chains used as starting materials are preferably blocked to prevent undesired side reactions.

### IV. Melanocortin Functional Activity and Selectivity

Functional activity can be evaluated using various methods known in the art. Examples of such methods are measurement of second messenger responses, in particular cAMP, the use of modified cell systems yielding color reaction upon accumulation of second messenger elements such as cAMP, e.g. as described by Chen et al. 1995 (Anal Biochem. 1995, 226, 349-54), Cytosensor Microphysiometer techniques (see Boyfield et al. 1996), or the study of physiological effects caused by the compounds of the invention may be applied by using the compounds of the invention alone, or in combination with natural or synthetic MSH-peptides.

The compounds of the present invention will interact preferentially (i.e., selectively) to MC-4 and/or MC-3, relative to the other melanocortin receptors. Selectivity is particularly important when the compounds are administered to humans or other animals, to minimize the number of side effects associated with their administration. MC-3/MC-4 selectivity of a compound is defined herein as the ratio of the EC₅₀ of the compound for an MC-1 receptor (“EC₅₀-MC-1”) over the EC₅₀ of the compound for the MC-3 (EC₅₀-MC-3)/MC-4 (EC₅₀-MC-4) receptor, the EC₅₀ values being measured as described above. The formulas are as follows:
MC-3 selectivity=[EC₅₀-MC-1]/[EC₅₀-MC-3]
MC-4 selectivity=[EC₅₀-MC-1]/[EC₅₀-MC-4]

A compound is defined herein as being “selective for the MC-3 receptor” when the above mentioned ratio “MC-3-selectivity” is at least about 10, preferably at least about 100, and more preferably at least about 500.

A compound is defined herein as being “selective for the MC-4 receptor” when the above mentioned ratio “MC-4-selectivity” is at least about 10, preferably at least about 100, and more preferably at least about 500.

### V. Methods of use and Compositions

Based on their ability to agonize or antagonize the MC-4 and/or MC-3 receptor, the present invention also relates to the use of the ligands described herein in methods for treating obesity and other body weight disorders, including, for example, anorexia and cachexia. The compounds may also be used in methods for treating disorders that result from body weight disorders, including but not limited to insulin resistance, glucose intolerance, Type-2 diabetes mellitus, coronary artery disease, elevated blood pressure, hypertension, dyslipidaemia, cancer (e.g., endometrial, cervical, ovarian, breast, prostate, gallbladder, colon), menstrual irregularities, hirsutism, infertility, gallbladder disease, restrictive lung disease, sleep apnea, gout, osteoarthritis, and thromboembolic disease. The invention further relates to the treatment of disorders relating to behavior, memory (including learning), cardiovascular function, inflammation, sepsis, cardiogenic and hypovolemic shock, sexual dysfunction, penile erection, muscle atrophy, nerve growth and repair, intrauterine fetal growth, and the like.

The terms treating and treatment are used herein to mean that, at a minimum, administration of a compound of the present invention mitigates a disorder by acting via the MC-3 or MC-4 receptor. Thus, the terms include: preventing a disease state from occurring in a mammal, particularly when the mammal is predisposed to acquiring the disease, but has not yet been diagnosed with the disease; inhibiting progression of the disease state; and/or alleviating or reversing the disease state.

The invention compounds can therefore be formulated into pharmaceutical compositions for use in treatment or prophylaxis of these conditions. Standard pharmaceutical formulation techniques are used, such as those disclosed in Reminigton's Pharmaceutical Sciences, Mack Publishing Company, Easton, Pa., latest edition and Peptide and Protein Drug Delivery, Marcel Dekker, N.Y., [1991.

The compositions of the invention comprise:
a. a safe and effective amount of a compound of Formula (1); and
b. a pharmaceutically-acceptable excipient.

A “safe and effective amount” of a Formula (I) compound is an amount that is effective to interact with the MC-4 and/or MC-3 receptor, in an animal, preferably a mammal, more preferably a human subject, without undue adverse side effects (such as toxicity, irritation, or allergic response), commensurate with a reasonable benefit/risk ratio when used in the manner of this invention. The specific “safe and effective amount” will, obviously, vary with such factors as the particular condition being treated, the physical condition of the patient, the duration of treatment, the nature of concurrent therapy (if any), the specific dosage form to be used, the excipient employed, the solubility of the Formula (I) compound therein, and the dosage regimen desired for the composition.

In addition to the subject compound, the compositions of the subject invention contain one or more pharmaceutically-acceptable excipients. The term “pharmaceutically-acceptable excipient”, as used herein, means one or more compatible solid or liquid ingredients which are suitable for administration to an animal, preferably a mammal, more preferably a human. The term “compatible”, as used herein, means that the components of the composition are capable of being commingled with the subject compound, and with each other, in a manner such that there is no interaction which would substantially reduce the pharmaceutical efficacy of the composition under ordinary use situations. Pharmaceutically-acceptable excipients must, of course, be of sufficiently high purity and sufficiently low toxicity to render them suitable for administration to the animal, preferably a mammal, more preferably a human being treated.

Some examples of substances which can serve as pharmaceutically-acceptable excipients or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; vegetable oils, such as peanut oil, cottonseed oil, sesame oil, olive oil, corn oil and oil of theobroma; polyols such as propylene glycol, glycerin, sorbitol, mannitol, and polyethylene glycol; agar; alginic acid; wetting agents and lubricants, such as sodium lauryl sulfate; coloring agents; flavoring agents; tableting agents, stabilizers; antioxidants; preservatives; pyrogen-free water; isotonic saline; and buffers, such as phosphate, citrate and acetate.

The choice of pharmaceutically-acceptable excipients to be used in conjunction with the subject compound is basically determined by the way the compound is to be administered. If the subject compound is to be injected, the preferred pharmaceutically-acceptable excipient is sterile water, physiological saline, or mixtures thereof, the pH of which has preferably been adjusted to about 4-10 with a pharmaceutical buffer; a compatible suspending agent may also be desirable.

In particular, pharmaceutically-acceptable excipients for systemic administration include sugars, starches, cellulose and its derivatives, malt, gelatin, talc, calcium sulfate, lactose, vegetable oils, synthetic oils, polyols, alginic acid, phosphate, acetate and citrate buffer solutions, emulsifiers, isotonic saline, and pyrogen-free water. Preferred excipients for parenteral administration include propylene glycol, ethyl oleate, pyrrolidone, ethanol, and sesame oil. Preferably, the pharmaceutically-acceptable excipient, in compositions for parenteral administration, comprises at least about 90% by weight of the total composition.

The compositions of this invention are preferably provided in unit dosage form. As used herein, a “unit dosage form” is a composition of this invention containing an amount of a Formula (I) compound that is suitable for administration to an animal, preferably a mammal, more preferably a human subject, in a single dose, according to good medical prac- tice. These compositions preferably contain from about 1 mg to about 750 mg, more preferably from about 3 mg to about 500 mg, still more preferably from about 5 mg to about 300 mg, of a Formula (I) compound.

The compositions of this invention may be in any of a variety of forms, suitable (for example) for oral, rectal, topical, nasal, ocular, transdermal, pulmonary or parenteral administration. Depending upon the particular route of administration desired, a variety of pharmaceutically-acceptable excipients well-known in the art may be used. These include solid or liquid fillers, diluents, hydrotropes, surface-active agents, and encapsulating substances. Optional pharmaceutically-active materials may be included, which do not substantially interfere with the inhibitory activity of the Formula (I) compound. The amount of excipient employed in conjunction with the Formula (I) compound is sufficient to provide a practical quantity of material for administration per unit dose of the compound. Techniques and compositions for making dosage forms useful in the methods of this invention are described in the following references, all incorporated by reference herein: Modern Pharmaceutics, Chapters 9 and 10 (Banker & Rhodes, editors, 1979); Lieberman et al., Pharmaceutical Dosage Forms: Tablets (1981); and Ansel, Introduction to Pharmaceutical Dosage For is 2d Edition (1976).

Various oral dosage forms can be used, including such solid forms as tablets, capsules, granules and bulk powders. These oral forms comprise a safe and effective amount, usually at least about 5%, and preferably from about 25% to about 50%, of the Formula (I) compound. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable excipient suitable for the preparation of unit dosage forms for peroral administration are well-known in the art. Tablets typically comprise conventional pharmaceutically-compatible adjuvants as inert diluents, such as calcium carbonate, sodium carbonate, mannitol, lactose and cellulose; binders such as starch, gelatin, polyvinylpyrrolidone and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of excipient components depends on secondary considerations like taste, cost, and shelf stability, which are not critical for the purposes of the subject invention, and can be readily made by a person skilled in the art.

Peroral compositions also include liquid solutions, emulsions, suspensions, and the like. The pharmaceutically-acceptable excipients suitable for preparation of such compositions are well known in the art. Typical components of excipients for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, Avicel® RC-591, tragacanth and sodium alginate; typical wetting agents include lecithin and polysorbate 80; and typical preservatives include methyl paraben, propyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit® coatings, waxes and shellac.

Because the compounds of the present invention are peptidic in nature, a preferred mode of administration is parenteral (more preferably intravenous injection) or nasal administration, in the form of a unit dose form. Preferred unit dose forms include suspensions and solutions, comprising a safe and effective amount of a Formula I compound. When administered parenterally, the unit dose form will typically comprise from about 1 mg to about 3 g, more typically from about 10 mg to about 1 g, of the Formula (I) compound, although the amount of compound administered will depend, for example, on its relative affinity for the MC-4/MC-3 receptor subtypes, its selectivity over other receptors, including the other melanocortin receptors, etc.

Compositions of the subject invention may optionally include other drug actives.

Other compositions useful for attaining systemic delivery of the subject compounds include sublingual and buccal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

### VI. Methods of Administration

As indicated, compositions of this invention can be administered topically or systemically. Systemic application includes any method of introducing a Formula (I) compound into the tissues of the body, e.g., intra-articular, intrathecal, epidural, intramuscular, transdermal, intravenous, intr-aperitoneal, subcutaneous, sublingual, rectal, nasal, pulmonary, and oral administration. The Formula (I) compounds of the present invention are preferably administered systemically, more preferably parenterally and most preferably via intravenous injection.

The specific dosage of compound to be administered, as well as the duration of treatment, and whether the treatment is topical or systemic are interdependent. The dosage and treatment regimen will also depend upon such factors as the specific Formula (I) compound used, the treatment indication, the personal attributes of the subject (such as weight), compliance with the treatment regimen, and the presence and severity of any side effects of the treatment.

Typically, for a human adult weighing approximately 70 kilograms, from about 1 mg to about 6 g, more typically from about 100 mg to about 3 g, of Formula (I) compound are administered per day for systemic administration. It is understood that these dosage ranges are by way of example only, and that daily administration can be adjusted depending on the factors listed above.

As is known and practiced in the art, all formulations for parenteral administration must be sterile. For mammals, especially humans, (assuming an approximate body weight of 70 kilograms) individual doses of from about 0.001 mg to about 100 mg are preferred.

A preferred method of systemic administration is intravenous delivery. Individual doses of from about 0.01 mg to about 100 mg, preferably from about 0.1 mg to about 100 mg are preferred when using this mode of delivery.

In all of the foregoing, of course, the compounds of the invention can be administered alone or as mixtures, and the compositions may further include additional drugs or excipients as appropriate for the indication.

The compound of the invention can be delivered to the preferred site in the body by using a suitable drug delivery system. Drug delivery systems are well known in the art. For example, a drug delivery technique useful for the compounds of the present invention is the conjugation of the compound to an active molecule capable of being transported through a biological barrier (see e.g. Zlokovic, B. V., Pharmaceutical Research, Vol. 12, pp. 1395-1406 (1995)). A specific example constitutes the coupling of the compound of the invention to fragments of insulin to achieve transport across the blood brain barrier (Fukuta, M., et al. Pharmaceutical Res., Vol. 11, pp. 1681-1688 (1994)). For general reviews of technologies for drug delivery suitable for the compounds of the invention see Zlokovic, B. V., Pharmaceutical Res., Vol. 12, pp. 1395-1406 (1995) and Pardridge, W M, Pharmacol. Toxicol., Vol. 71, pp. 3-10 (1992).

### VII. Representative Synthetic Examples

In the following examples, the invention will be described in greater detail by reference to a number of preferred embodiments which are only given for purposes of illustration and should not be considered to limit the invention in any way.

The following abbreviations are used in the Examples:

| | |
|---|---|
| Ac: acetyl [—C(O)CH₃] | Aun: aminoundecanoic |
| Atc: (D,L)-2-aminotetraline-2- | |
| carboxylic acid | |
| Bc: butanoyl [—C(O)(CH₂)₂CH₃] | Boc: tert-butyloxycarbonyl |
| DCM: dichioromethane | DEA: diethylamine |
| DMF: N,N-dimethylformamide | DMAP: 4-dimethylaminopyridine |
| DME: 1,2-dimethoxyethane | DIEA: diisopropylethylamine |
| DPPA: Diphenyiphosphoryl azide | EtOAc: ethyl acetate |

EDCI: 1-ethyl-3-(3′-dimethylaminopropyl)carbodiimide hydrochloride

| | |
|---|---|
| Fmoc: 9-Fluorenylmethoxycarbonyl | |
| HOBt: N-hydroxybenzotriazole, | |
| monohydrate | |
| HOAt: 1-hydroxy-7-azabenzotriazole | i-PrOH: 2-propanol |
| MeOH: methanol | NMM: N-methylmorpholine |
| OtBu: tert-butoxy [—O—C(CH₃)₃] | |
| Pbf: 2,2,4,6,7-pentamethyl- | |
| dihydrobenzofurane-5-sulfonyl- | |
| Pmc: 2,2,5,7,8-pentamethyl-6- | |
| chromansulfonyl- | |
| p-TSA: p-toluenesulfonate | |

PyBOP: benzotriazole-1yl-oxy-tris-pyrrolidinophosphonium
hexafluorophosphate
PyBroP: bromo-tris-pyrrolidino-phosphonium hexafluorophosphate

| | |
|---|---|
| tBu: tert-butyl [—C(CH₃)₃] | TEA: triethylamine |
| TFA: trifluoroacetic acid | THF: tetrahydrofuran |

### EXAMPLE 1

### Synthesis of Ac—YfRW—NH₂

Based on the 0.55 mmole/g substitution rate for the Rink Amide resin, 0.45 g of the resin is weighted out for 0.25 mmole scale synthesis. The performance of the PE-ABD 433 peptide synthesizer is checked before the run with various flow tests to ensure the accurate reagent delivery. Fmoc amino acids: Tyr-OtBu, Arg-Pmc, and Trp-Boc are purchased commercially in 1 mmole cartridges. Fmoc-phe (387 mg, 1 mmole) is measured and added in the synthesis cartridges. The freshly made acetic anhydride solution is loaded on the instrument at #4 bottle position. Other synthesis reagents and solvents are purchased commercially and loaded on the instrument according to the instrument's instruction. A chemistry program called NAc-0.25 mmole MonPrePk is used for synthesizing this peptide. The Fmoc deprotection is monitored and controlled by conductivity measurement with set criteria of 5% or less conductivity comparing to previous deprotection cycle.

The resin is air-dried and transferred into a glass vial and a freshly prepared cleavage reagent (10 ml) is added. The deprotection reaction is carried out for 2 hours at room temperature with constant stirring. The supernatant is then separated from the resin by filtration. The synthesized peptide is then precipitated in ether layer by adding 40 ml cold ether. The peptide precipitates are centrifuged (Heraeus Labofuge 400, Rotor #8179) at 3,500 rpm for four minutes. The ether is discarded and 40 ml of fresh cold ether is added to wash the peptide precipitates. The washing steps are repeated for three times to remove the deprotection byproducts. The final peptide precipitates are freeze-dried overnight. The identity and purity of the linear peptide is determined by both MS and HPLC. Expected peptide molecular weight is detected.

Peptide is re-dissolved in 50% acetic acid and purified by a C8 reverse phase HPLC using a linear gradient of 0-70% solvent B with solvent A in 70 min at a flow rate 3 ml/min. The composition of solvents A and B are as follows: A: 0.1% TFA, 2% acetonitrile in water; B: 0.1% TFA in 95% acetonitrile. The fractions are collected at every 0.25 min. Aliquots of each fraction are analyzed by both MS and analytical RP-UPLC. The fractions that contain a single u.v. 220 nm absorbance peak with expected mass unit for the peptide are combined and lyophilized. The final purity of the peptide is determined by an analytical RP-HPLC of the combined fractions.

The peptides described in Examples 2-54 below are readily synthesized according to the same protocol as Example l, but with the modifications as noted.

### EXAMPLE 2

### Synthesis of Ac—YFRW—NH₂

Prepared according to Example 1, except Fmoc-L-Phe is used instead Fmoc-D-Phe.

### EXAMPLE 3

### Synthesis of Ac-FfRW—NH₂

Prepared according to Example 1, except Fmoc-L-Phe is used instead Fmoc-L-Tyr(OtBu).

### EXAMPLE 4

### Synthesis of Ac-PFRW—NH₂

Prepared according to Example 1, except Fmoc-L-Pro is used instead Fmoc-L-Tyr(OtBu).

### EXAMPLE 5

### Synthesis of Ac-AfRW—NH₂

Prepared according to Example 1, except Fmoc-L-Ala is used instead Fmoc-L-Tyr(OtBu).

### EXAMPLE 6

### Synthesis of Ac-(2-Nal)fRW—NH₂

Prepared according to Example 1, except Fmoc-L-(2-Nal) is used instead Fmoc-L-Tyr(OtBu).

### EXAMPLE 7

### Synthesis of Ac—YfR(2-Nal)-NH₂

Prepared according to Example 1, except Fmoc-L-(2-Nal) is used instead Fmoc-L-Trp(Boc).

### EXAMPLE 8

### Synthesis of Ac—YfR(1-Nal)-NH₂

Prepared according to Example 1, except Fmoc-L-(1-Nal) is used instead Fmoc-L-Trp(Boc).

### EXAMPLE 9

### Synthesis of Ac—YfHW—NH₂

Prepared according to Example 1, except Fmoc-L-His(Trt) is used instead Fmoc-L-Arg(Pmc).

### EXAMPLE 10

### Synthesis of Ac—Y(D-2-Nal)RW—NH₂

Prepared according to Example 1, except Fmoc-D-(2-Nal) is used instead Fmoc-D-Phe.

### EXAMPLE 11

### Synthesis of Ac—Y(L-N-Me-Phe)RW—NH₂

Prepared according to Example 1, except Fmoc-L-N-Me-Phe is used instead Fmoc-D-Phe.

### EXAMPLE 12

### Synthesis of Ac-A(D-N-Me-Phe)RW—NH₂

Prepared according to Example 1, except Fmoc-D-N-Me-Phe is used instead Fmoc-L-Phe, and Fmoc-L-Ala is used instead of Fmoc-L-Tyr(OtBu).

### EXAMPLE 13

### Synthesis of Ac—YF(L-N-Me-Arg)W—NH

Prepared according to Example 1, except Fmoc-L-N-Me-Arg(Mtr) is used instead Fmoc-L-Arg(Pmc), and Fmoc-L-Phe is used instead of Fmoc-D-Phe.

### EXAMPLE 14

### Synthesis of Ac—Yf(L-N-Me-Arg)W—NH₂

Prepared according to Example 1, except Fmoc-L-N-Me-Arg(Mtr) is used instead Fmoc-L-Arg(Pmc).

### EXAMPLE 15

### Synthesis of Ac-(L-N-Me-Tyr)FRW—NH₂

Prepared according to Example 1, except Fmoc-L-N-Me-Tyr(Bzl) is used instead Fmoc-L-Tyr(OtBu), and Fmoc-L-Phe is used instead of Fmoc-D-Phe.

### EXAMPLE 16

### Synthesis of Ac-(L-N-Me-Tyr)fRW—NH₂

Prepared according to Example 1, except Fmoc-L-N-Me-Tyr(Bzl) is used instead Fmoc-L-Tyr(OtBu).

### EXAMPLE 17

### Synthesis of Ac—Y(D-4-Chloro-Phe)RW—NH₂

Prepared according to Example 1, except Fmoc-D-4-Chloro-Phe is used instead Fmoc-D-Phe.

### EXAMPLE 18

### Synthesis of Ac—Y(D-4-Fluoro-Phe)RW—NH₂

Prepared according to Example 1, except Fmoc-D-4-Fluoro-Phe is used instead Fmoc-D-Phe.

### EXAMPLE 19

### Synthesis of Ac—Y(D-3,4-Dichloro-Phe)RW—NH₂

Prepared according to Example 1, except Fmoc-D-3,4-Dichloro-Phe is used instead Fmoc-D-Phe.

### EXAMPLE 20

### Synthesis of Ac—Y(D-4-Me-Phe)RW—NH₂

Prepared according to Example 1, except Fmoc-D-4-Me-Phe is used instead Fmoc-D-Phe.

### EXAMPLE 21

### Synthesis of Ac—Y(D-4-Nitro-Phe)RW—NH₂

Prepared according to Example 1, except Fmoc-D-4-Nitro-Phe is used instead Fmoc-D-Phe.

### EXAMPLE 22

### Synthesis of Ac—Y(D-Phenylglycine)RW—NH₂

Prepared according to Example 1, except Fmoc-D-Phenylglycine is used instead Fmoc-D-Phe.

### EXAMPLE 23

### Synthesis of Ac—Y(D-4-Homo-Phe)RW—NH₂

Prepared according to Example 1, except Fmoc-D-4-Homo-Phe is used instead Fmoc-D-Phe.

### EXAMPLE 24

### Synthesis of Ac—Y(D-Strylryalanine)RW—NH₂

Prepared according to Example 1, except Fmoc-D-Strylryalanine is used instead Fmoc-D-Phe.

### EXAMPLE 25

### Synthesis of Ac—Y(D-4-Thienylalanine)RW—NH₂

Prepared according to Example 1, except Fmoc-D-4-Thienylalanine is used instead Fmoc-D-Phe.

### EXAMPLE 26

### Synthesis of Ac—Y(D-3-Fluoro-Phe)RW—NH₂

Prepared according to Example 1, except Fmoc-D-3-Fluoro-Phe is used instead Fmoc-D-Phe.

### EXAMPLE 27

### Synthesis of Ac-(L-4-Fluoro-Phe)(D-4-Fluoro-Phe)RW—NH₂

Prepared according to Example 1, except Fmoc-L-4-Fluoro-Phe is used instead Fmoc-L-Tyr(OtBu), and Fmoc-D-4-Fluoro-Phe is used instead of Fmoc-D-Phe.

### EXAMPLE 28

### Synthesis of Ac—Y(D-2-Fluoro-Phe)RW—NH₂

Prepared according to Example 1, except Fmoc-D-2-Fluoro-Phe is used instead Fmoc-D-Phe.

### EXAMPLE 29

### Synthesis of Ac-(L-4-Chloro-Phe)(D-4-Fluoro-Phe)RW—NH₂

Prepared according to Example 1, except Fmoc-L-4-Chloro-Phe is used instead Fmoc-L-Tyr(OtBu), and Fmoc-D-4-Fluoro-Phe is used instead of Fmoc-D-Phe.

### EXAMPLE 30

### Synthesis of Ac-(L-4-Chloro-Phe)(D-4-Fluoro-Phe)RW(N-Me-Gly)-NH₂

Prepared according to Example 1, except Fmoc-L-4-Chloro-Phe is used instead Fmoc-L-Tyr(OtBu), Fmoc-D-4-Fluoro-Phe is used instead of Fmoc-D-Phe, and an additional Fmoc-N-Me-Gly is used.

### EXAMPLE 31

### Synthesis of Ac-(L-4-Chloro-Phe)fRW(N-Me-Gly)-NH₂

Prepared according to Example 1, except Fmoc-L-4-Chloro-Phe is used instead Fmoc-L-Tyr(OtBu), and an additional Fmoc-N-Me-Gly is used.

### EXAMPLE 32

### Synthesis of Ac-(L-4-Chloro-Phe)(D-4-Fluoro-Phe)R—NH₂

Prepared according to Example l, except Fmoc-L-4-Chloro-Phe is used instead Fmoc-L-Tyr(OtBu), Fmoc-D-4-Fluoro-Phe is used instead of Fmoc-D-Phe, and Fmoc-L-Trp(Boc) is not used.

### EXAMPLE 33

### Synthesis of Ac-(L-4-Chloro-Phe)(D-4-Fluoro-Phe)RG-NH₂

Prepared according to Example 1, except Fmoc-L-4-Chloro-Phe is used instead Fmoc-L-Tyr(OtBu), Fmoc-D-4-Fluoro-Phe is used instead of Fmoc-D-Phe, and Fmoc-L-Gly is used instead of Fmoc-L-Trp(Boc).

### EXAMPLE 34

### Synthesis of Ac-(L-3,4-Difluoro-Phe)fRW—NH₂

Prepared according to Example 1, except Fmoc-L-3,4-Difluoro-Phe is used instead Fmoc-L-Tyr(OtBu).

### EXAMPLE 35

### Synthesis of Ac—Y(D-2-Me-Phe)RW—NH₂

Prepared according to Example 1, except Fmoc-D-2-Me-Phe is used instead Fmoc-D-Phe.

### EXAMPLE 36

### Synthesis of Ac-(L-4-Bromo-Phe)fRW—NH₂

Prepared according to Example 1, except Fmoc-L-4-Bromo-Phe is used instead Fmoc-L-Tyr(OtBu).

### EXAMPLE 37

### Synthesis of Ac-(L-4-Iodo-Phe)fRW—NH₂

Prepared according to Example 1, except Fmoc-L-4-Iodo-Phe is used instead Fmoc-L-Tyr(OtBu).

### EXAMPLE 38

### Synthesis of Ac-(L-Pentafluoro-Phe)fRW—NH₂

Prepared according to Example 1, except Fmoc-L-Pentafluoro-Phe is used instead Fmoc-L-Tyr(OtBu).

### EXAMPLE 39

### Synthesis of Ac-(L-4-Nitro-Phe)fRW—NH₂

Prepared according to Example 1, except Fmoc-L-4-Nitro-Phe is used instead Fmoc-L-Tyr(OtBu).

### EXAMPLE 40

### Synthesis of Ac-(L-Aminomethyl-Phe)fRW—NH₂

Prepared according to Example 1, except Fmoc-L-Aminomethyl-Phe(Boc) is used instead Fmoc-L-Tyr(OtBu).

### EXAMPLE 41

### Synthesis of Ac-(L-Tetraisoquinoline-3-carboxylic acid)fRW—NH₂

Prepared according to Example 1, except Fmoc-L-Tetraisoquinoline-3-carboxylic acid is used instead Fmoc-L-Tyr(OtBu).

### EXAMPLE 42

### Synthesis of Ac-(L-Homo-Phe)fRW—NH₂

Prepared according to Example 1, except Fmoc-L-Homo-Phe is used instead Fmoc-L-Tyr(OtBu).

### EXAMPLE 43

### Synthesis of Ac-(L-Biphenyl-Alanine)fRW—NH₂

Prepared according to Example 1, except Fmoc-L-Biphenyl-Alanine is used instead Fmoc-L-Tyr(OtBu).

### EXAMPLE 44

### Synthesis of Ac-(L-4-SO₃-Phe)fRW—NH₂

Prepared according to Example 1, except Fmoc-L-4-SO₃-Phe is used instead Fmoc-L-Tyr(OtBu).

### EXAMPLE 45

### Synthesis of Ac-(L-2,6-Dimethyl-Phe)fRW—NH₂

Prepared according to Example 1, except Fmoc-L-2,6-Dimethyl]-Phe is used instead Fmoc-L-Tyr(OtBu).

### EXAMPLE 46

### Synthesis of Ac-(L-4-Methyl-Phe)fRW—NH₂

Prepared according to Example 1, except Fmoc-L-4-Methyl-Phe is used instead Fmoc-L-Tyr(OtBu).

### EXAMPLE 47

### Synthesis of Ac-(L-4-NH-Phe)fRW—NH₂

Prepared according to Example 1, except Fmoc-L-4-NH-Phe(Boc) is used instead Fmoc-L-Tyr(OtBu).

### EXAMPLE 48

### Synthesis of Ac—YfKW—NH₂

Prepared according to Example 1, except Fmoc-L-Lys(Boc) is used instead Fmoc-L-Arg(Pmc).

### EXAMPLE 49

### Synthesis of Ac—Yf(Orn)W—NH₂

Prepared according to Example 1, except Fmoc-L-Orn(Boc) is used instead Fmoc-L-Arg(Pmc).

### EXAMPLE 50

### Synthesis of Bc-HFRW(N-Me-Gly)-NH₂

Prepared according to Example 1, except Fmoc-L-His(Trt) is used instead Fmoc-L-Tyr(OtBu), Fmoc-L-Phe is used instead of Fmoc-D-Phe, butyric anhydride is used instead of acetic anhydride, and an additional Fmoc-L-N-Me-Gly is used.

### EXAMPLE 51

### Synthesis of Bc-HfRW(N-Me-Gly)-NH₂

Prepared according to Example 1, except Fmoc-L-His(Trt) is used instead Fmoc-L-Tyr(OtBu), butyric anhydride is used instead of acetic anhydride, and an additional Fmoc-L-N-Me-Gly is used.

### EXAMPLE 52

### Synthesis of Bc-YfRW(N-Me-Gly)-NH₂

Prepared according to Example 1, except butyric anhydride is used instead of acetic anhydride, and an additional Fmoc-L-N-Me-Gly is used.

### EXAMPLE 53

### Synthesis of Bc-YFRW(N-Me-Gly)-NH₂

Prepared according to Example 1, except Fmoc-L-Phe is used instead of Fmoc-D-Phe, butyric anhydride is used instead of acetic anhydride, and an additional Fmoc-L-N-Me-Gly is used.

### EXAMPLE 54

### Synthesis of Bc-FfRW(N-Me-Gly)-NH₂

Prepared according to Example 1, except Fmoc-L-Phe is used instead Fmoc-L-Tyr(OtBu), butyric anhydride is used instead of acetic anhydride, and an additional Fmoc-L-N-Me-Gly is used.

B. Solution Phase Chemistry

### EXAMPLE 55

### Synthesis of 5-Guanidino-2-[3-phenyl-2-(3-phenyl-propylamino)-propionylamino]1-pentanoic acid (naphthalene-1-ylmethyl)-amide

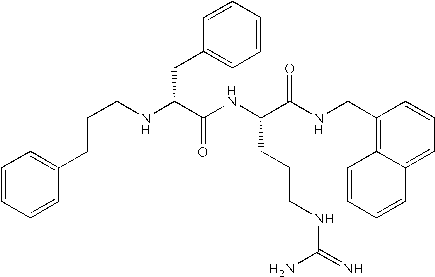

### 2-(S)-(2-(R)-tert-Butoxycarbonylamino-3-phenyl-propionylamino)-5-nitroguanidino-pentanoic acid methyl ester

To a solution of 2-(R)-(2-tert-butoxycarbonylamino-3-phenyl-propionic acid (2.0 g, 7.54 mmoles) and 2-(S)-amino-5-nitroguanidino-pentanoic acid methyl ester (2.0 g, 1.1 eq) in 100 ml of anhydrous DMF is added 1-[3-(dimethylamino)propyl)-3-ethylcarbodiimide hydrochloride (1.5 g, 1.5 eq), 1-hydroxybenzotriazole (2.16 g, 1.4 eq) and triethylamine (3.0 ml, 3 eq). The resulting solution is stirred at room temperature for 20 hours, and then the solvents are removed under reduced pressure. The resulting residue is partitioned between 10% sodium carbonate (100 ml) and methylene chloride (100 ml). The organics are dried over anhydrous magnesium sulfate, filtered and the solvent removed under reduced pressure. The resulting crude material is purified by flash chromatography on silica gel (90:9:1 chloroform:methanol:ammonium hydroxide) to afford the title compound.

### 2-(S)-(2-(R)-tert-butoxycarbonylamino-3-phenyl-propionylamino)-5-nitroguanidino-pentanoic acid

To a solution of 2-(S)-(2-(R)-tert-butoxycarbonylamino-3-phenyl-propionylamino)-5-nitroguanidino-pentanoic acid methyl ester (3.6 g, 7.5 mmoles) in tetrahydrofuran (100 ml) is added lithium hydroxide monohydrate (387 mg, 1.2 eq) and water (10 ml). After stirring at room temperature for one hour, the reaction is neutralized with trifluoroacetic acid (0.7 ml) and the solvents are removed under reduced pressure. The resulting residue is partitioned between water (200 ml) and ethyl acetate (200 ml). The aqueous layer is extracted with ethyl acetate (3×250 ml), the organics are pooled and dried over anhydrous magnesium sulfate, filtered and the solvent removed under reduced pressure. The crude material is used without further purification.

### (1-(S)-{4-Nitroguanidino-1-[naphthalen-1-yl-methyl)-carbamoyl]-butylcarbamoyl}-2-(R)-phenyl-ethyl)-carbamic acid tert-butyl ester

To a solution of 2-(S)-(2-(R)-tert-butoxycarbonylamino-3-phenyl-propionylamino)-5-guanidino-pentanoic acid (1.0 g, 2.15 mmoles) and C-naphthalen-1-yl-methylamine (0.377 ml, 1.2 eq) in 50 ml of anhydrous DMF is added 1-[3-(dimethylamino)propyl)-3-ethylcarbodiimide hydrochloride (532 mg, 1.5 eq), 1-hydroxybenzotriazole (376, 1.4 eq) and triethylamine (0.9 ml, 3 eq). The resulting solution is stirred at room temperature for 20 hours, and then the solvents are removed under reduced pressure. The resulting residue is partitioned between 10% sodium carbonate (75 ml) and chloroform (75 ml). The organics are dried over anhydrous magnesium sulfate, filtered and the solvent removed under reduced pressure. The resulting crude material is purified by flash chromatography on silica gel (90:9:1 chloroform:methanol:ammoniumhydroxide) to afford the title compound.

### 2-(S)-2-(R)-Amino-3-phenyl-propionylamino)-5-nitroguanidino-pentanoic acid (naphthalen-1-ylmethyl)-amide trifluoroacetic acid salt

To a solution of (1-(S)-{4-nitroguanidino-1-[naphthalen-1-ylmethyl-carbamoyl]-butylcarbamoyl}-2-(R)-phenyl-ethyl)-carbamic acid tert-butyl ester (1.1 g, 1.82 mmoles) in methylene chloride (100 ml) is added trifluoroacetic acid (50 ml). The resulting solution is stirred at room temperature for three hours and the then the solvents are removed under reduced pressure. The crude material is purified by reverse phase preparative HPLC to afford the title compound.

### 5-Nitroguanidino-2-[3-phenyl-2-(3-phenyl-propylamino)-propionylamino]-pentanoic acid(naphthalen-1-ylmethyl)-amide

To a suspension of 2-(S)-(2-(R)-amino-3-phenyl-propionylamino)-5-guanidino-pentanoic acid (naphthalen-1-ylmethyl)-amide (750 mg, 1.21 mmoles), 3-phenyl-propionaldehyde (0.159 ml, 1.0 eq), and activated molecular sieves (4 angstrom, crushed) is added triethylamine (0.247 ml, 1.5 eq). The resulting suspemsion is stirred at room temperature for 24 hours, and then the pH is adjusted to 5 with acetic acid. A 1.0M solution of sodium cynanoborohydride in tetrahydrofuran (1.44 mi, 1.2 eq) is then added at a rate of 0.2 ml/min with a syringe pump. The resulting suspension is stirred at room temperature for 24 hours, filtered through celite, and the solvents removed under reduced pressure. The crude material is purified by reverse-phase HPLC to afford the title compound.

### 5-Guanidino-2-[3-phenyl-2-(3-phenyl-propylamino)-propionylamino]l-pentanoic acid(naphthalen-1-ylmethyl)-amide

To a solution of 5-nitroguanidino-2-[3-phenyl-2-(3-phenyl-propylamino)-propionylamino]-pentanoic acid (naphthalen-1-ylmethyl)-amide (140 mg, 0.165 mmoles) in methanol (30 ml) is added acetic acid (3 ml) and 5% palladium on barium sulfate (100 mg). The resulting suspension is hydrogenated at atmospheric pressure for twenty-four hours, and then filtered through celite. The solvents are removed under reduced pressure and the crude product purified by reverse-phase HPLC.

### EXAMPLE 56

### Synthesis of 5-Guanidino-2-(S)-[3-phenyl-2-(R)-propylamino)-propionylamino]l-pentanoic acid (2-naphthalen-2-yl-ethyl)-amide

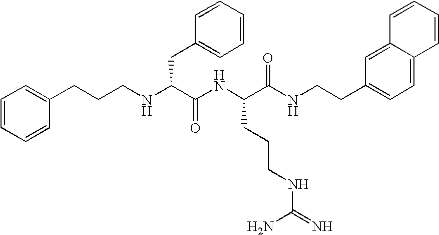

### Toluene-4-sulfonic acid 2-naphthalen-2yl-ethyl ester

To a solution of 2-naphthalen-2yl-ethanol (3.0 g, 17.4 mmoles) in tetrahydrofuran (50 ml), is added para-toluenesulfonic anhydride (6.8 g, 1.2 eq), and triethylamine (7.1 ml, 3 eq). The resulting solution is stirred at room temperature for one hour and then the solvents are removed under reduced pressure. The crude material is purified by flash chromatography on silica gel (20% ethyl acetate/hexane) to afford the title compound.

### 2-(2-Azido-ethyl)-naphthalene

To a solution of toluene-4-sulfonic acid 2-naphthalen-2yl-ethyl ester (5.0 g, 15.3 mmoles) in DMF (100 ml) is added sodium azide (1.3 g, 1.3 eq). The resulting suspension is heated to 80° C. for twenty-four hours and then cooled to room temperature. The solvent is removed under reduced pressure and the residue partitioned between ethyl acetate (200 ml) and water (200 ml). The organics are dried over anhydrous magesium sulfate, filtered and the solvent removed under reduced pressure to afford the title compound. The crude material is used without further purification.

### 2-Naphthalen-2-yl-ethylamine

To a solution of 2-(2-azido-ethyl)-naphthalene (3.0 g, 15.2 mmoles), in tetrahydrofuran (100 ml) is added triphenylphosphine (6.0 g, 1.5 eq) and water (5 ml). The resulting solution is heated to reflux for three hours and then cooled to room temperature. The solvents are removed under reduced pressure and the crude material purified by flash chromatography on silica gel (90:9:1 chloroform:methanol:ammonium hydroxide). The purified material is then converted to the trifluoroacetic acid salt by the addition of excess trifluoroacetic acid, and subsequent removal of the excess acid by evaporation under reduced pressure to afford the title compound.

### {1-(S)-[4-Nitroguanidino-1-(2-naphthalen-2-yl-ethylcarbamoyl)-butylcarbamoyl]-2-(R)-phenyl-ethyl}-carbamic acid tert-butyl ester

To a solution of 2-(S)-(2-(R)-tert-butoxycarbonylamino-3-phenyl-propionylamino)-5-nitroguanidino-pentanoic acid (1.0 g, 2.144 mmoles) and 2-(2-azido-ethyl)-naphthalene (733 mg, 1.2 eq) in DMF (50 ml) is added 1-[3-(dimethylamino)propyl)-3-ethylcarbodiimide hydrochloride (614 mg, 1.5 eq), 1-hydroxybenzotriazole (434 mg, 1.5 eq), and triethylamine (0.877 ml, 3 eq). The resulting suspension is stirred at room temperature for twenty-four hours, and the solvents are then removed under reduced pressure. The crude material is partitioned between 10% sodium carbonate (75 ml) and methylene chloride (75 ml). The organics are dried over anhydrous magnesium sulfate, filtered and solvents removed under reduced pressure. The crude material is purified by flash chromatography on silica gel (90:9:1 chloroform:methanol:ammonium hydroxide) to afford the title compound.

### 2-(S)-(2-(R)-Amino-3-phenyl-propionylamino)-5-nitroguanidino-pentanoic acid (2-naphthalen-2-yl-ethyl)-amide

To a solution of {1-(S)-[4-nitroguanidino-1-(2-naphthalen-2-yl-ethylcarbamoyl)-butylcarbamoyl]-2-(R)-phenyl-ethyl}-carbamic acid tert-butyl ester (1.5 g, 2.04 mmoles) in methylene chloride (100 ml) is added trifluoroacetic acid (50 ml). The resulting solution is stirred at room temperature for twenty-four hours and then the solvents are removed under reduced pressure. The crude material is purified by flash chromatography on silica gel (90:9:1 chloroform:methanol:ammonium hydroxide) to afford the title compound.

### 5-Nitroguanidino-2-(S)-[3-phenyl-2-(R)-propylamino)-propionylamino]-pentanoic acid (2-naphthalen-2-yl-ethyl)-amide

To a solution of 2-(S)-(2-(R)-amino-3-phenyl-propionylamino)-5-nitroguanidino-pentanoic acid (2-naphthalen-2-yl-ethyl)-amide (600 mg, 1.15 mmoles) in tetrahydrofuran (50 ml) is added 3-phenyl-propionaldehyde (0.121 ml, 0.8 eq) and molecular sieves (4 angstrom, crushed). The resulting suspension is stirred at room temperature for twenty-four hours and then the pH is adjusted to 5 with acetic acid. To this solution is added sodium cyanoborohydride (1.38 ml, 1.2 eq, 1.0M solution in tetrahydrofuran) at rate of 0.2 ml/hour using a syringe pump. The resulting suspension is stirred at room temperature for twenty-four hours, and then the solvents are removed under reduced pressure. The crude material is filtered and then purified by reverse- phase HPLC to afford the title compound.

### 5-Guanidino-2-(S)-[3-phenyl-2-(R)-propylamino)-propionylamino]-pentanoic acid (2-naphthalen-2-yl-ethyl)-amide

To a solution of 5-nitroguanidino-2-(S)-[3-phenyl-2-(R)-propylamino)-propionylamino]-pentanoic acid (2-naphthalen-2-yl-ethyl)-amide (294 mg, 0.46 mmoles) in 50 ml of methanol is added acetic acid (5 ml) and 5% palladium on barium sulfate (294 mg). The resulting suspension is hydrogenated at atmospheric pressure for twenty-four hours, filtered through celite, and the solvents removed under reduced pressure. The crude material is purified by reverse-phase HPLC to afford the title compound.

### EXAMPLE 57

### Synthesis of 5-Guanidino-2-(S)-[3-phenyl-2-(R)-(3-phenyl-propylamino)-propionylamino]-pentanoic acid (2-naphthalen-1-yl-ethyl)-amide

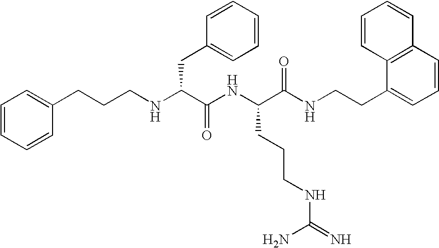

### Toluene-4-sulfonic acid 2-naphthalen-1-yl-ethyl ester

To a solution of 2-naphthalen-1-yl-ethanol (3.0 g, 17.4 mmoles) in tetrahydrofuran (50 ml) is added para-toluene sulfonic anhydride (6.8 g, 1.2 eq) and triethylamine (7.1 ml, 3 eq). The resulting solution is stirred at room temperature for one hour and then the solvents are removed under reduced pressure. The crude product is purified by flash chromatography on silica gel (20% ethyl acetate/hexanes) to afford the title compound.

### 1-(2-Azido-ethyl)-naphthalene

To a solution of toluene-4-sulfonic acid 2-naphthalen-1-yl-ethyl ester (5.2 g, 15.9 mmoles) in DMF (100 ml) is added sodium azide (1.3 g, 1.3 eq). The resulting solution is heated to 80° C. to twenty-four hours and then cooled to room temperature and the solvents removed under reduced pressure. The residue is partitioned between ethyl acetate and water, the organics are dried over anhydrous magnesium sulfate, filtered, and the solvents removed under reduced pressure to afford the title compound. The crude product is used without further purification.

### 2-Naphthalen-1-yl-ethylamine

To a solution of 1-(2-azido-ethyl)-naphthalene (3.0 g, 15.23 mmoles) in tetrahydrofuran (100 ml) is added triphenylphosphine (6.0 g, 1.5 eq) and water (5 ml). The resulting solution is refluxed for three hours and then cooled to room temperature. The solvents are removed under reduced pressure and the crude product is purified by flash chromatography on silica gel (90:9:1 chloroform:methanol:ammonium hydroxide) to afford the title compound. The addition of excess trifluoroacetic acid, followed by evaporation provided the trifluoroacetic acid salt.

### {1-(S)-[4-nitroguanidino-1-(2-naphthalen-1-yl-ethylcarbamoyl)-butylcarbamoyl]-2-(R)-phenyl-ethyl]-carbamic acid tert-butyl ester

To a solution of 2-(S)-(2-(R)-tert-butoxycarbonylamino-3-phenyl-propionylamino)-5-nitroguanidino-pentanoic acid methyl ester (1.0 g, 2.1 mmoles) and 2-naphthalen-1-yl-ethylamine (733 mg, 1.2 eq) in DMF (50 ml) is added 1-hydroxybenzotriazole (434 mg, 1.5 eq), 1-[3-(dimethylaminopropyl]-3-ethylcarbodiimide hydrochloride (614 mg, 1.5 eq), and triethylamine (0.877 ml, 3 eq). The resulting suspension is stirred at room temperature for twenty-four hours, and then the solvents are removed under reduced pressure. The residue is partitioned between methylene chloride and 10% sodium carbonate, the organics dried over anhydrous magnesium sulfate, filtered and the solvent removed under reduced pressure to give the crude product. The product is purified by flash chromatography on silica gel (90:9:1 chloroform:methanol:ammonium hydroxide) to afford the title compound.

### 2-(S)-(2-(R)-Amino-3-phenyl-propionylamino)-5-nitroguanidino-pentanoic acid (2-naphthalen-1-yl-ethyl)-amide

To a solution of {1-(S)-[4-nitroguanidino-1-(2-naphthalen-1-yl-ethylcarbamoyl)-butylcarbamoyl]-2-(R)-phenyl-ethyl]-carbamic acid tert-butyl ester (1.3 g, 1.77 mmoles) in methylene chloride (100 ml) is added trifluoroacetic acid (50 ml). After stirring at room temperature for twenty-four hours the solvents are removed under reduced pressure ant the residue partitioned between 10% sodium carbonate (100 ml) and ethyl acetate (100 ml). The organics are dried over anhydrous magnesium sulfate, filtered, and the solvents removed under reduced pressure. The crude product is purified by flash chromatography on silica gel (90:9:1 chloroform:methanol:ammonium hydroxide) to give the title compound.

### 5-Nitroguanidino-2-(S)-[3-phenyl-2-(R)-(3-phenyl-propylamino)-propionylamino]-pentanoic acid(2-naphthalen-1-yl-ethyl)-amide

To solution of 2-(S)-(2-(R)-amino-3-phenyl-propionylamino)-5-nitroguanidino-pentanoic acid (2-naphthalen-1-yl-ethyl)-amide (690 mg, 1.33 mmoles) in tetrahydrofuran (50 ml) is added 3-phenyl-propionaldehyde (0.14 ml, 0.8 eq) and molecular seives (500 mg, 4 A powdered). The resulting suspension is stirred at room temperature for twenty-four hours and then the pH is adjusted to 5 with acetic acid. Sodium cyanoborohydride (1.6 ml 1.2 eq 1.0M solution in tetrahydrofuran) is then slowly added (0.2 ml/hour) with a syringe pump. After twenty-four hours the solvents are removed under reduced pressure and the crude product is purified by reverse phase HPLC to is afford the title compound.

### 5-Guanidino-2-(S)-[3-phenyl-2-(R)-(3-phenyl-propylamino)-propionylamino]-pentanoic acid (2-naphthalen-1-yl-ethyl)-amide

To solution of 5-nitroguanidino-2-(S)-[3-phenyl-2-(R)-(3-phenyl-propylamino)-propionylamino]-pentanoic acid (2-naphthalen-1-yl-ethyl)-amide (280 mg, 0.44 moles) in 50 ml of methanol is added acetic acid (5 ml) and 5% palladium on barium sulfate (280 mg). The resulting suspension is hydrogenated at atmospheric pressure for twenty-four hours, filtered through celite, and the solvents are removed under reduced pressure. The crude material is purified by reverse-phase HPLC to afford the title compound.

### EXAMPLE 58

### Synthesis of 5-Guanidino-2-(S)-(2-(S)-{[3-(4-hydroxy-phenyl)-propionyl]-methyl-amino}-3-phenyl-propionylamino)-penta acid [2-(1H-indol-3yl)-ethyl]-amide

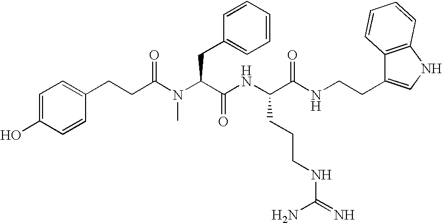

### 2-(S)-[2-(S)-(tert-Butoxycarbonyl-methyl-amino)-3-phenyl-propionylamino]-5-nitroguanidino-pentanoic acid methyl ester

To a solution of 2-(S)-(tert-butoxycarbonyl-methyl-amino)-3-phenyl-propionic acid (2.0 g 7.17 mmoles) and 2-(S)-amino-5-nitroguanidino-pentanoic acid methyl ester (1.83 g, 1.1 eq) is added 1-hydroxybenzotriazole (1.45 g, 1.5 eq), 1-[3-(dimethylaminopropyl]-3-ethylcarbodiimide hydrochloride (2.05 g, 1.5 eq), and triethylamine (3.0 ml, 3 eq). The resulting solution is stirred at room temperature for twenty-four hours, and then the solvents are removed under reduced pressure. The residue is partitioned between 10% sodium carbonate (150 ml) and methylene chloride (150 ml). The organics are dried over anhydrous magnesium sulfate, filtered and the solvents removed under reduced pressure. The crude material is purified by flash chromatography on silica gel (90:9:1 chloroform:methanol:ammonium hydroxide) to give the title compound.

### 5-Nitroguanidino-2-(S)-(2-(S)-methylamino-3-phenyl-propionylamino)-pentanoic acid methyl ester

To a solution of 2-(S)-[2-(S)-(tert-butoxycarbonyl-methyl-amino)-3-phenyl-propionylamino]-5-nitroguanidino-pentanoic acid methyl ester (3.3 g, 6.68 mmoles) in methylene chloride (200 ml) is added trifluoroacetic acid (100 ml). The resulting solution is stirred at room temperature for twenty-four hours, and then the solvents are removed under reduced pressure. The residue is partitioned between methylene chloride (150 ml) and 10% sodium carbonate (150 ml). The organics are dried over anhydrous magnewium sulfate, filtered, and the solvents removed under reduced pressure. The crude material is purified by flash chromatography on silica gel (90:9:1 chloroform:methanol:ammonium hydroxide) to afford the title compound.

### 5-Nitroguanidino-2-(S)-(2-(S)-{[3-(4-hydroxyphenyl)propionyl]-methyl-amino-3-phenyl-propionylamino)-pentanoic acid methyl ester

To a solution of 5-Guanidino-2-(S)-(2-(S)-methylamino-3-phenyl-propionylamino0-pentanoic acid methyl ester (1.0 g, 2.54 mmoles) and 3-(4-hydroxyphenyl)-propanoic acid (506 mg, 1.2 eq) is added 1-hydroxybenzotriazole (513 mg, 1.5 eq), 1-[3-(dimethylaminopropyl]-3-ethylcarbodiimide hydrochloride (727 mg, 1.5 eq) and triethylamine (1.0 ml, 3 eq). The resulting solution is stirred at room temperature for twenty-four hours, and then the solvents are removed under reduced pressure. The residue is partitioned between 10% sodium carbonate (100 ml) and methylene chloride (100 ml). The organics are dried over anhydrous magnesium sulfate, filtered, and the solvents removed under reduced pressure. The crude material is purified by flash chromatography on silica gel (90:9:1 chloroform:methanol:ammonium hydroxide) to afford the title compound.

### 5-Nitroguanidino-2-(S)-(2-(S)-{[3-(4-hydroxyphenyl)propionyl]-methyl-amino}-3-phenyl-propionylamino)-pentanoic acid

To a solution of 5-nitroguanidino-2-(S)-(2-(S)-{[3-(4-hydroxyphenyl)propionyl]-methyl-amino}-3-phenyl-propionylamino)-pentanoic acid methyl ester (534 mg, 0.98 mmoles) in tetrahydrofuran (50 ml) is added lithium hydroxide monohydrate (49 mg, 1.1 eq), and water (3 ml). The resulting solution is stirred at room temperature for twenty-four hours and then acidified with trifluoroacetic acid (1 eq). The solvents are removed under reduced pressure and the crude material partitioned between ethyl acetate (75 ml) and water (75 ml). The organics are dried over anhydrous magnewium sulfate, filtered, and the solvents removed under reduced pressure to afford the title compound.

### 5-Nitroguanidino-2-(S)-(2-(S)-{[3-(4-hydroxy-phenyl)-propionyl]-methyl-amino}-3-phenyl-propionylamino)-penta acid [2-(1H-indol-3yl)-ethyl]-amide

To a solution of 5-nitroguanidino-2-(S)-(2-(S)-{[3-(4-hydroxyphenyl)propionyl]-methyl-amino}-3-phenyl-propionylamino)-pentanoic acid (460 mg, 0.87 mmoles) and [2-(1H-indol-3yl)-ethyl]-amine (168 mg, 1.2 eq) is added 1-hydroxybenzotriazole (176 mg, 1.5 eq), 1-[3-(dimethylaminopropyl]-3-ethylcarbodiimide hydrochloride (250 mg, 1.5 eq) and triethylamine (0.36 ml, 3 eq). The resulting solution is stirred at room temperature for twenty-four hours, and then the solvents are removed under reduced pressure. The residue is partitioned between ethyl acetate (75 ml) and water (75 ml), the organics dried over magnesium sulfate, filteed and the solvents removed under reduced pressure. The crude product is purified be reverse-phase HPLC to afford the title compound.

### 5-Guanidino-2-(S)-(2-(S)-{[3-(4-hydroxy-phenyl)-propionyl]-methyl-amino}-3-phenyl-propionylamino)-penta acid [2-(1H-indol-3yl)-ethyl-amide

To solution of 5-nitroguanidino-2-(S)-(2-(S)-{[3-(4-hydroxy-phenyl)-propionyl]-methyl-amino}-3-phenyl-propionylamino)-penta acid [2-(1H-indol-3yl)-ethyl]-amide (250 mg, 0.37 moles) in 50 ml of methanol is added acetic acid (5 ml) and 5% palladium on barium sulfate (250 mg). The resulting suspension is hydrogenated at atmospheric pressure for twenty-four hours, filtered through celite, and the solvents removed under reduced pressure. The crude material is purified by reverse phase HPLC to afford the title compound.

### EXAMPLE 59

### Synthesis of 5-Guanidino-2-(S)-[3-phenyl-2-(R)-(3-phenyl-propylamino)-propionylamino]-pentanoic acid benzyl amide

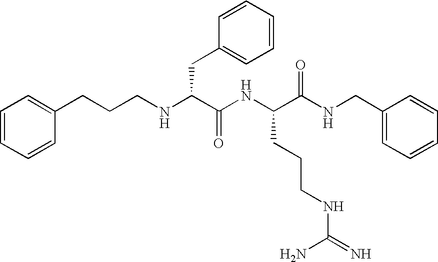

### [1-(S)-(1-Benzylcarbamoyl-4-nitroguanidino-butylcarbamoyl)-2-(R)-phenyl-ethyl]-carbamic acid tert-butyl ester

To a solution of 2-(S)-(2-(R)-tert-butoxycarbonylamino-3-phenyl-propioloylamino)-5-nitroguanidino-pentanoic acid (500 mg, 1.29 mmoles) benzylamine (0.155 ml, 1.1 eq) in DMF (50 ml) is added 1-hydroxybenzotriazole (261 mg, 1.5 eq), 1-[3-(dimethylaminopropyl]-3-ethylcarbodiimide hydrochloride (371 mg, 1.5 eq) and triethylamine (0.53 ml, 3 eq). The resulting solution is stirred at room temperature for twenty-four hours, and then the solvents are removed under reduced pressure. The crude material is purified by flash chromatography on silica gel (90:9:1 chloroform:methanol:ammonium hydroxide) to afford 526 mg the title compound.

### 2-(S)-(2-(R)-Amino-3-phenyl-propionylamino)-5-nitroguanidino-pentanoic acid benzylamide

To a solution of [1-(S)-(1-benzylcarbamoyl-4-nitroguanidino-butylcarbamoyl)-2-(R)-phenyl-ethyl]-carbamic acid tert-butyl ester (512 mg, 1.12 mmoles) in methylene chloride (50 ml) is added trifluororacetic acid (25 ml). The resulting solution is stirred at room temperature for twenty-four hours, and the solvents are then removed under reduced pressure. The crude product is purified by reverse-phase HPLC to afford the title compound.

### 5-Nitroguanidino-2-(S)-[3-phenyl-2-(R)-(3-phenyl-propylamino)-propionylamino]-pentanoic acid benzyl amide

To suspension of 2-(S)-(2-(R)-amino-3-phenyl-propionylamino)-5-nitroguanidino-pentanoic acid benzylamide (370 mg, 0.65 mmoles), 3-phenyl-propionaldehyde (0.077 ml, 0.9 eq) and molecular seives (370 mg, 4 A powdered) is added triethylamine (0.177 ml, 2 eq). The resulting suspension is stirred at room temperature for twenty-four hours, and then the pH is adjusted to 5 with acetic acid. Sodium cyanoborohydride (0.70 ml, 1.0M solution in tetrahydorofuran, 1 eq) is then slowly added (0.2 ml/hour) with a syringe pump. After twenty-four hours the suspension is filtered through celite and the solvents removed under reduced pressure. The crude product is purified by reverse-phase HPLC to afford the title compound.

### 5-Guanidino-2-(S)-[3-phenyl-2-(R)-(3-phenyl-propylamino)-propionylamino]-pentanoic acid benzyl amide

To a solution of 5-nitroguanidino-2-(S)-[3-phenyl-2-(R)-(3-phenyl-propylamino)-propionylamino]-pentanoic acid benzyl amide (80 mg, 0.14 mmoles) in methanol (50 ml) is added acetic acid (5 ml) and 5% palladium on abriLim sulfate (80 mg). The resulting suspension is hydrogenated at atmospheric pressure for twenty-four hours, filtered through celite and the solvents removed under reduced pressure. The crude product is purified by reverse-phase HPLC lo to afford the title compound.

### EXAMPLE 60

### Synthesis of 2-(S)-(2-(R)-[2-(S)-Acetylamino-3-(4-hydroxy-phenyl)-propionylamino]-3-phenyl-propionylamino}-5-nitroguanidino-pentanoic acid [1-carbamoyl-2-(1H-indol-3-yl)-ethyl]-amide

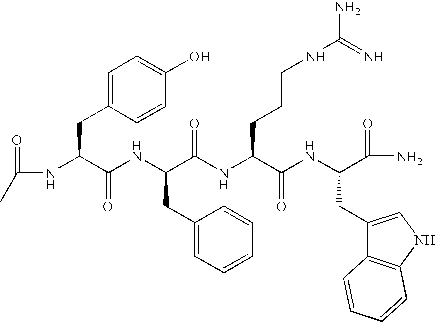

### (1-(S)-{1-[1-Carbamoyl-2-(1H-indol-3-yl)-ethylcarbamoyl]-4-nitroguanidino-butylcarbamoyl 1-2-(R)-phenyl-ethyl)-carbamic acid tert-butyl ester

To a solution of 2-(S)-(2-(R)-tert-butoxycarbonylamino-3-phenyl-propionylamino)-5-nitroguanidino-pentanoic acid (800 mg, 1.7 mmoles), and 2-(S)-amino-3-(1H-indol-3-yl)-propionamide (500 mg, 1.2 eq) in DMF (50 ml) is added 1-hydroxybenzotriazole (350 mg, 1.5 eq), 1-[3-(dimethylaminopropyl]-3-ethylcarbodiimide hydrochloride (491 mg, 1.5 eq) and triethylamine (1.17 ml, 5 eq). The resulting solution is stirred at room temperature for twenty-four hours and then the solvents are removed under reduced pressure. The residue is partitioned between ethyl acetate (75 ml) and 10% sodium carbonate (75 ml). The organics are dried over anhydrous magnesiumn sulfate, filtered and the solvents removed under reduced pressure. The crude product is purified by flash chormatography on silica gel (90:9:1 chloroform:methanol:ammonium hydroxide) to afford the title compound.

### 2-(S)-(2-(R)-Amino-3-phenyl-propionylamino)-5-nitroguanidino-pentanoic acid [1 -carbamoyl-2-(1H-indol-3-yl)-ethyl]-amide

To a solution of (1-(S)-{1-[1-carbamoyl-2-(1H-indol-3-yl)-ethylcarbamoyl]-4-nitroguanidino-butylcarbamoyl}-2-(R)-phenyl-ethyl)-carbamic acid tert-butyl ester (1.16 g, 1.78 mmoles) in methylene chloride (100 ml) is added trifluoroacetic acid (50 ml). The resulting solution is stirred at room temperature for twenty-four hours, and then the solvents are removed under reduced pressure. The crude material is purified by reverse-phase HPLC to afford the title compound.

### 2-(S)-2-(R)-[2-(S)-Acetylamino-3-(4-hydroxy-phenyl)-propionylamino]-3-phenyl-propionylamino-5-nitroguanidino-pentanoic acid [1-carbamoyl-2-(1H-indol-3-yl)-ethyl]-amide

To a solution of 2-(S)-(2-(R)-amino-3-phenyl-propionylamino)-5-nitroguanidino-pentanoic acid [1-carbamoyl-2-(1H-indol-3-yl)-ethyl]-amide (250 mg, 0.378 mmoles) and 2-(S)-acetylamino-3-(4-hydroxy-phenyl)-propionic acid (100 mg, 1.2 eq) in DMF (50 ml) is added 1-hydroxybenzotriazole (76 mg, 1.5 eq), 1-[3-(dimethylaminopropyl]-3-ethylcarbodiimide hydrochloride (107 mg, 1.5 eq) and triethylamine (0.20 ml, 4 eq). The resulting solution is stirred at room temperature for twenty-four hours, and then the solvents are removed under reduced pressure. The crude material is purified by reverse-phase HPLC to afford the title compound.

### 2-(S)-2-(R)-[2-(S)-Acetylamino-3-(4-hydroxy-phenyl)-propionylamino]-3-phenyl-propionylamino)-5-nitroguanidino-pentanoic acid [1-carbamoyl-2-(1H-indol-3-yl)-ethyl]-amide

To a solution of 2-(S)-{2-(R)-[2-(S)-acetylamino-3-(4-hydroxy-phenyl)-propionylamino]-3-phenyl-propionylamino}-5-nitroguanidino-pentanoic acid [1-carbamoyl-2-(1H-indol-3-yl)-ethyl]-amide (100 mg, 0.13 mmoles) in methanol (50 ml) is added acetic acid (5 ml) and 5% palladium on abrium sulfate (70 mg). The resulting suspension is hydrogenated at atmospheric pressure for twenty-four hours, filtered through celite and the solvents removed under reduced pressure. The crude product is purified by reverse-phase HPLC to afford the title compound.

### EXAMPLE 61

### Synthesis of 2-(2-(R)-Amino-3-phenyl-propionylamino)-5-(1-trityl-1H-imidazol-4-yl)-pentanoic acid [2-(S)-(1H-indol-3-yl)-1-methylcarbamoyl-ethyl]-amide

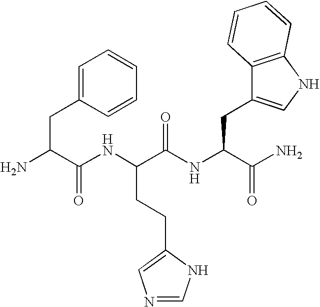

### 2-Amino-5-(3H-imidazol-4-yl)-pentanoic acid methyl ester

To a suspension of 2-tert-butoxycarbonylamino-5-(3H-imidazol-4-yl)-pentanoic acid (2.0 g, 11.8 mmoles) in methanol (60 ml) is added anhydrous hydrogen chloride until the solution is saturated. The solution is then heated to reflux for twenty-four hours and then cooled to room temperature. The solvents are removed under reduced pressure to afford the title compound.

### 2-(S)-(2-(R)-tert-Butoxycarbonylamino-3-phenyl-propionylamino)-5-(3H-imidazol-4-yl)-pentanoic acid methyl ester

To a solution of 2-(R)-(2-tert-butoxycarbonylamino-3-phenyl-propionic acid (500 mg, 1.88 mmoles) and 2-amino-5-(3H-imidazol-4-yl)-pentanoic acid methyl ester (500 mg, 1.1 eq ) is added hydroxybenzotriazole (381 mg, 1.5 eq), 1-[3-(dimethylaminopropyl]-3-ethylcarbodiimide hydrochloride (540 mg, 1.5 eq) and triethylamine (1.28 ml, 5 eq). The resulting solution is stirred at room temperature for twenty-four hours and then the solvents are removed under reduced pressure. The residue is partitioned between ethyl acetate (75 ml) and 10% sodium carbonate (75 ml). The organics are dried over anhydrous magnesium sulfate, filtered and the solvents removed under reduced pressure. The crude product is purified by flash chromatography on silica gel (90:9:1 chloroform:methanol:ammonium hydroxide) to afford the title compound.

### 2-(S)-2-(R)-tert-Butoxycarbonylamino-3-phenyl-propionylamino)5-(1-trityl-1H-imidazol-4-yl)-pentanoic acid methyl ester

To a solution of 2-(S)-(2-(R)-tert-butoxycarbonylamino-3-phenyl-propionylamino)-5-(3H-imidazol-4-yl)-pentanoic acid methyl ester (300 mg, 0.72 mmoles) in tetrahydrofuran (50 ml) is added triphenylmethylchloride (220 mg, 1.1 eq) and triethylamine (0.2 ml, 2 eq). The resulting solution is stirred at room temperature for twenty-four hours and then the solvents are removed under reduced pressure. The crude product is purified by flash chromatography on silica gel (5% methanol/chloroform) to afford the title compound.

### 2-(S )-(2-(R)-tert-Butoxycarbonylamino-3-phenyl-propionylamino)-5-(1-trityl-1H-imidazol-4-yl)-pentanoic acid

To a solution of 2-(S)-(2-(R)-tert-butoxycarbonylamino-3-phenyl-propionylamino)-5-(1-trityl-1H-imidazol-4-yl)-pentanoic acid methyl ester (300 mg, 0.45 mmoles) in tetrahydrofuran (30 ml) is added lithium hydroxide monohydrate (32 mg, 1.2 eq) and water (3 ml) the resulting solution is stirred at room temperature for twenty-four hours, and then the solvents are removed under reduced pressure. The crude material is used directly in the next step.

### {1-(R)-[1-[2-(S)-(1H-Indol-3-yl)-1-methylcarbamoyl-ethylcarbamoyl]-4-(1-trityl-1H-imidazol-4-yl)-butylcarbamoyl]-2-phenyl-ethyl}-carbamic acid tert-butyl ester

To a solution of 2-(S)-(2-(R)-tert-butoxycarbonylamino-3-phenyl-propionylamino)-5-(1-trityl-1H-imidazol-4-yl)-pentanoic acid (293 mg, 0.45 mmoles) and 2-(S)-amino-3-(1H-indol-3-yl)-propionamide (117 mg, 1.3 eq) is added PyBOP (301 mg, 1.3 eq) and triethylamine (0.18 ml, 3 eq). The resulting solution is stirred at room temperature for twenty-four hours, and then the solvents are removed under reduced pressure. The residue is partitioned between ethy acetate (75 ml) and 19% sodium carbonate (75 ml). The organics are dried over anhydrous magnesium sulfate, filtered, and the solvents removed under reduced pressure. The crude product is purified by flash chromatography on silica gel (5% methanol/chloroform) to afford the title compound.

### 2-(2-(R)-Amino-3-phenyl-propionylamino)-5-(1-trityl-1H-imidazol-4-yl)-pentanoic acid [2-(S)-(1H-indol-3-yl)-1-methylcarbamoyl-ethyl]-amide

To a solution of {1-(R)-[1-[2-(S)-(1H-Indol-3-yl)-1-methylcarbamoyl-ethylcarbamoyl]-4-(1-trityl-1H-imidazol-4-yl)-butylcarbamoyl]-2-phenyl-ethyl}-carbamic acid tert-butyl ester (468 mg, 0.55 mmoles) in methylene chloride (32 ml) is added trifluoroacetic acid (16 ml). Triethylsilane is then added dropwise until the bright yellow color disappeared. The resulting solution is stirred at room temperature for twenty-four hours and then the solvents are removed under reduced pressure. The crude material is purified by reverse phase HPLC to afford the title compound.

### EXAMPLE 62

### Synthesis of 2-(R)-[2-(S)-(2-Benzyl-6-phenyl-hexanoylamino)-5-guanidino-pentanoylamino]-3-naphthalen-2-yl-propionic acid methyl ester

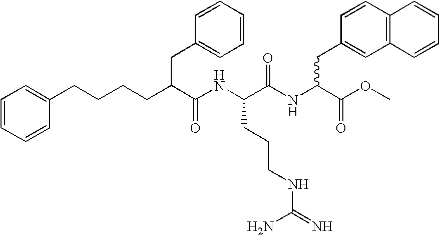

### 6-Phenyl-hexanoic acid methyl ester

A solution of 6-phenyl-hexanoic acid (1.9 g, 9.89 mmoles) in methanol (50 ml) is saturated with anhydrous hydrogen chloride and then heated to reflux for twenty-four hours. After cooling to room temperature, the solvents are removed under reduced pressure and the residue partitioned between chloroform and 10% sodium carbonate. The organics are dried over anhydrous magnesium sulfate, filtered and the solvent removed under reduced pressure to afford the title compound.

### 2-Benzyl-6-phenyl-hexanoic acid methyl ester

To a cooled (−78° C.) solution of 6-phenyl-hexanoic acid methyl ester (2.8 g, 13.5 mmoles) in anhydrous tetrahydrofuran (50 ml) is slowly added a 2.0M solution of lithium diisopropylamide hexane/tetrahydrofuran (7.5 ml, 1.1 eq). The resulting solution is stirred at −78° C. for fifty minutes and then benzyl bromide (1.92 ml, 1.2 eq) is slowly added. The resulting solution is warmed to room temperature overnight and then the solvents are removed under reduced pressure. The residue is then partitioned between ethyl acetate and water. The organics are dried over anhydrous magnesium sulfate filtered and the solvents removed under reduced pressure. The crude product is purified by reverse-phase HPLC to afford the title compound.

### 2-Benzyl-6-phenyl-hexanoic acid

To a solution of 2-benzyl-6-phenyl-hexanoic acid methyl ester (2.17 g, 7.33 mmoles), in tetrahydrofuran (100 ml) is added lithium hydroxide monohydrate (880 mg, 2 eq) and water (15 ml). The resulting solution is heated to reflux for forty-eight hours and then cooled to room temperature. The solvents are removed under reduced pressure and the residue partitioned between ethyl acetate and 1M citric acid. The organics are dried over anhydrous magnesium sulfate, filtered and the solvents removed under reduced pressure. The crude product is purified by reverse phase HPLC to afford the title compound.

### 2-(S)-(2-Benzyl-6-phenyl-hexanoylamino)-5-nitroguanidino-pentanoic acid methyl ester

To a solution of 2-benzyl-6-phenyl-hexanoic acid methyl ester (513 mg, 1.82 mmoles) and 2-(S)-amino-5-nitroguanidino-pentanoic acid methyl ester (480 mg, 1.1 eq) in DMF (50 ml) is added 1-hydroxybenzotriazole (319 mg, 1.3 eq), 1-[3-(dimethylaminopropyl]-3-ethylcarbodiimide hydrochloride (451 mg, 1.3 eq) and triethylamine (0.74 ml, 3 eq). The resulting solution is stirred at room temperature for twenty-four hours, and then the solvents are removed under reduced pressure. The crude material is purified by reverse-phase HPLC to afford the title compound.

### 2-(S)-(2-Benzyl-6-phenyl-hexanoylamino)-5-nitroguanidino-pentanoic acid

To a solution of 2-(S)-(2-benzyl-6-phenyl-hexanoylamino)-5-nitroguanidino-pentanoic acid methyl ester (700 mg, 1.5 mmoles) in tetrahydrofuran (40 ml) is added lithium hydroxide monohydrate (180 mg, 2 eq), and water (3 ml). The resulting solution is stirred at room temperature for twenty-four hours and then the solvents are removed under reduced pressure. The crude product is purified by reverse-phase HPLC to afford the title compound.

### 2-Amino-3-naphthalen-2-yl-propionic acid methyl ester

To a suspension of 2-amino-3-naphthalen-2-yl-propionic acid (300 mg, 1.39 mmoles) in methanol (40 ml) is added anhydrous hydrogen chloride until the solution is saturated. The resulting solution is heated to reflux for two hours and then cooled to room temperature, and the solvents removed under reduced pressure to afford the title compound.

### 2-(R)-[2-(S)-(2-Benzyl-6-phenyl-hexanoylamino)-5-nitroguanidino-pentanoylamino]-3-naphthalen-2-yl-propionic acid methyl ester

To a solution of 2-(S)-(2-benzyl-6-phenyl-hexanoylamino)-5-nitroguanidino-pentanoic acid (100 mg, 0.22 mmoles) and 2-amino-3-naphthalen-2-yl-propionic acid methyl ester (65 mg, 1.1 eq) in DMF (30 ml) is added 1-hydroxybenzotriazole (44 mg, 1. eq), 1-[3-(dimethylaminopropyl]-3-ethylcarbodiimide hydrochloride (63 mg, 1.5 eq) and triethyl amine (0.09 ml, 3 eq). The resulting solution is stirred at room temperature for twenty-four hours and then the solvents are removed under reduced pressure. The crude material is purified by reverse-phase HPLC to afford the title compound.

### 2-(R)-[2-(S)-(2-Benzyl-6-phenyl-hexanoylamino)-5-guanidino-pentanoylamino]-3-naphthalen-2-yl-propionic acid methyl ester

To a solution of 2-(R)-[2-(S)-(2-benzyl-6-phenyl-hexanoylamino)-5-nitroguanidino-pentanoylamino]-3-naphthalen-2-yl-propionic acid methyl ester (80 mg, 0.1 mmoles) in methanol (30 ml) is added acetic acid (3 ml) and 5% palladium on barium sulfate (75 mg). The resulting suspension is hydrogenated at atmospheric pressure for twenty-four hours, filtered through celite, and the solvents removed under reduced pressure. The crude product is purified by reverse-phase HPLC to afford the title compound as a mixture of four diastereomers.

### EXAMPLE 63

### Synthesis of 5-(1H-Imidazol-4-yl)-2-[3-phenyl-2-(R)-(3-phenyl-propionylamino)-propionylamino]-pentanoic acid (1-(S)-methylcarbamoyl-2-naphthalen-2-yl-ethyl)-amide

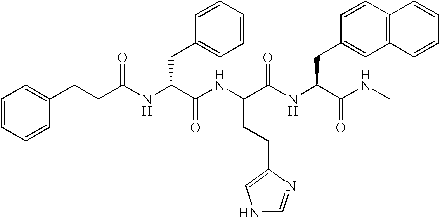

### 3-Phenyl-2-(R)-(3-phenyl-propionylamino)-propionic acid methyl ester

To a solution of 3-phenyl-propionic acid (1.0 g, 6.66 mmoles) and 2-(R)-amino-3-phenyl-propionic acid methyl ester (1.19 g, 1 eq) in DMF (60 ml) is added 1-hydroxybenzatriazole (1.35 g, 1.5 eq), 1-[3-(dimethylaminopropyl]-3-ethylcarbodiimide hydrochloride (1.90 g, 1.5 eq) and triethylamine (2.7 ml, 3 eq). The resulting solution is stirred at room temperature for twenty-four hours, and then the solvents are removed under reduced pressure. The residue is partitioned between water and ethyl acetate, dried over anhydrous magnesium sulfate, filtered and the solvents removed under reduced pressure to afford the title compound

### 3-Phenyl-2-(R)-(3-phenyl-propionylamino)-propionic acid

To a solution of 3-phenyl-2-(R)-(3-phenyl-propionylamino)-propionic acid methyl ester (1.5 g, 4.82 mmoles) in tetrahydrofuran (70 ml) is added lithium hydroxide monohydrate (434 mg, 1.5 eq) and water (5 ml). The resulting solution is stirred at room temperature for twenty-four hours, and then the solvents are removed under reduced pressure. The crude material is purified by reverse-phase HPLC to afford the title compound.

### 5-(3H-Imidazol-4-yl)-2-[3-phenyl-2-(R)-(3-phenyl-propionylamino)-propionylamino]-pentanoic acid methyl ester

To a solution of 3-phenyl-2-(R)-(3-phenyl-propionylamino)-propionic acid (376 mg, 1.26 mmoles) and 2-amino-5-(3H-imidazol-4-yl)-pentanoic acid methyl ester (355 mg, 1.1 eq) in DMF (50 ml ) is added 1-hydroxybenzotriazole (256 mg, 1.5 eq), 1-[3-(dimethylaminopropyl]-3-ethylcarbodiimide hydrochloride (362 mg, 1.5 eq) and triethylamine (0.863 ml, 5 eq). The resulting solution is stirred at room temperature for twenty-four hours and then the solvents are removed under reduced pressure. The crude product is purified by reverse-phase HPLC to afford the title compound.

### 2-[3-Phenyl-2-(R)-(3-phenyl-propionylamino)-propionylamino]-5-(1-trityl-1H-imidazol-4-yl)-pentanoic acid methyl ester

To a solution of 5-(3H-imidazol-4-yl)-2-[3-phenyl-2-(R)-(3-phenyl-propionylamino)-propionylamino]-pentanoic acid methyl ester (473 mg, 1.02 mmoles) in tetrahydrofuran (50 ml) is added triphenylmethylchloride (341 mg, 1.2 eq), and triethylamine (0.42 ml, 3 eq). The resulting solution is stirred at room temperature for twenty-four hours and then the solvents are removed under reduced pressure. The crude product is purified by flash chromatography on silica gel (5% methanol/chloroform) to afford the title compound.

### 2-[3-Phenyl-2-(R)-(3-phenyl-propionylamino)-propionylamino]-5-(1-trityl-1H-imidazol-4-yl)-pentanoic acid

To a solution of 2-[3-phenyl-2-(R)-(3-phenyl-propionylamino)-propionylamino]-5-(1-trityl-1H-imidazol-4-yl)-pentanoic acid methyl ester (380 mg, 0.54 mmoles) in tetrahydrofuran (80 ml) is added lithium hydroxide monohydrate (48 mg, 1.5 eq) and water (10 ml). The resulting solution is stirred at room temperature for twenty-four hours, and then the solvents are removed under reduced pressure. The crude material is used directly in the next step.

### (1-(S)-Methylcarbamoyl-2-naphthalen-2-yl-ethyl)-carbamic acid tert-butyl ester

To a solution of 2-(S)-tert-butoxycarbonylamino-3-naphthalen-2-yl-propionic acid (1.5 g, 4.76 mmoles) in DMF (50 ml) is added methyl amine (3.0 ml of a 2.0M solution in tetrahydrofuran), PyBOP (3.7 g, 1.5 eq), and triethylamine (1.95 ml, 3 eq). The resulting solution is stirred at room temperature for twenty-four hours, and then the solvents are removed under reduced pressure. The residue is partitioned between ethyl acetate and 10% sodium carbonate, the organics are dried over anhydrous magnesium sulfate, filtered and the solvents removed under reduced pressure. The crude product is purified by flash chromatography on silica gel (5% methanol/chloroform) to afford the title compound.

### 2-(S)-Amino-N-methyl-3-naphthalen-2-yl-propionamide

To a solution of (1-(S)-methylcarbamoyl-2-naphthalen-2-yl-ethyl)-carbamic acid tert-butyl ester (1.3 g, 3.96 mmoles) in methylene chloride (100 ml) is added trifluoroacetic acid (50 ml). The resulting solution is stirred at room temperature for twenty-four hours, and then the solvents are removed under reduced pressure. The crude material is purified by reverse-phase HPLC to afford of the title compound.

### 2-(S)-[3-Phenyl-2-(R)-(3-phenyl-propionylamino)-propionylamino]-5-(1-trityl-1H-imidazol-4-yl)-pentanoic acid (1-(S)-methylcarbamoyl-2-naphthlen-2-yl-ethyl)-amide

To a solution of 2-[3-phenyl-2-(R)-(3-phenyl-propionylamino)-propionylamino]-5-(1-trityl-1H-imidazol-4-yl)-pentanoic acid (383 mg, 0.53 mmoles) in DMF (50 ml) is added 2-(S)-amino-N-methyl-3-naphthalen-2-yl-propionamide (147 mg, 0.8 eq), PyBOP (420 mg, 1.5 eq) and triethyl amine (0.220 ml, 3 eq). The resulting solution is stirred at room temperature for forty-eight hours, and then the solvents are removed under reduced pressure. The residue is partitioned between ethyl acetate and 10% sodium carbonate, the organics are dried over anhydrous magnesium sulfate, filtered and the solvents removed under reduced pressure. The crude product is purified by flash chromatography on silica gel (5% methanol/chloroform) to afford the title compound.

### 5-(1H-Imidazol-4-yl)-2-[3-phenyl-2-(R)-(3-phenyl-propionylamino)-propionylamino]-pentanoic acid (1-(S)-methylcarbamoyl-2-naphthalen-2-yl-ethyl)-amide

To a solution of trifluoroacetic acid (20 ml) in methylene chloride (40 ml) is added 2-(S)-[3-phenyl-2-(R)-(3-phenyl-propionylamino)-propionylamino]-5-(1-trityl-1H-imidazol-4-yl)-pentanoic acid (1-methylcarbamoyl-2-naphthlen-2-yl-ethyl)-amide (441 mg, 0.489 mmoles). Triethylsilane is then added dropwise until the bright yellow color just disappeared. The resulting solution is stirred at room temperature for twenty-four hours and then the solvents are removed under reduced pressure. The crude product is purified by flash chromatography on silica gel (90:9:1 chloroform:methanol:ammonium hydroxide) to afford the title compound as a mixture of diastereomers. The diastereomers are separated by reverse-phase HPLC to afford an earlier eluting diastereomer and a later eluting diastereomer.

### EXAMPLE 64

### Synthesis of 2-(S)-{2-(R)-[2-(S)-Acetylamino-3-(4-hydroxy-phenyl)-propionylamino]-3-phenyl-propioloylamino}-5-guanidino-pentanoic acid (1-(S)-methylcarbamoyl-2-naphthalen-2-yl-ethyl)-amide

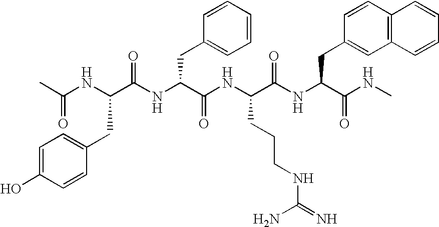

### {1-(R)-[4-Nitroguanidino-1-(S)-(1-(S)-methylcarbamoyl-2-naphthalen-2-yl-ethylcarbamoyl)-butylcarbamoyl]-2-phenyl-ethyl}-carbamic acid tert-butyl ester

To a solution of 2-(S)-(2-(R)-tert-butoxycarbonylamino-3-phenyl-propionylamino)-5-nitroguanidino-pentanoic acid (500 mg, 1.07 mmoles) and 2-(S)-amino-N-methyl-3-naphthalen-2-yl-propionamide (440 mg, 1.2 eq), is added PyBOP (836 mg, 1.5 eq) and triethylamine (0.58 ml, 4 eq). The resulting solution is stirred at room temperature overnight and then the solvents are removed under reduced pressure. The residue is partitioned between ethyl acetate and 10% sodium carbonate, dried over anhydrous magnesium sulfate, filtered and the solvents removed under reduced pressure. The crude product is purified by flash chromatography on silica gel (90:9:1 chloroform:methanol:ammonium hydroxide) to afford the title compound.

### 2-(S)-(2-(R)-Amino-3-phenyl-propionylamino)-5-nitroguanidino-pentanoic acid (1-(S)-methylcarbamoyl-2-naphthalen-2-yl-ethyl)-amide

To a solution of {1-(R)-[4-nitroguanidino-1-(S)-(1-(S)-methylcarbamoyl-2-naphthalen-2-yl-ethylcarbamoyl)-butylcarbamoyl]-2-phenyl-ethyl }-carbamic acid tert-butyl ester (500 mg, 0.74 mmoles) in methylene chloride (60 ml) is added trifluoroacetic acid (30 ml). The resulting solution is stirred at room temperature for twenty-four hours, and then the solvents are removed under reduced pressure. The crude product is purified by reverse-phase HPLC to afford the title compound.

### 2-(S)-{2-(R)-[2-(S)-Acetylamino-3-(4-hydroxy-phenyl)-propionylamino]-3-phenyl-propionylamino}-5-nitroguanidino-pentanoic acid (1-(S)-methylcarbamoyl-2-naphthalen-2-yl-ethyl)-amide

To a solution of 2-(S)-(2-(R)-amino-3-phenyl-propionylamino)-5-nitroguanidino-pentanoic acid (1-(S)-methylcarbamoyl-2-naphthalen-2-yl-ethyl)-amide (400 mg, 0.58 mmoles) in DMF (60 ml) is added 2-(S)-acetylamino-3-(4-hydroxy-phenyl)-propionic acid (155 mg, 1.2 eq), PyBOP (410 mg, 1.2 eq), and triethylamine (0.32 ml, 4 eq). The resulting solution is stirred at room temperature for twenty-four hours, and then the solvents are removed under reduced pressure. The residue is partitioned between ethyl acetate and 10% sodium carbonate, the organics are dried over anhydrous magnesium sulfate, filtered and the solvents removed under reduced pressure. The crude product is purified by flash chromatography on silica gel (90:9:1 chloroform:methanol:ammonium hydroxide) to afford the title compound.

### 2-(S)-{2-(R)-[2-(S)-Acetylamino-3-(4-hydroxy-phenyl)-propionylamino]-3-phenyl-propionylamino}-5-guanidino-pentanoic acid (1-(S)-methylcarbamoyl-2-naphthalen-2-yl-ethyl)-amide

To a solution of 2-(S)-{2-(R)-[2-(S)-acetylamino-3-(4-hydroxy-phenyl)-propionylamino]-3-phenyl-propionylamino)-5-nitroguanidino-pentanoic acid (1-(S)-methylcarbamoyl-2-naphthalen-2-yl-ethyl)-amide (330 mg, 0.42 mmoles) in methanol (50 ml) is added acetic acid (5 ml) and 5% palladium on barium sulfate (325 mg). The resulting suspension is hydrogenated at atmospheric pressure for twenty-four hours, and then filtered through celite. The solvents are removed reduced pressure and the crude product purified by reverse-phase HPLC to afford the title compound.

### EXAMPLE 65

### Synthesis of 5-Guanidino-2-(S)-{2-(R)-[3-(4-hydroxy-phenyl)-propionylamino]-3-phenyl-propionylamino}-pentanoic acid (1-(S)-methylcarbamoyl-2-naphthalen-2-yl-ethyl)-amide

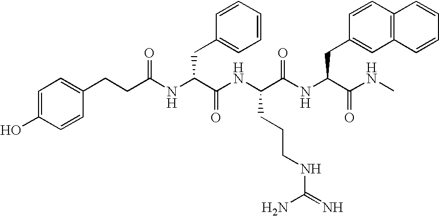

To a solution of 2-(S)-(2-(R)-amino-3-phenyl-propionylamino)-5-nitroguanidino-pentanoic acid (1-(S)-methylcarbamoyl-2-naphthalen-2-yl-ethyl)-amide (268 mg, 3.88 mmoles) and 3-(4-hydroxy-phenyl)-propionic acid (77 mg, 1.2 eq) is added 1-[3-(dimethylaminopropyl]-3-ethylcarbodiimide hydrochloride (111 mg, 1.5 eq), 1-hydroxybenzotriazole (78 mg, 1.5 eq) and triethylamine (0.21 ml, 4 eq). The resulting solution is stirred at room temperature for twenty-four hours and then the solvents are removed under reduced pressure. The crude material is purified by reverse-phase HPLC to afford the title compound.

### 5-Guanidino-2-(S)-{2-(R)-13-(4-hydroxy-phenyl)-propionylamino -3-phenyl-propionylamino}-pentanoic acid (1-(S)-methylcarbamoyl-2-naphthalen-2-yl-ethyl)-amide.

To a suspension of 5-nitroguanidino-2-(S)-{2-(R)-[3-(4-hydroxy-phenyl)-propionylamino]-3-phenyl-propionylamino}-pentanoic acid (1-(S)-methylcarbamoyl-2-naphthalen-2-yl-ethyl)-amide (141 mg, 0.19 mmoles), and 5% palladium on barium sulfate (100 mg) in methanol (45 ml) is added acetic acid (5 ml). The resulting suspension is hydrogenated at atmospheric pressure four twenty-four hours, filtered through celite, and the solvents are removed under reduced pressure. The crude product is purified by reverse-phase HPLC to afford the title compound.

### EXAMPLE 66

### Synthesis of 5-Guanidino-2-(S)-[3-phenyl-2-(R)-(2-phenyl-ethanesulfonylamino)-propionylamino]-pentanoic acid (1-(S)-methylcarbamoyl-2-naphthalen-2-yl-ethyl)-amide

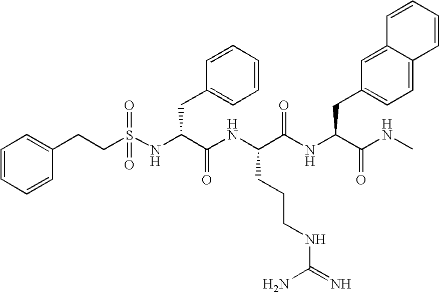

### 2-Phenyl-ethanesulfonyl chloride

To a biphasic solution of 2-phenyl-ethanethiol (10.0 g, 72.5 mmoles) in 100 ml of ice water is added acetic acid (20 ml). This solution is then saturated with chlorine gas for five minutes. The aqueous solution is then extracted with ethyl ether, dried over anhydrous magnesium sulfate, filtered and the solvents removed under reduced pressure to afford the title compound.

### 3-Phenyl-2-(R)-(2-phenyl-ethanesulfonylamino)-propionic acid methyl ester

To a solution of 2-amino-3-phenyl-propionic acid methyl ester (700 mg, 3.9 mmoles) in tetrahydrofuran (30 ml) is added dropwise 2-phenyl-ethanesulfonyl chloride (1.2 g, 5.88 mmoles). To this solution is added triethylamine (1.55 ml, 3 eq). The resulting solution is stirred at room temperature for twenty-four hours and then the solvents are removed under reduced pressure. The crude product is purified by reverse-phase HPLC to afford the title compound.

### 3-Phenyl-2-(R)-(2-phenyl-ethanesulfonylamino)-propionic acid

To a solution of 3-phenyl-2-(R)-(2-phenyl-ethanesulfonylamino)-propionic acid methyl ester (890 mg, 2.56 mmoles) in tetrahydrofuran (50 ml) is added lithium hydroxide monohydrate (260 mg, 1.5 eq) and water (5 ml). The resulting solution is stirred at room temperature for twenty-four hours and then the solvents are removed under reduced pressure. The crude product is purified by reverse-phase HPLC to afford the title compound.

### [4-Nitroguanidino-1-(R)-(1-(S)-methylcarbamoyl-2-naphthalen-2-yl-ethylcarbamoyl)-butyl]-carbamic acid tert-butyl ester

To a solution of 2-amino-N-methyl-3-naphthaolen-2-yl-propionamide (1.0 g, 2.92 mmoles) and 2-(R)-tert-butoxycarbonylamino-5-nitroguanidino-pentanoic acid (1.12 g, 1.2 eq) is added 1-[3-(dimethylaminopropyl]-3-ethylcarbodiimide hydrochloride (837 mg, 1.5 eq), 1-hydroxybenzotriazole (592 mg, 1.5 eq), and triethylamine (1.2 ml, 3 eq). The resulting suspension is stirred at room temperature for twenty-four hours and then the solvents are removed under reduced pressure. The residue is partitioned between ethyl acetate and 10% sodium carbonate, the organics are dried over anhydrous magnesium sulfate, filtered, and the solvents removed under reduced pressure. The crude product is purified by reverse-phase HPLC to afford the title compound.

### 2-(R)-Amino-5-guanidino-pentanoic acid (1-(S)-methylcarbamoyl-2-naphthalen-2-yl-ethyl)-amide

To a solution of [4-nitroguanidino-1-(R)-(1-(S)-methylcarbamoyl-2-naphthalen-2-yl-ethylcarbamoyl)-butyl]-carbamic acid tert-butyl ester (800 mg, 1.52 mmoles) in methylene chloride (40 ml) is added trifluoroacetic acid (20 ml). The resulting solution is stirred at room temperature for twenty-four hours and then the solvents are removed under reduced pressure. The crude product is purified by reverse-phase HPLC to afford the title compound as the trifluoroacetic acid salt.

### 5-Nitroguanidino-2-(S)-[3-phenyl-2-(R)-(2-phenyl-ethanesulfonylamino)-propionylamino]-pentanoic acid (1-(S)-methylcarbamoyl-2-naphthalen-2-yl-ethyl)-amide

To a solution of 3-phenyl-2-(R)-(2-phenyl-ethanesulfonylamino)-propionic acid (150 mg, 0.45 mmoles) and 2-(R)-amino-5-guanidino-pentanoic acid (1-(S)-methylcarbamoyl-2-naphthalen-2-yl-ethyl)-amide (270 mg, 1.1 eq) is added 1-[3-(dimethylaminopropyl]-3-ethylcarbodiimide hydrochloride (130 mg, 1.5 eq), 1-hydroxybenzotriazole (91 mg, 1.5 eq), and triethyl amine (0.25 ml, 4 eq). The resulting solution is stirred at room temperature for twenty-four hours and then the solvents are removed under reduced pressure. The crude product is purified by preparative HPLC to afford the title compound.

### 5-Guanidino-2-(S)-3-phenyl-2-(R)-(2-phenyl-ethanesulfonylamino)-propionylamino]-pentanoic acid (1-(S)-methylcarbamoyl-2-naphthalen-2-yl-ethyl)-amide

To a solution of 5-nitroguanidino-2-(S)-[3-phenyl-2-(R)-(2-phenyl-ethanesulfonylamino)-propionylamino]-pentanoic acid (1-(S)-methylcarbamoyl-2-naphthalen-2-yl-ethyl)-amide (230 mg, 0.31 mmoles) in methanol (45 ml) is added acetic acid (5 ml) and 5% palladium on barium sulfate (200 mg). The resulting suspension is hydrogenated at atmospheric pressure for twenty-four hours, and then filtered through celite. The solvents are removed under reduced pressure and the crude product purified by reverse-phase HPLC to afford the title compound as the trifluoroacetic acid salt.

### EXAMPLE 67

### Synthesis of Ac-(carba-Ff)-RW—NH₂ (11)

This compound is prepared according to the plans presented in Schemes IA&B:

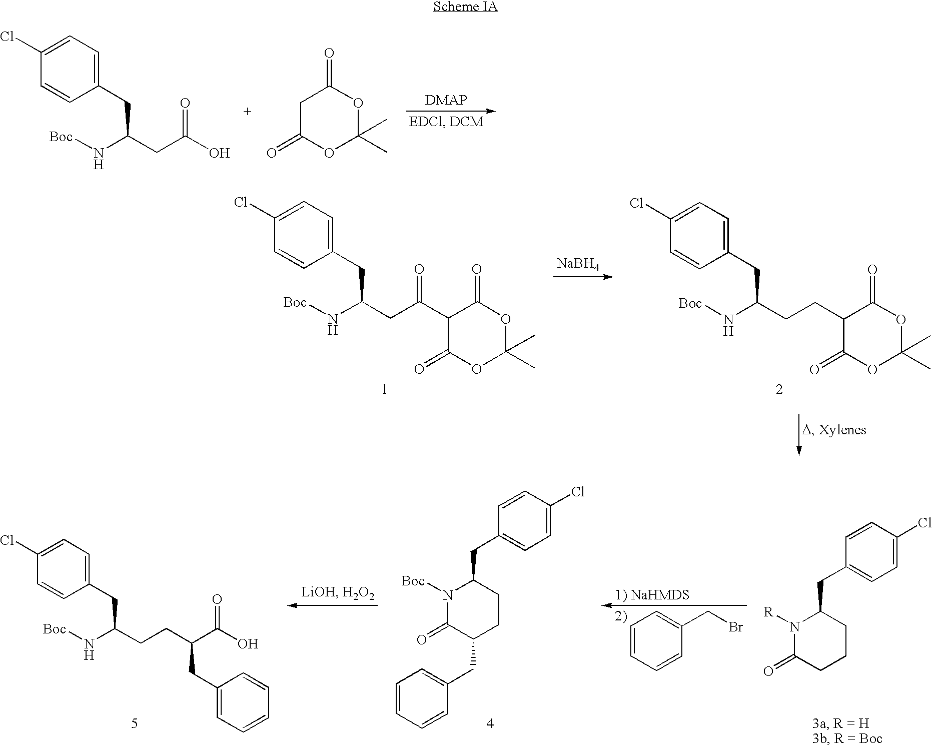

### [1-(4-Chloro-benzyl)3-(2,2-dimethyl-4,6-dioxo-[1,3]dioxan-5-yl)-3-oxo-propyl]-carbamic acid tert-butyl ester (1)

To a well-stirred mixture of Boc-(S)-3-amino-4-(4-chlorophenyl)-butyric acid (5.0 g, 16 mmol), 2,2-dimethyl-1,3-dioxane-4,6-dione (2.54 g, 17.6 mmol) and DMAP (24 mmol) in DCM (160 ml) at 0° C. is added EDCI (24 mmol) in one portion. The resulting mixture is stirred at 0° C. for 1 h, then at room temperature for 18 h. DCM (100 ml) is added and the mixture is washed with water (2×50 ml), 5% aqueous potassium hydrogen sulfate (3×50 ml), 5% aqueous sodium bicarbonate (1×50 ml), and brine (1×50 ml). The organic layer is dried over anhydrous magnesium sulfate and concentrated to yield 1.

### [1-(4-Chloro-benzyl)3-(2,2-dimethyl-4,6-dioxo-[1,3]dioxan-5-yl)-propyl]-carbamic acid tert-butyl ester (2)

Sodium borohydride (63.8 mmol, 4.0 eq.) is added portionwise over 1 h to a well-stirred solution of 1 15.96 mmol) in a mixture of DCM (180 ml) and acetic acid (10 ml, 175 mmol, 11.0 eq.) at 0° C. The resulting mixture is stirred at 0° C. for 1 h and then at room temperature for 64 h. The reaction mixture is diluted with DCM (150 ml) and washed with water (1×50 ml) and brine (2×50 ml). The organic layer is dried over anhydrous magnesium sulfate and concentrated by rotary evaporation to yield 2.

### 6-(4-Chloro-benzyl)-piperidin-2-one (3a) and 2-(4-Chloro-benzyl)-6-oxo-piperidine-1-carboxylic acid tert-butyl ester (3b)

A stirred mixture of 2 (15.32 mmol) and xylene (140 ml) is heated at reflux for 6 h. The solvent is removed by evaporation at 37° C. in vacuo to yield crude 3a. This material is combined with di-tert-butyl dicarbonate (5.0 eq.) and DMAP (0.3 eq.) in DCM (100 ml) and the mixture is stirred at room temperature for 40 h. The solvent is removed by rotary evaporation and the residue is purified by flash chromatography on silica gel using EtOAc-hexane (1:19, 500 ml and 1:9, 1300 ml) as an eluant to yield 3b.

### 3-Benzyl-6-(4-Chloro-benzyl)-2-oxo-piperidine-1-carboxylic acid tert-butyl ester (4)

Sodium bis(trimethylsilyl)amide (5.16 ml of a 1.0 M solution in THF, 1.0 eq.) is added dropwise to a well-stirred solution of 3b (1.67 g, 5.16 mmol) in a mixture of THF-DME (1:1, 100 ml) at −78° C. under argon and the resulting mixture is stirred for 0.5 h more at −78° C. To this stirred, cold mixture is added a solution of benzyl bromide (0.882 g, 5.16 mmol, 1.0 eq.) in THF (5 ml) and stirring is continued at −78° C. under argon for 2 h. The reaction mixture is quenched with a saturated aqueous solution of ammonium chloride (20 ml) and stirring is continued for 10 min. more. The mixture is then partitioned between DCM (80 ml) and water (40 ml) and the water phase is extracted with DCM (2×40 ml). The combined organic phase is washed with water (1×40 ml), dried over anhydrous magnesium sulfate, and concentrated to give crude 4, which is purified by flash column chromatography on silica gel using EtOAc-hexane (1:39, 1000 ml and 1: 19, 1300 ml) as an eluant to afford 4 (single diastereomer).

### 2-(R)-Benzyl-5-(R)-tert-butoxycarbonylamino-6-(4-chloro-phenyl)-hexanoic acid (‘Boc-(carba-4-Cl—Ff)-OH’5)

To a well-stirred solution of 4 (1.152 g, 2.783 mmol) in a mixture of THF-water (4:1) are added lithium hydroxide monohydrate (0.467 g, 11.132 mmol, 4 eq.) in one portion and 35% hydrogen peroxide (1.95 ml, 8.0 eq.) by dropwise addition. The resulting mixture is stirred at 0° C. for 1 h and then at room temperature for 16 h. The reaction mixture is acidified with aqueous hydrochloric acid (11.5 ml, 1N) at 0° C. and is extracted with DCM (4×40 ml). The organic layer is dried over anhydrous magnesium sulfate, concentrated, and the residue triturated to yield 5 (single diastereomer).

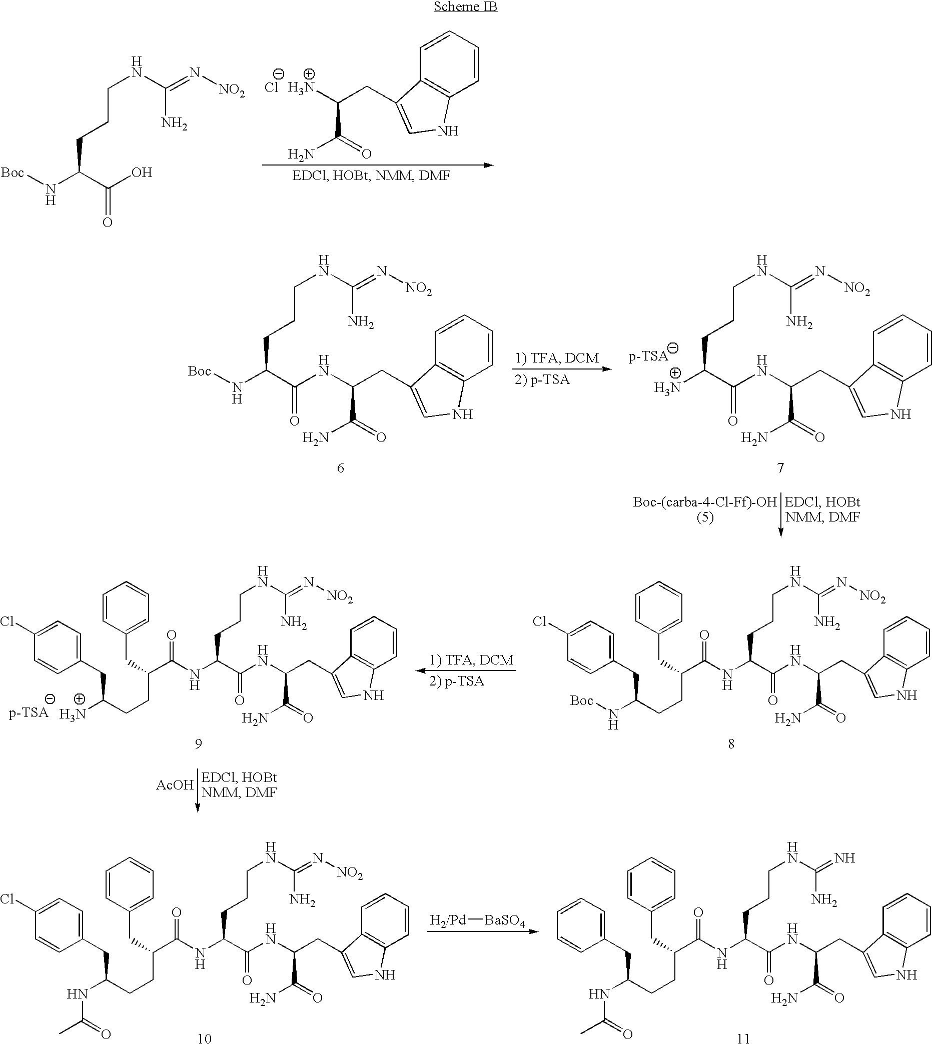

### Boc-R(NO₂)—W—NH₂(6)

HOBt (1.0 eq.) is added to a well-stirred solution of Boc-R(NO₂)—OH (0.639 g, 2.0 mmol) and H—w—NH₂.HCl (0.479 g, 2.0 mmol) in DMF (10 ml) at 0° C. followed by addition of EDCI (1.1 eq.) and NMM (0.484 ml, 2.2 eq.). The resulting mixture is stirred at 0° C. for 1 h and then at room temperature for 4-18 h. The reaction mixture is diluted with EtOAc (100 ml) and is washed with water (1×20 ml), 1 N aqueous hydrochloric acid (2×10 ml), saturated aqueous sodium bicarbonate (2×10 ml), and brine (1×10 ml). The organic layer is dried over anhydrous magnesium sulfate and concentrated to yield 6.

### p-TSA.H—R(NO₂)—W—NH₂ (7)

A solution of 6 (0.697 g, 1.381 mmol) in a mixture of TFA-DCM-water (10:40:0.5, 15 ml) is stirred at room temperature for 2-4 h. p-Toluenesulfonic acid, monohydrate (1.0 eq.) is added to the reaction mixture. After being stirred for 10 min, the mixture is concentrated by evaporation in vacuo and the residue is triturated with ether-hexane (1:1) to yield 2.

### Boc-(carba-4-Cl—Ff)-R(NO₂)—W—NH₂(8)

The procedure of preparing compound 6 is followed and 7 (0.5 mmol) is coupled with Boc-(carba-4-Cl—Ff)-OH (5, 0.5 mmol, 1.0 eq.) to yield 8.

### p-TSA.H-(carba-4-Cl—Ff)-R(NO₂)—W—NH₂(9)

The method of preparing compound 7 is followed and 9 is obtained from 8.

### Ac-(carba-4-Cl—Ff)-R(NO₂)—W—NH₂ (10)

The method of preparing compound 6 is followed and 9 (0.472 mmol) is coupled with acetic acid to yield crude 10 which is purified by preparative HPLC (C₁₈) to afford 10.

### Ac-(carba-Ff)-R—W—NH₂ (11)

A mixture of 10 (200 mg, 0.263 mmol), and 300 mg of 5% Pd-BaSO₄(unreduced) in MeOH-HOAc (10:1, 22 ml) is hydrogenated under 42 psi of hydrogen at room temperature for 17 h. The catalyst is removed by filtration through a pad of Celite which is washed with MeOH. The filtrate is concentrated to give crude 11 which is purified by preparative HPLC (C₁₈) and the product is further triturated with ether to yield 11 as a TFA salt.

### EXAMPLES 68 AND 69

### Syntheses of 3-(4-hydroxyphenyl)propanoyl-Atc-R-tryptamide (17) and 3-phenylpropanoyl-Atc-R-tryptamide (19)

These examples are prepared according to the plan presented in Scheme II:

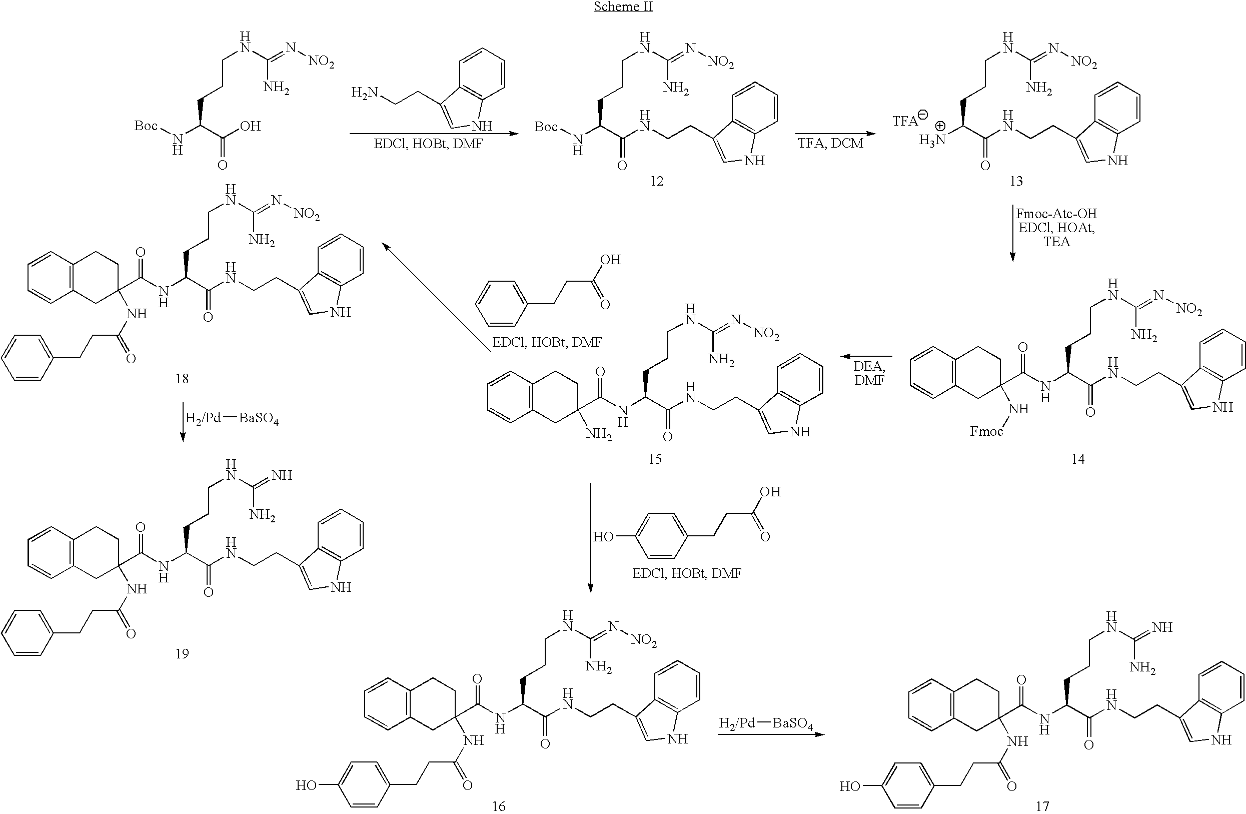

### Boc-R(NO₂)-tryptamide (12)

HOBt (1.0 eq.) and EDCI (0.949 g, 4.95 mmol, 1.1 eq.) are added sequentially to a well-stirred mixture of Boc-R(NO₂)—OH (1.437 g, 4.5 mmol) and tryptamine (0.721 g, 4.5 mmol) in DMF (20 ml) at 0° C. The resulting mixture is stirred for 30 minutes at 0° C. and then at room temperature for ˜20 h. The mixture is diluted with EtOAc (100 ml) and is then washed successively with water (2×20 ml), 5 % aqueous citric acid (3×10 ml), 5% aqueous sodium bicarbonate (2×10 ml), and brine (2×20 ml). The organic layer is dried over anhydrous sodium sulfate and concentrated by rotary evaporation to yield 12, which is used directly in the next step.

### H—R(NO₂)-tryptamide (13)

A solution of 12 (1.985 g, 4.3 mmol) in a mixture of trifluoroacetic acid-dichloromethane-water (10:40:0.5, 50 ml) is stirred at 0° C. for 15 minutes and then at room temperature for 16 h. The solvent is removed by rotary evaporation and the residue is co-evaporated with methanol (3×40 ml). The crude product is then purified by preparative HPLC to yield 13.

### Fmoc-Atc-R(NO₂)tryptamide (14)

HOAt (0.409 g, 3.005 mmol), EDCI (0.632 g, 3.306 mmol, 1.1 eq.), and TEA (1.1 eq.) are added sequentially to a well-stirred solution of 13 (1.086 g, 3.005 mmol) and Fmoc-Atc-OH (1.242 g, 3.005 mmol) in DMF (15 ml) at 0° C. The resulting mixture is stirred for 30 minutes at 0° C. and then at room temperature for 21 h. Work-up as described above for 12 gives crude 14. This material is used directly in the next step without further purification.

### H-Atc-R(NO₂)-tryptamide (15)

A solution of 14 (2.27 g, 3.0 mmol) in a mixture of DEA-DMF (1:9, 30 ml) is stirred at room temperature for 3 h. The solvent is removed by evaporation under reduced pressure and the residue is triturated with ether-hexane ( 1:1, 100 ml) to yield 15.

### 3-(4-hydroxyphenyl)propanoyl-Atc-R(NO₂)-tryptamide (16)

The procedure of preparing 12 is followed and 0.603 g of crude 16 is obtained from 15 (0.535 g, 1.0 mmol) and 3-(4-hydroxyphenyl)propanoic acid (0.166 g, 1.0 mmol). This crude product is purified by preparative HPLC to yield 16.

### 3-(4-hydroxyphenyl)propanoyl-Atc-R-tryptamide (17)

A mixture of 16 (0.270 g, 0.405 mmol) and 5% Pd-BaSO₄ (unreduced, 0.270 g) in MeOH—HOAc (10:1, 22 ml) is hydrogenated at room temperature and atmospheric pressure for 17 h. The catalyst is removed by filtration through a pad of celite and the filtrate is concentrated by rotary evaporation. The residue is purified by preparative HPLC and triturated with ether to yield 17.

### 3-phenylpropanoyl-Atc-R(NO₂)-tryptamide (18)

The procedure of preparing 16 is followed and 18 is obtained from 0.535 g (1.0 mmol) of 15 and 3-phenylpropanoic acid (0.150 g, 1.0 mmol).

### 3-phenylpropanoyl-Atc-R-tryptamide (19)

The procedure of preparing 17 is followed and 19 is obtained as a hydrated TFA salt from 0.270 mg of 18.

### EXAMPLE 70

### Synthesis of 3-Phenylpropanoyl-f-N(Me)R-tryptamide (25)

This example is prepared according to the plan presented in Scheme III:

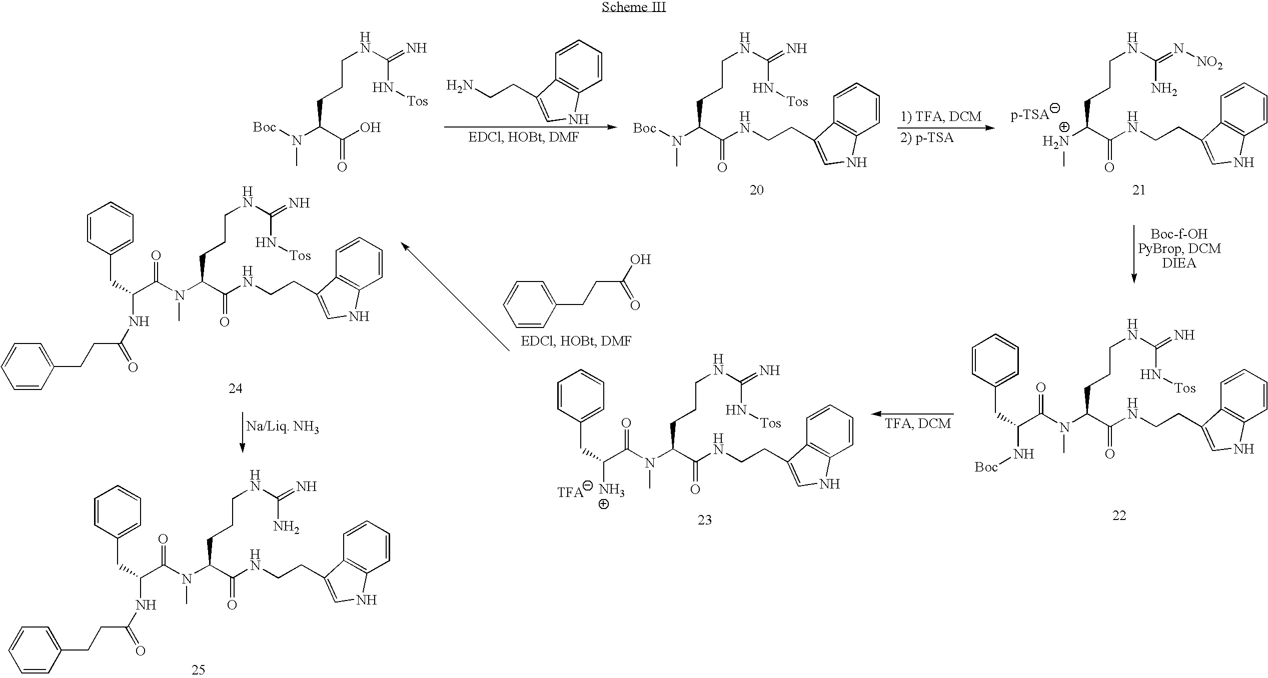

### Boc-N(Me)—R(Tos)-tryptamide (20)

The procedure of preparing 12 (Scheme I) is followed and crude 20 is obtained from 1.991 g (4.5 mmol) of Boc-N(Me)-R(Tos)-OH and 0.721 g (4.5 mmol) of tryptamine. This crude material is used in the next step without further purification.

### p-TSA.H—N(Me)—R(Tos)-tryptamide (21)

The procedure of preparing 7 (Scheme 0) is followed and 21 is obtained as a p-TSA salt from 2.631 g (4.5 mmol) of 20.

### Boc-f-N(Me)—R(Tos)-tryptamide (22)

DEA (0.310 ml, 1.772 mmol, 2.0 eq.) is added to a well-stirred mixture of 21 (0.4293 g, 0.886 mmol), Boc-f-OH (0.2351 g, 0.886 mmol), and PyBrop (0.3795 g, 0.886 mmol) in DCM (10 ml) at 0° C. The resulting reaction mixture is stirred at room temperature for 16 h and then is diluted with EtOAc (50 ml). The organic layer is washed successively with water (1×10 ml), 5% aqueous sodium bicarbonate (2×10 ml), and brine (2×10 ml) and is dried over anhydrous sodium sulfate. Removal of the dessicant and evaporation of the volatiles under reduced pressure yields crude 22.

### H-f-N(Me)—R(Tos)-tryptamide (23)

A solution of 22 (0.609 g, 0.833 mmol) dissolved in a mixture of TFA-DCM-water (10:40:0.5, 10 ml) is stirred at 0° C. for 1 h, and then is kept in a refrigerator at 4° C. for 72 h. The solvent is removed by evaporation in vacuo and the residue is then twice co-evaporated with DCM (10 ml ea.). The crude material so obtained is purified by preparative HPLC and triturated with ether to yield 23.

### 3-Phenylpropanoyl-f-N(Me)—R(Tos)-tryptamide (24)

TEA (0.209 mmol, 1.1 eq.) is added to a well-stirred mixture of 23 (0.120 g, 0.19 mmol), 3-phenylpropanoic acid (0.19 mmol), and EDCI (0.209 mmol, 1.1. eq) in DMF (3 ml) at 0° C. The resulting mixture is stirred at 0° C. for 0.5 h and then at room temperature for 16 h. The reaction mixture is diluted with EtOAc (30 ml) and is washed successively with water (2×3 ml), 5% aqueous citric acid (3×3 ml), 5% aqueous sodium bicarbonate (2×3 ml), and brine (2×5 ml). The organic layer is dried over anhydrous sodium sulfate, concentrated, and then triturated with ether to yield crude 24.

### 3-Phenylpropanoyl-f-N(Me)—R-tryptamide (25)

Compound 24 (100 mg, 0.13 mmol) is dissolved in liquid ammonia (25 ml) at −78° C. and small pieces of metallic sodium are added with vigorous magnetic stirring until a blue color persists for 2 h. The excess reagent is destroyed with ammonium acetate and the ammonia is removed by evaporation at room temperature. The residue is dissolved in methanol and the solution is filtered through a pad of silica gel that is subsequently washed with more methanol. The combined methanol filtrates are concentrated by rotary evaporation to give a crude material which is purified by preparative HPLC to yield 25 as a hydrated, TFA salt.

### EXAMPLES 71 THROUGH 75

### Syntheses of H-YfRW—NH₂(32), Bc-YfRW—NH₂ (34), CH₃(CH₂)₈CO—YfRW—NH₂ (36), Bc-YfRW-tryptamide (40), and CH₃(CH₂)₈CO—YfRW-tryptamide (42)

These examples are prepared according to the plan presented in Scheme V:

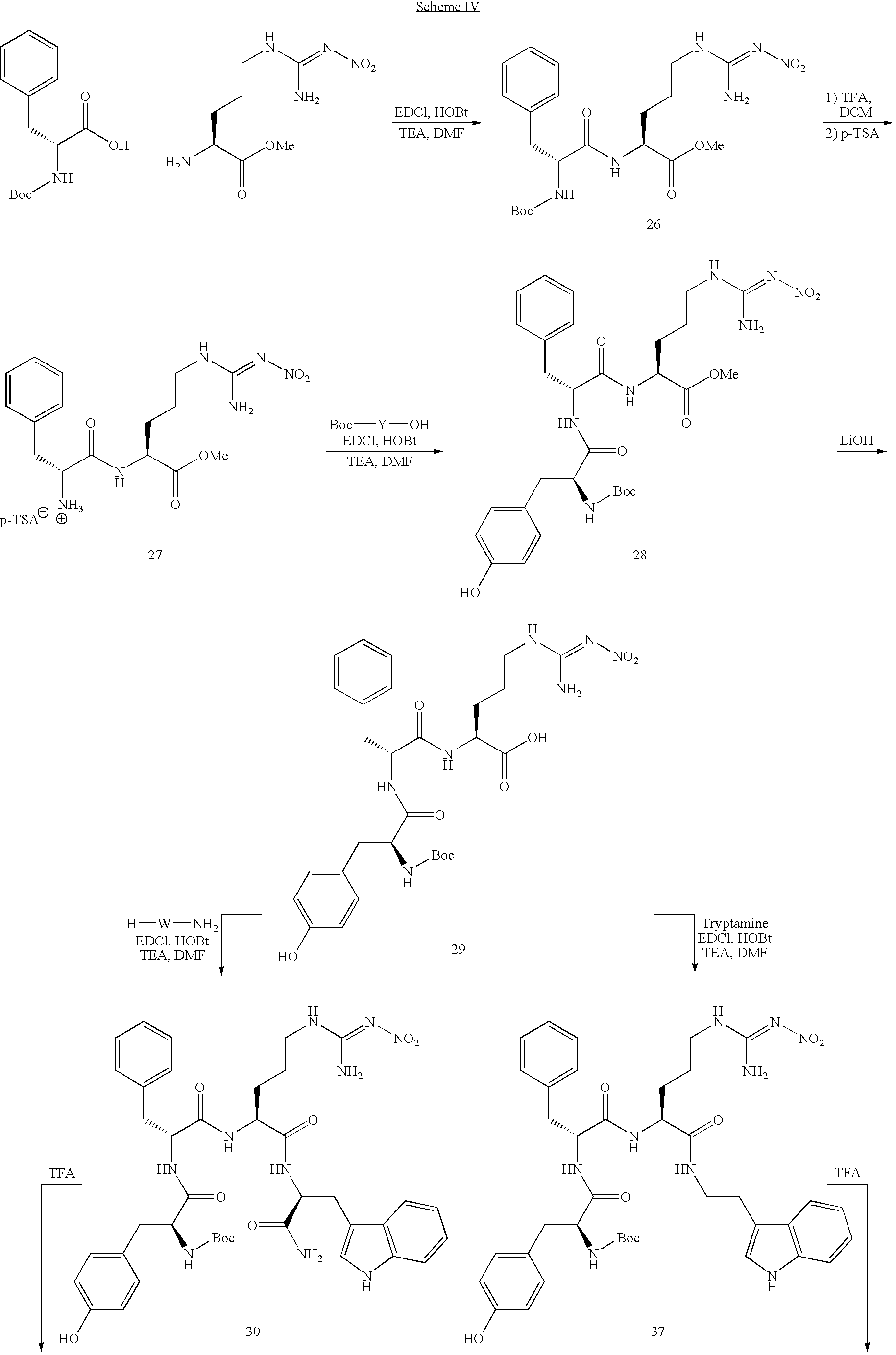

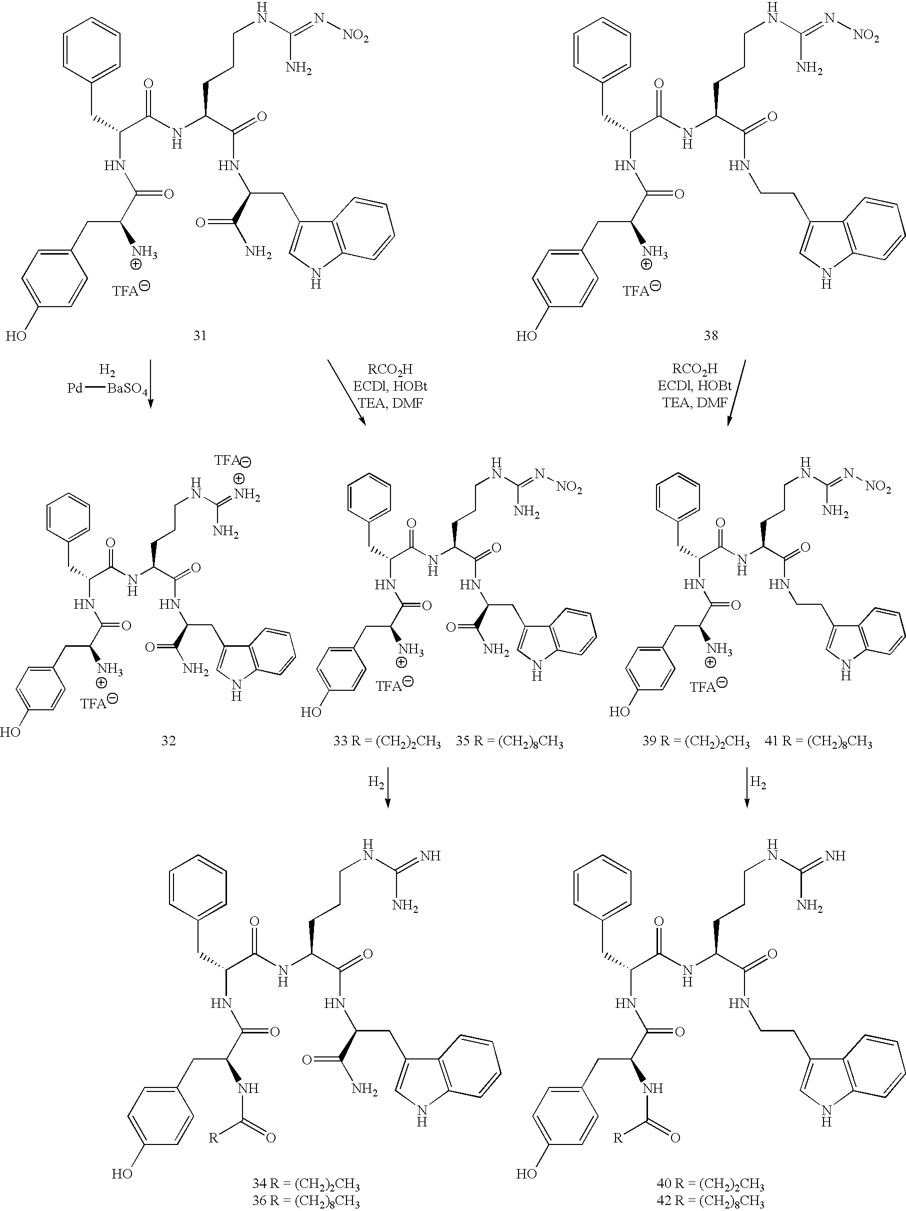

### Boc-fR(NO₂)—OMe(26)

Boc-f-OH (7.64 g, 28.8 mmol), H—W(NO₂)—OMe.HCl (6.72 g, 28.8 mmol), HOBt (3.94 g, 29.2 mmol), TEA (8 ml, 57.6 mmol) and DMF (110 ml, anhydrous) are combined and cooled at 0° C. and EDCI (5.89 g, 30.8 mmol) is added with stirring. After stirring at room temperature for 18 h, the mixture is concentrated by rotary evaporation, diluted with water (350 ml), and extracted with EtOAc (4×80 ml). The combined organic extract is washed with aqueous 1 N HCl (3×60 ml), saturated aqueous NaHCO₃ (2×50 ml), brine (45 ml), and is then dried with anhydrous Na₂SO₄. After filtration, the filtrate is concentrated by rotary evaporation, co-evaporated with ether (50 ml), and dried in vacuo to give 3.

### p-TSA.H-fR(NO₂)—OMe (27)

TFA (6 ml) is added to a solution of Boc-fR(NO₂)—OMe (26) (1.49 g, 3.1 mmol) in DCM (20 ml) at 0° C. After stirring at 0° C. for 30 min., the reaction mixture is stirred at room temperature for 5 h. The solvent is removed in vacuo, the residue co-evaporated with ether (25 ml), and the resulting residue is dissolved in methanol (25 ml). p-toluenesulfonic acid monohydrate (0.6 g, 3.1 mmol) is added, the mixture stirred for 5 min. at room temperature, the volatiles removed in vacuo, and the residue co-evaporated with ether (2×25 ml) to give 27.

### Boc-YfR(NO₂)—OMe (28)

p-TSA.H-fR(NO₂)—OMe (27, 3.46 g, 6 mmol), Boc-Y—OH (1.72 g, 6.12 mmol), HOBt (0.83 g, 6.18 mmol), TEA (1.6 ml, 12 mmol) and DMF (25 ml, anhydrous) are combined and cooled at 0° C. and EDCI (1.1 g, 6.3 mmol) is added with stirring. After stirring at room temperature for 16 h, the mixture is concentrated by rotary evaporation, diluted with water (110 ml), and extracted with EtOAc (4×23 ml). The combined organic extract is washed with aqueous 1 N HCl (3×20 ml), saturated aqueous NaHCO₃ (2×25 ml), brine (25 ml), and is then dried with anhydrous Na₂SO₄. After filtration, the filtrate is concentrated by rotary evaporation to give 5.

### Boc-YfR(NO₂)—OH (29)

A mixture of Boc-YfR(NO₂)—OCH₃ (28, 2.64 g, 4.1 mmol), LiOH (0.113 g, 4.72 mmol), water (0.4 ml) and MeOH (10 ml) is stirred at room temperature for 6 h. After removal of the solvent, the residue is dissolved in a minimum amount of water. 1 N HCl (ca. 4.7 ml) is added to neutralize the mixture to pH 5-6. Filtration of the solid and drying in vacuo gives 29.

### Boc-YfR(NO₂)W—NH₂ (30)

Boc-YfR(NO₂)—OH (29, 0.99 g, 1.56 mmol), H—W—NH₂HCl (0.43 g, 1.8 mmol), HOBt (0.25 g, 1.84 mmol), TEA (0.55 ml, 3.9 mmol) and DMF (20 ml, anhydrous) are combined and cooled at 0° C. and EDCI (0.36 g, 1.87 mmol) is added with stirring. After stirring at room temperature for 16 h, the mixture is concentrated by rotary evaporation, diluted with water (120 ml), and extracted with EtOAc (4×25 ml). The combined organic extract is washed with aqueous 1 N HCl (3×20 ml), saturated aqueous NaHCO₃ (2×25 ml), brine (20 ml), and is then dried with anhydrous Na₂SO₄. After filtration, the filtrate is concentrated by rotary evaporation, co-evaporated with ether (20 ml), and dried in vacuo to give 30.

### H—YfR(NO₂)-Trp-NH₂ (31)

TFA (2 ml) is added to a solution of peptide 30 (0.95 g, 1.16 mmol) in DCM (6 ml) at 0° C. After stirring at 0° C. for 30 min, the reaction mixture is stirred at room temperature for 5 h. The solvent is removed by rotary evaporation and the resulting residue is purified by preparative HPLC to give 31.

### H—YfRW—NH ₂(32)

Peptide 31 (0.25 g, 0.35 mmol), 5% Pd/BaSO₄ (0.25 g, unreduced) and MeOH (15 ml) are combined and hydrogenated under 40 psi of hydrogen at room temperature for 48 h. After filtration, the filtrate is concentrated by rotary evaporation and the residue purified by preparative HPLC to yield H—YfRW—NH₂ (32).

### Bc-YfR(NO₂)W—NH₂ (33)

The procedure of preparing 28 is used, followed by purification by preparative HPLC and 33 is obtained from H—YfR(NO₂)W—NH₂ (31, 0.26 g, 0.37 mmol), butyric acid (0.037 g, 0.42 mmol), HOBt (0.059 g, 0.43 mmol), TEA (0.1 ml, 0.74 mmol), EDCI (0.084 g, 0.44 mmol), and DMF (12 ml).

### Bc-YfRW—NH₂ (34)

The procedure of preparing 32 is followed and 34 is obtained from 33 (0.16 g, 0.2 mmol), 5% Pd/BaSO₄ (0.15 g, unreduced), MeOH (12 ml), and TFA (0.1 ml).

### CH₃(CH₂)₈CO—YtR(NO₂)W—NH₂ (35)

The procedure of preparing 28 is used followed by purification by preparative HPLC and 35 is obtained from H—YfR(NO₂)W—NH₂ (31, 0.26 g, 0.36 mmol), decanoic acid (0.071 g, 0.41 mmol), HOBt (0.057 g, 0.42 mmol), TEA (0.1 ml, 0.73 mmol), EDCI (0.083 g, 0.43 mmol), and DMF (12 ml).

### CH₃(CH₂)₈CO—YfRW—NH₂ (36)

The procedure of preparing 31 is followed and 36 is obtained from 35 (0.2 g, 0.23 mmol), 5% Pd/BaSO₄ (0.18 g, unreduced), MeOH (12 ml), and TFA (0.1 ml).

### Boc-YfR(NO₂)-tryptamide (37)

The procedure of preparing 30 is used and 37 is obtained from Boc-YfR(NO₂)—OH (29, 0.95 g, 1.5 mmol), tryptamine (0.276 g, 1.73 mmol), HOBt (0.239 g, 1.77 mmol), TEA (0.5 ml, 3.6 mmol), EDCI (0.34 g, 1.8 mmol), and DMF (18 ml).

### H—YfR(NO₂)tryptamide (38)

Peptide 30 (0.98 g, 1.27 mmol) in DCM (6 ml) and a solution of anisole-TFA-DCM (1:8:9, 6 ml) are mixed at 0° C. and stirred with ice-cooling for 20 min. The mixture is then allowed to stir at room temperature overnight. The solvent is removed and the resulting residue is co-evaporated with ether (2×25 ml) to give crude 38.

### Bc-YfR(NO₂)-tryptamide (39)

The procedure of preparing 28 is used followed by purification by preparative HPLC and 39 is obtained from 38 (0.28 g, 0.41 mmol), butyric acid (0.042 g, 0.47 mmol), HOBt (0.066 g, 0.49 mmol), TEA (0.12 ml, 0.83 mmol), EDCI (0.094 g, 0.49 mmol), and DMF (10 ml).

### Bc-YfR(NO₂)-tryptamide (40)

The procedure of preparing 32 is followed and 40 is obtained from 39 (0.19 g, 0.25 mmol), 5% Pd/BaSO₄ (0.18 g, unreduced), MeOH (12 ml), and TFA (0.1 ml).

### CH₃(CH₂CO—YfR(NO₂)-tryptamide (41)

The procedure of preparing 28 is used followed by purification by preparative HPLC and 41 is obtained from 38 (0.27 g, 0.4 mmol), decanoic acid (0.081 g, 0.47 mmol), HOBt (0.065 g, 0.48 mmol), TEA (0.11 ml, 0.81 mmol), EDCI (0.093 g, 0.49 mmol), and DMF (12 ml).

### CH₃(CH₂)₈CO—YfRW-tryptamide (42)

The procedure of preparing 32 is followed and 42 is obtained from 41 (0.21 g, 0.25 mmol), 5% Pd/BaSO₄ (0.18 g, unreduced), MeOH (12 ml), and TFA (0.1 ml).

### EXAMPLES 76 THROUGH 81

### Syntheses of Ac—YfRW—OMe (47), Ac—YfRW—NHCH₃ (49), AcYfRWSar-NH₂ (50), 3-(4-OH-Ph)propanoyl-fRW—NHCH₃ (53), 3-(4-OH-Ph)propanoyl-fRW—NH(CH₂)₂OH (54), and 3-(4- OH-Ph)propanoyl-fRW-NH(CH₂)₂OH (55)

These examples are prepared according to the plan presented in Scheme V:

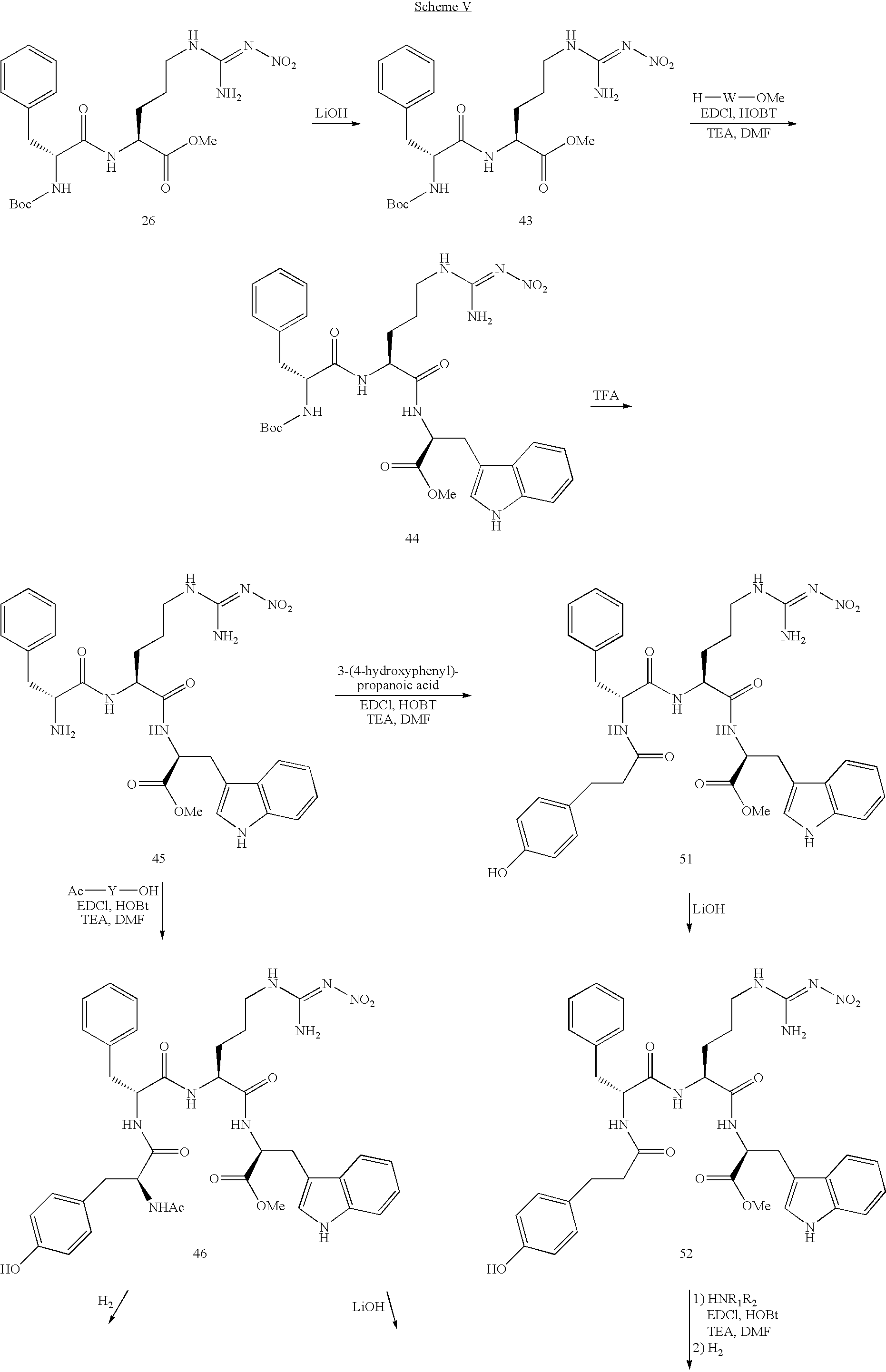

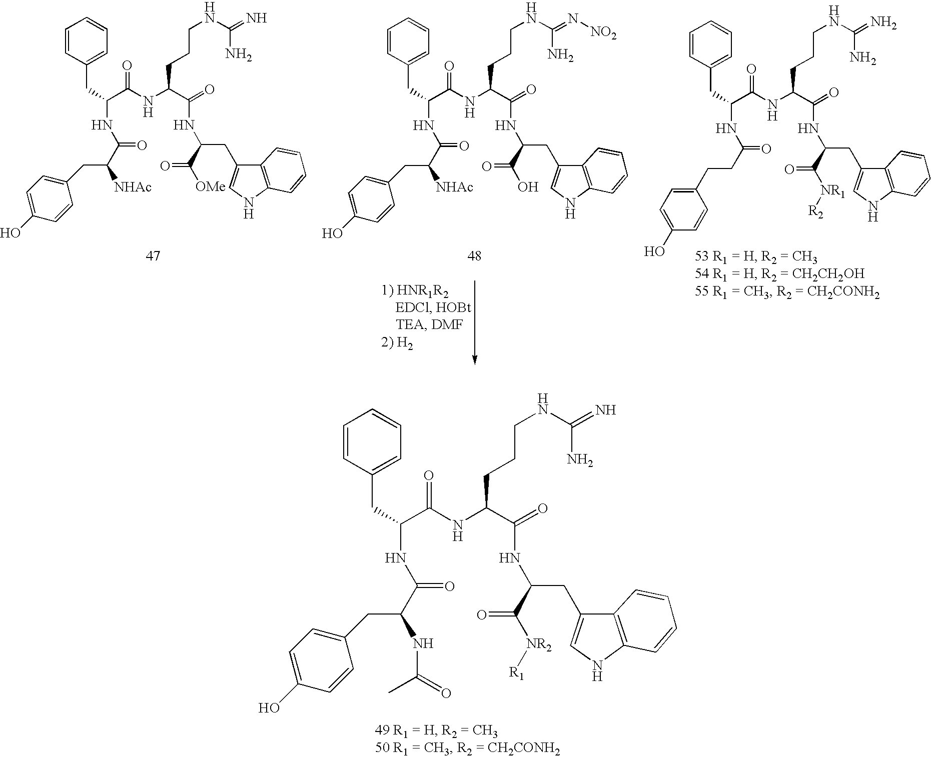

### Boc-fR(NO₂)—OH (43)

The procedure of preparing 29 is followed and 43 is obtained from Boc-fR(NO₂)—OMe (26, 2.8 g, 5.8 mmol), LiOH (0.27 g, 1 mmol) and MeOH (15 ml).

### Boc-fR(NO₂)W—OMe (44)

The procedure of 26 is followed and of 44 is obtained from Boc-fR(NO₂)—OH (43, 4.22 g, 9.06 mmol), H—W—OMe.HCl (2.65 g, 10.4 mmol), HOBt (1.44 g, 10.7 mmol), TEA (3.15 ml, 22.6 mmol), EDCI (2.08 g, 10.9 mmol), and DMF (55 ml).

### H-fR(NO₂)W—OMe (45)

TFA (12 ml) is added to a solution of 44 (5 g, 7.5 mmol) in DCM (30 ml) at 0° C. After stirring at 0° C. for 1 h, the reaction mixture is stirred at room temperature for 16 h. The solvent is removed in vacuo, the residue co-evaporated with ether (2×35 ml), and the product dried in vacuo to give crude 45.

### Ac—YfR(NO₂)W—OMe (46)

The procedure of preparing 28 is followed and crude 46 is collected from H-fR(NO₂)W—OMe (45, 3.3 g, 5.8 mmol), Ac—Y—OH (1.5 g, 6.7 mmol), HOBt (0.93 g, 6.9 mmol), TEA (1.7 ml, 12.2 mmol), EDCI (1.34 g, 7 mmol), and DMF (25 ml).

### Ac—YfRW—OMe (47)

The procedure of preparing 32 is followed and 47 is obtained from 46 (0.2 g, 0.26 mmol), 5% Pd/BaSO₄ (0.19 g, unreduced), MeOH (15 ml), and TFA (0.1 ml).

### Ac—YfR(NO₂)W—OH (48)

The procedure of preparing 29 is used followed by purification by preparative HPLC and 48 is obtained from Ac—YfR(NO₂)W—OMe (46, 0.96 g, 1.25 mmol), LiOH (0.063 g, 2.6 mmol), water (0.4 ml), and MeOH (6 ml).

### Ac—YfRW-NHCH₃ (49)

The procedure of preparing 30 is followed, except for the co-evaporation step, and a coupling product is obtained from Ac—YfR(NO₂)W—OH (48, 0.3 g, 0.39 mmol), methyl amine (0.014 g, 0.45 mmol), HOBt (0.058 g, 0.43 mmol), TEA (0.092 ml, 0.91 mmol), EDCI (0.089 g, 0.47 mmol), and DMF(12 ml). This product is purified by preparative HPLC and is then hydrogenated using the procedure of preparing 32 with 5% Pd/BaSO₄ (0.17 g, unreduced) in MeOH (12 ml) to give 49.

### Ac—YfRWSar-NH₂ (50)

The procedure of preparing 30 is followed, except for the co-evaporation step, and a coupling product is obtained from Ac—YfR(NO₂)W—OH (19, 0.3 g, 0.39 mmol), sarcosine amide, HCl (0.05 g, 0.4 mmol), HOBt (0.058 g, 0.43 mmol), TEA (0.092 ml, 0.91 mmol), EDCI (0.089 g, 0.47 mmol), and DMF (12 ml). The product is purified by preparative RPLC and is then hydrogenated using the procedure of preparing 32 with 5% Pd/BaSO₄ (0.18 g, unreduced) in MeOH (12 ml) to give 50.

### 3-(4-OH-Ph)propanoyl-fRW—OCH₃ (51)

The procedure of preparing 26 is used followed by purification by preparative HPLC to give 51 from H-fR(NO₂)W—OCH₃ 45 (2.1 g, 3.7 mmol), 3-(4-hydroxyphenyl)propanoic acid (0.71 g, 4.3 mmol), HOBt (0.59 g, 4.4 mmol), TEA (1.1 ml, 0.91 mmol), EDCI (0.85 g, 4.5 mmol), and DMF (25 ml).

### 3-(4-OH-Ph)propanoyl-fRW—OH (52)

The procedure of preparing compound 29 is followed to give 52 from 51 (1.15 g, 1.61 mmol), LiOH (0.081 g, 3.38 mmol), water (0.4 ml) and MeOH (8 ml).

### 3-(4-OH-Ph)propanoyl-fRW-NHCH₃ (53)

The procedure of preparing compound 30 is followed to give a coupling product from 52 (0.3 g, 0.42 mmol), methylamine (0.015 g, 0.49 mmol), HOBt (0.064 g, 0.47 mmol), TEA (0.14 ml, 0.98 mmol), EDCI (0.098 g, 0.51 mmol), and DMF (12 ml). This product is purified by preparative HPLC and hydrogenated using the procedure of 32 with 5% Pd/BaSO₄ (0.16 g) and MeOH (12 ml) to give 53.

### 3-(4-OH-Ph)propanoyl-fRW—NHCH₂CH₂OH (54)

The procedure of preparing compound 30 is followed to give a coupling product from 52 (0.3 g, 0.42 mmol), ethanolamine (0.031 g, 0.51 mmol), HOBt (0.066 g, 0.49 mmol), TEA (0.14 ml, 1 mmol), EDCI (0.1 g, 0.52 mmol), and DMF (12 ml). This product is purified by preparative HPLC and hydrogenated using the procedure of preparing compound 32 with 5% Pd/BaSO₄ (0.18 g) and MeOH (12 ml) to give 54.

### 3-(4-OH-Ph)propanoyl-fRWSar-NH₂ (55)

The procedure of preparing compound 30 is followed to give a coupling product from 52 (0.3 g, 0.42 mmol), sarcosine amide HCl (0.061 g, 0.49 mmol), HOBt (0.068 g, 0.5 mmol), TEA (0.15 ml, 1.1 mmol), EDCI (0.098 g, 0.51 mmol), and DMF (12 ml). This product is purified by preparative HPLC and hydrogenated using the procedure of preparing compound 32 with 5% Pd/BaSO₄ (0.18 g) and MeOH (12 ml) to give.

### EXAMPLES 82 THROUGH 84

Syntheses of Bc-YfRW—NHCH₃ (63), Bc-YfRW—NH(CH₂)₂OH (64), and BcYfRW—N(CH₃)(CH₂)₂OH (65)

These examples are prepared according to the plan presented in Scheme VI:

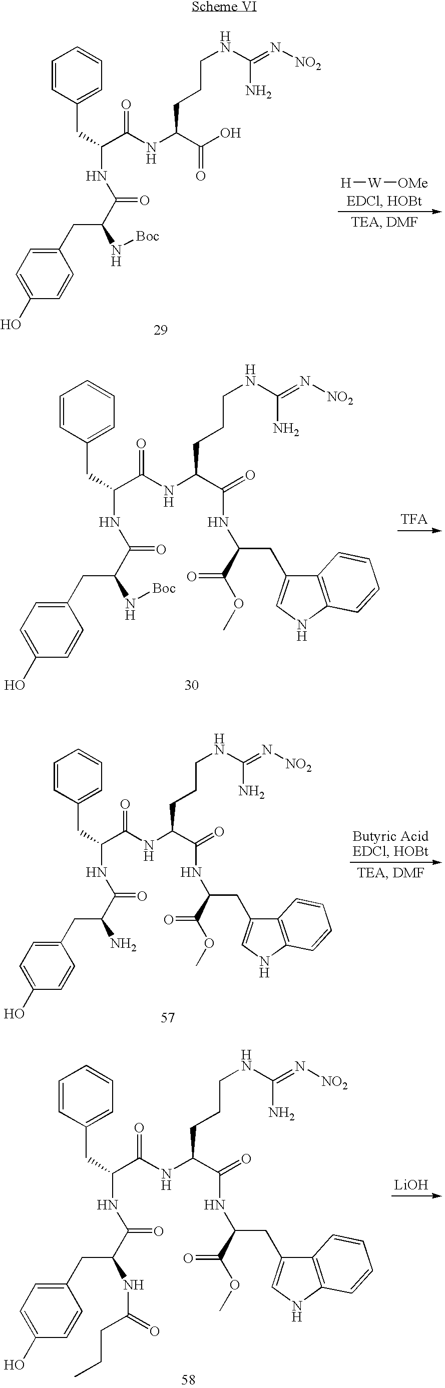

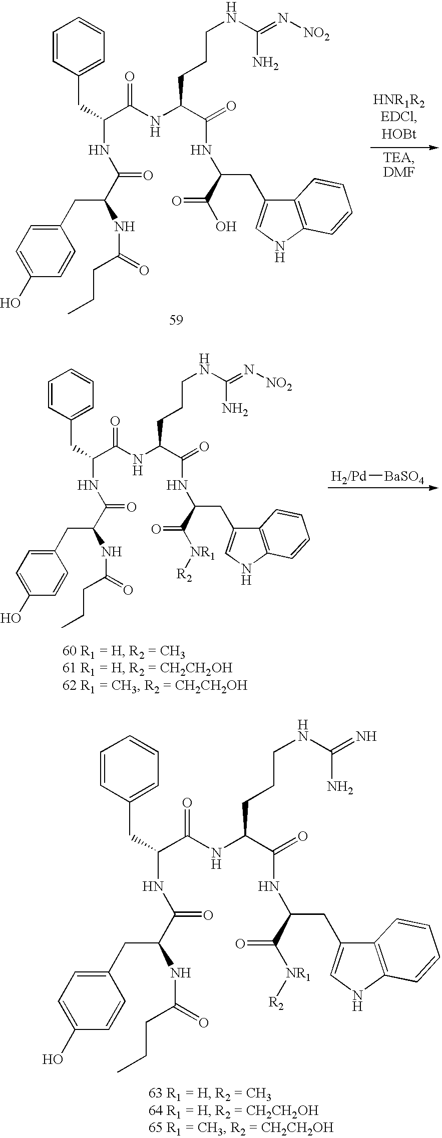

### Boc-YfRW—OCH₃ (56)

The procedure of preparing compound 28 is followed and 2.1 g (95%) of 56 is obtained from Boc-YfR(NO₂)—OH (29, 1.7 g, 2.6 mmol), H—W—OCH₃.HCl (0.68 g, 2.7 mmol), HOBt (0.42 g, 3.1 mmol), TEA (0.8 ml, 5.8 mmol), EDCI (0.63 g, 3.3 mmol), and DMF(25 ml).

### H—YfRW—OCH₃ (57)

The procedure of preparing compound 38 is followed and 57 is obtained from 56 (2.1 g, 2.5 mmol) and a solution of anisole-TFA-DCM (1:8:9, 18 ml).

### Bc-YfRW—OCH₃ (58)

The procedure of preparing compound 28 is followed to give a crude coupling product from H- YfR(NO₂)W—OCH₃ (57,1.21 g, 1.65 mmol), butyric acid (0.18 g, 2 mmol), HOBt (0.26 g, 1.95 mmol), TEA (0.57 ml, 4.1 mmol), EDCI (0.394 g, 2.06 mmol), and DMF (25 ml). This product is purified by preparative HPLC to give 58.

### Bc-YfRW—OH (59)

The procedure of preparing compound 29 is followed to give 59 from 58 (0.1 g, 0.13 mmol), LiOH (0.004 g, 0.15 mmol), water (0.2 ml) and MeOH (2 ml).

### Bc-YfRW—NHCH₃ (63)

The procedure of preparing compound 28 is followed to give a coupling product 60 from 59 (0.1 g, 0.125 mmol), methyl amine (0.006 ml, 0.15 mmol), HOBt (0.02 g, 0.15 mmol), TEA (0.04 ml, 0.3 mmol), EDCI (0.03 g, 0.16 mmol), and DMF (3 ml). This product is hydrogenated according to the procedure of preparing compound 32 with 5% Pd/BaSO₄ (0.1 g, unreduced) in MeOH (6 ml) to give 63.

### Bc-YfR(NO₂)W—NH(CH₂)₂0H (61)

The procedure of preparing compound 28 is used followed by purification by HPLC to give 61 from 59 (1.3 g, 1.65 mmol), ethanolamine (0.12 ml, 1.9 mmol), HOBt (0.263 g, 1.95 mmol), TEA (0.55 ml, 4 mmol), EDCI (0.38 g, 2 mmol), and DMF (20).

### Bc-YfRW—NH(CH₂)₂OH (64)

The procedure of preparing compound 32 is followed and 64 is obtained as a solid from 61 (0.22 g, 0.26 mmol), 5% Pd/BaSO₄ (0.2 g, unreduced), MeOH (12 ml), and TFA (0.1 ml).

### Bc-YfR(NO₂)W—N(CH₃)(CH₂)₂OH (62)

The procedure of preparing compound 28 is used followed by purification by HPLC to give 62 from 59 (0.26 g, 0.33 mmol), N-methylethanolamine (0.032 ml, 0.4 mmol), HOBt (0.053 g, 0.39 mmol), TEA (0.11 ml, 0.83 mmol), EDCI (0.076 g, 0.4 mmol), and DMF (10 ml).

### Bc-YfRW—N(CH₃)(CH₂)₂OH (65)

The procedure of preparing compound 32 is followed and 65 is obtained from 62 (0.16 g, 0.19 mmol), 5% Pd[BaSO₄ (0.19 g, unreduced), MeOH (10 ml), and TFA (0.1 ml).

### EXAMPLE 85

### Synthesis of 3-(4-OHPh)propanoyl-YfRW—NH₂ (69)

This example is prepared according to the plan presented in Scheme VII:

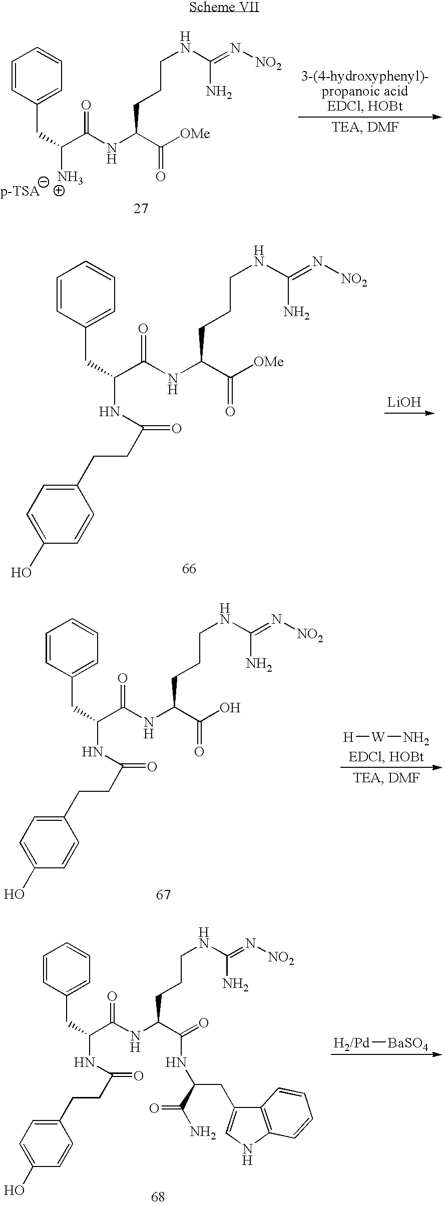

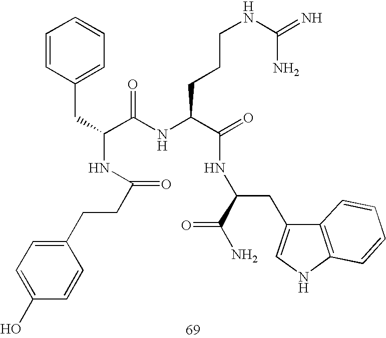

### 3-(4-OHPh)propanoyl-fR(NO₂)—OCH₃ (66)

The procedure of preparing compound 28 is used followed by purification by preparative HPLC to give 66 from 27 (1.5 g, 2.6 mmol) 3-(4-hydroxyphenyl)propanoic acid (0.48 g, 2.9 mmol), HOBt (0.43 g, 3.2 mmol), TEA (0.91 ml, 6.6 mmol), EDCI (0.63 g, 3.3 mmol), and DMF (28 ml).

### 3-(4-OHPh)propanoyl-fR(NO₂)—OH (67)

The procedure of preparing compound 29 is followed to give 67 from 66 (0.33 g, 0.63 mmol), LiOH (0.029 g, 1.2 mmol), water (0.4 ml) and MeOH (5 ml).

### 3-(4-OHPh)propanoyl-fR(NO₂)W—NH (68)

The procedure of preparing compound 28 is used followed by purification by preparative HPLC to give 68 from 67 (0.32 g, 0.62 mmol), H—W—NH₂HCl (0.16 g, 0.68 mmol), HOBt (0.1 g, 0.73 mmol), TEA (0.2 ml, 1.6 mmol), EDCI (0.14 g, 0.75 mmol), and DMF (13 ml).

### 3-(4-OHPh)propanoyl-fRW—NH₂ (69)

The procedure of preparing compound 32 is followed and 69 is obtained from 68 (0.14 g, 0.2 mmol), 5% Pd/BaSO₄ (0.12 g, unreduced), MeOH (9 ml), and TFA (0.1 ml).

### EXAMPLE 86

### Synthesis of Ac—YfR-tryptamide (73)

This example is prepared according to the plan presented in Scheme VIII:

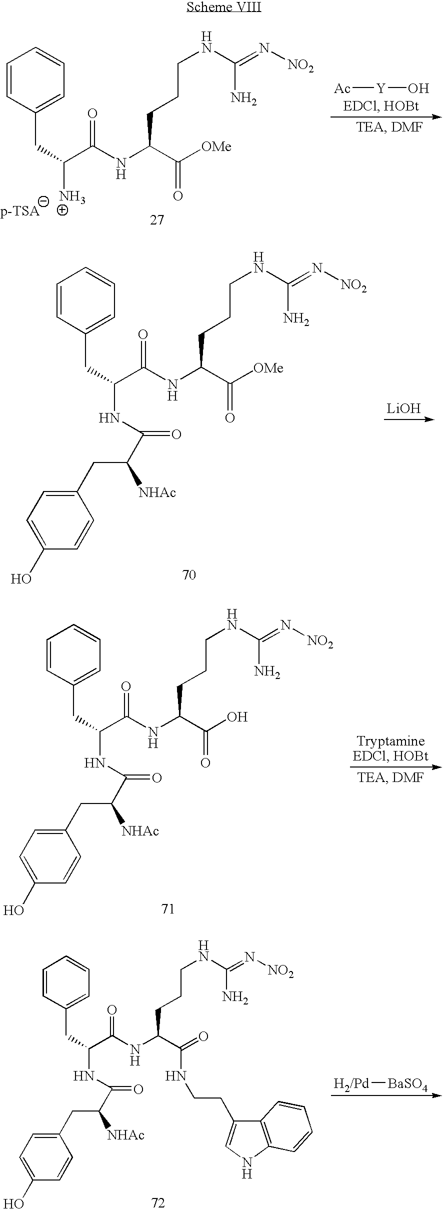

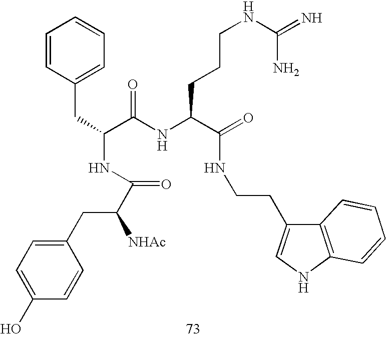

### Ac—YfR(NO₂)—OCH₃ (70)

The procedure of preparing compound 28 is used followed by purification by preparative HPLC to give 70 from 27 (0.36 g, 0.63 mmol), Ac—Y—OH (0.14 g, 0.63 mmol), HOBt (0.1 g, 0.74 mmol), TEA (0.17 ml, 1.2 mmol), EDCI (0.14 g, 0.76 mmol), and DMF (12 ml).

### Ac—YfR(NO₂)—OH (71)

The procedure of preparing compound 29 is followed with an additional co-evaporation step from THF (5 ml) to give a mixture of 71 and two equivalents of LiCl from 70 (0.08 g, 0.14 mmol), LiOH (0.007 g, 0.28 mmol), water (0.2 ml), and MeOH (4 ml).

### Ac—YfR-tryptamide (73)

The procedure of preparing compound 28 is followed to give a crude coupling product 72 from the mixture of Ac—YfR(NO₂)—OH/2LiCl (71, 0.091 g, 0.136 mmol), tryptamine (0.025 ml, 0.15 mmol), HOBt (0.021 g, 0.15 mmol), TEA (0.1 ml, 0.7 mmol), EDCI (0.03 g, 0.16 mmol), and DMF (6 ml). This product is hydrogenated using the procedure of preparing compound 32 using 5% Pd/BaSO₄(0.2 g, unreduced) and MeOH (8 ml) to give 73 as a solid.

### EXAMPLE 87

### Synthesis of Hydrocinnamoyl-fRW—NH₂ (77)

This example is prepared according to the plan presented in Scheme IX:

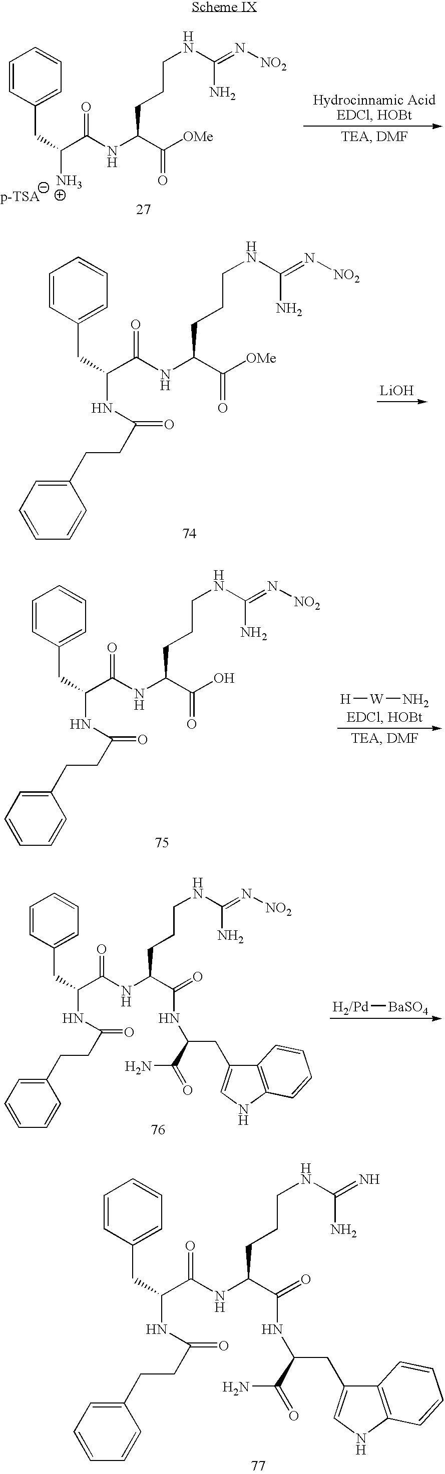

### Hydrocinnamoyl-fR(NO₂)—OCH₃ (74)

The procedure of preparing compound 28 is followed to give 74 from 27 (0.25 g, 0.43 mmol), hydrocinnamic acid (0.068 g, 0.45 mmol), HOBt (0.068 g, 0.5 mmol), TEA (0.16 ml, 1.1 mmol), EDCI (0.097 g, 0.51 mmol), and DMF (20 ml).

### Hydrocinnamoyl-fW(NO₂)—OH (75)

The procedure of preparing compound 29 is followed to give 75 from 74 (0.22 g, 0.43 mmol), LiOH (0.014 g, 0.56 mmol), water (0.3 ml) and MeOH (8 ml).

### Hydrocinnamoyl-fRW—NH₂ (77)

The procedure of preparing compound 28 is followed to give a crude coupling product 76 from 75 (0.2 g, 0.4 mmol), H—W—NH₂.HCl (0.1 g, 0.42 mmol), HOBt (0.068 g, 0.5 mmol), TEA (0.14 ml, 1 mmol), EDCI (0.1 g, 0.52 mmol), and DMF (12 ml). This product is hydrogenated using the procedure of preparing compound 32 using 5% Pd/BaSO₄ (0.2 g, unreduced) and MeOH (10 ml) to give 77.

### EXAMPLE 88

### Synthesis of 3-(4-OHPh)propanoyl-(4-F-f)RW—NH₂ (82)

This example is prepared according to the plan presented in Scheme X:

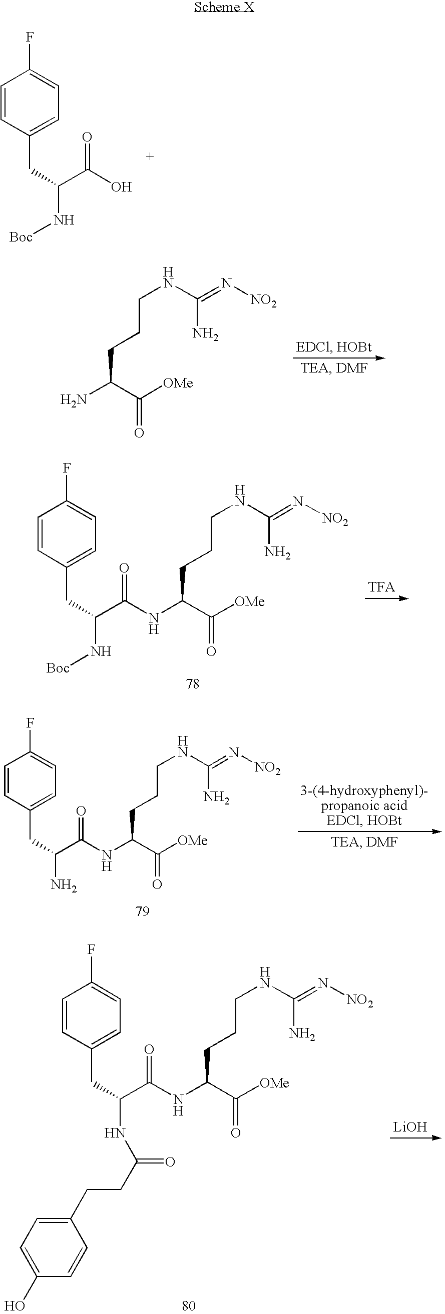

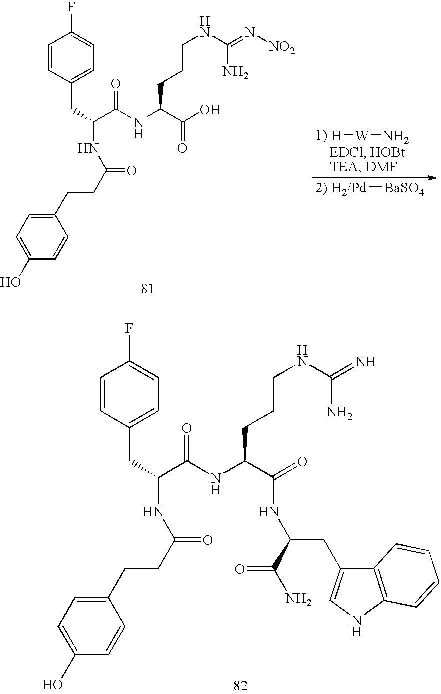

### Boc-(4-F-f)R(NO₂)—OMe (78)

The procedure of preparing compound 26 is followed to give 78 from Boc-(4-F-f)-OH (1.5 g, 5.3 mmol), H—R(NO₂)—OMe.HCl (1.46 g, 5.4 mmol), HOBt (0.87 g, 6.4 mmol), TEA (1.86 ml, 13.3 mmol), EDCI (1.33 g, 6.9 mmol), and DMF (35 ml).

### H-(4-F-f)R(NO₂)—OMe (79)

TFA (4 ml) is added to a solution of 78 (2.48 g, 5 mmol) in DCM (20 ml) at 0° C. The mixture is stirred with ice-cooling for 30 min. and then is stirred at room temperature overnight. The solvent is removed, the residue co-evaporated with ether (2×30 ml), and the resulting residue is dried under vacuum to give 79 as a solid.

### 3-(4-OHPh)propanoyl-(4-F-f)R(NO₂)—OMe (80)

The procedure of preparing compound 26 is followed to give 80 from 79 (2 g, 5 mmol), 3-(4-hydroxyphenyl)-propanoic acid (0.92 g, 5.5 mmol), HOBt (0.8 g, 5.9 mmol), TEA (1.7 ml, 12.6 mmol), EDCI (1.15 g, 6 mmol), and DMF (25 ml).

### 3-(4-OHPh)propanoyl-(4-F-f)R(NO₂)—OH (81)

The procedure of preparing compound 29 is followed to give 81 from 80 (0.75 g, 1.4 mmol), LiOH (0.065 g, 2.7 mmol), water (0.4 ml), and MeOH (8 ml).

### 3-(4-OHPh)propanoyl-(4-F-f)R(NO₂)W—NH₂ (82)

The procedure of preparing compound 30 is used followed by purification by preparative HPLC to give a coupling product from 81 (0.4 g, 0.75 mmol), H—W—NH₂.HCl (0.2 g, 0.82 mmol), HOBt (0.12 g, 0.9 mmol), TEA (0.26 ml, 1.9 mmol), EDCI (0.17 g, 0.9 mmol), and DMF (20 ml).). This product is hydrogenated using the procedure of preparing compound 32 using 5% Pd/BaSO₄ (0.25 g, unreduced) and MeOH (15 ml) to give 82.

### EXAMPLES 89-91

### Syntheses of 3-(4-OHPh)propanoyl-fR-tryptamide (88), 3-(2-OHPh)propanoyl-fR-tryptamide (89), and Hydrocinnamoyl-fR-tryptamide (90)

These examples are prepared according to the plan presented in Scheme XI:

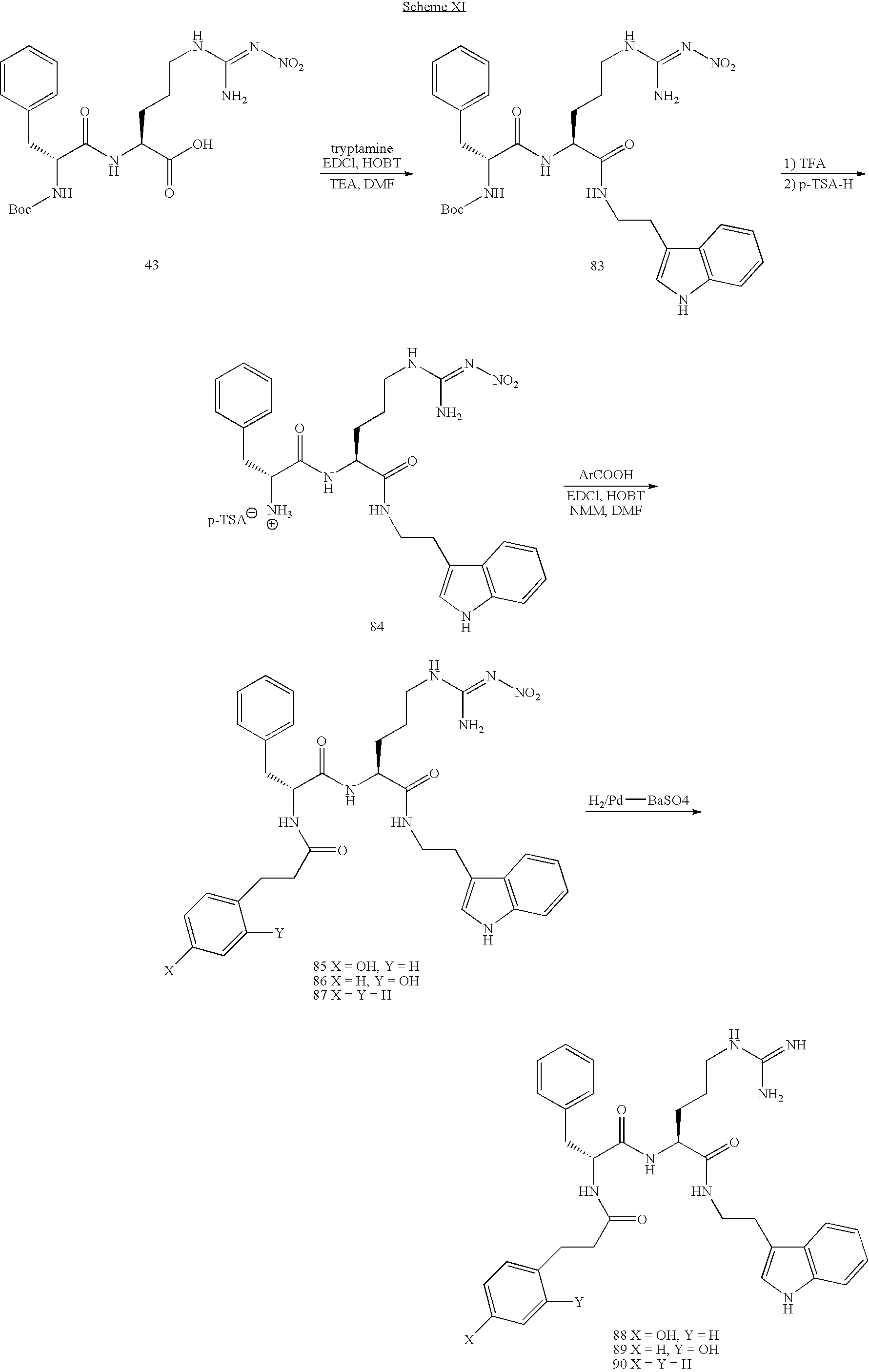

### Boc-fR(NO₂)-tryptamide (83)

Boc-fR(NO₂)—OH (43,507.9 mg, 1.09 mmol), tryptamine (172.9 mg, 1.08 mmol), HOBt (163.3 g, 1.08 mmol), TEA (0.16 ml, 0.12 mmol) and DMF (6 ml, anhydrous) are combined and cooled at 0° C. and EDCI (226.9 mg, 1.18 mmol) is added with stirring. After stirring at 0° C. for 45 min the ice bath is removed and the mixture is warmed and stirred at room temperature for 14.5 h. The mixture is diluted with EtOAc (20 ml) and washed with aqueous 2 N HCl (3×5 ml), the aqueous acid layer is back-extracted with EtOAc (1×10 ml), and the combined EtOAc layers are washed with 1 M NaHCO₃ (3×5 ml) and brine (10 ml). The organic extract is then dried with anhydrous Na₂SO₄, the dessicant is removed by filtration, and the filtrate is concentrated by rotary. This solid is dried in vacuo to give 83.

### p)-TSA.H-fR(NO₂)-tryptamide (84)

TFA (3 ml) is added to a solution of Boc-fR(NO₂)-tryptamide (83) (0.58 g, 0.95 mmol) in DCM (6 ml) at 0° C. After stirring at 0° C. for 30 min., the reaction mixture is stirred at room temperature for 1.25 h. p-Toluenesulfonic acid monohydrate (176.2 mg, 0.93 mmol) is added and the volatiles are removed by rotary evaporation to leave a brown oil. The oil is triturated with ether (10 ml) and the residue dried in vacuo to 84.

### 3-(4-OHPh)propanoyl-fR(NO₂)-tryptamide (85)

p-TSA.H-fR(NO₂)-tryptamide(84, 149.9 mg, 220 μmmol), 3-(4-hydroxyphenyl)propanoic acid (39.6 mg, 238 μmol), HOBt (33.7 mg, 233 μmol), NMM (0.37 ml, 337 μmol) and DMF (1.5 ml, anhydrous) are combined and cooled at 0° C. and EDCI (44.6 mg, 233 μmol) is added with stirring. After stirring at 0° C. for 1.3 h the ice bath is removed and the mixture is warmed and stirred at room temperature for 100 h. The mixture is diluted with EtOAc (20 ml) and washed with aqueous 2 N HCl (3×5 ml), the aqueous acid layer is back-extracted with EtOAc (1×10 ml), and the combined EtOAc layers are washed with 1 M NaHCO₃ (3×5 ml) and brine (10 ml). The organic extract is then dried with anhydrous Na₂SO₄, the dessicant is removed by filtration, and the filtrate is concentrated by rotary evaporation to give 85.

### 3-(2-OHPh)propanoyl-fR(NO₂)-tryptamide (86)

The procedure used in preparing compound 85 is followed to give crude coupling product from 84 (151.7 mg, 223 μmmol), 3-(2-hydroxyphenyl)propanoic acid (39.2 mg, 236 μmol), HOBt (35.3 mg, 231 μmol), NMM (0.40 ml, 364 μmol), EDCI (45.0 mg, 235 μmol), and DMF (1.5 ml, anhydrous). This material is further purified by preparative HPLC (C₄) to afford 86.

### Hydrocinnamoyl-fR(NO₂)-tryptamide (87)

The procedure used in preparing compound 85 is followed to give 87 from 84 (150.6 mg, 221 μmmol), hydrocinnamic acid (35.6 mg, 237 μmol), HOBt (34.4 mg, 225 μmol), NMM (0.37 ml, 337 μmol), EDCI (45.1 mg, 235 μmol), and DMF (1.5 ml, anhydrous).

### 3-(4-OHPh)propanoyl-fR-tryptamide (88)

The procedure used in preparing compound 32 is followed to give 88 from 95 mg (145 μmol) of 85 and 5% Pd-BaSO₄ (68 mg, unreduced).

### 3-(2-OHPh)propanoyl-fR-tryptamide (89)

The procedure used in preparing compound 32 is followed except that the product is lyophilized from acetonitrile-water rather than purified by HPLC to give 89 from 33.7 mg (51 μmol) of 86 and 5% Pd-BaSO₄ (31 mg, unreduced).

### Hydrocinnamoyl-fR-tryptamide (90)

The procedure used in preparing compound 32 is followed to give 90 from 106 mg (165 μmol) of 87 and 5% Pd-BaSO₄ (63 mg, unreduced).

### EXAMPLE 92

### Synthesis of 3-(4-OHPh)propanoyl-Me-fR-tryptamide (95)

This example is prepared according to the plan presented in Scheme XII:

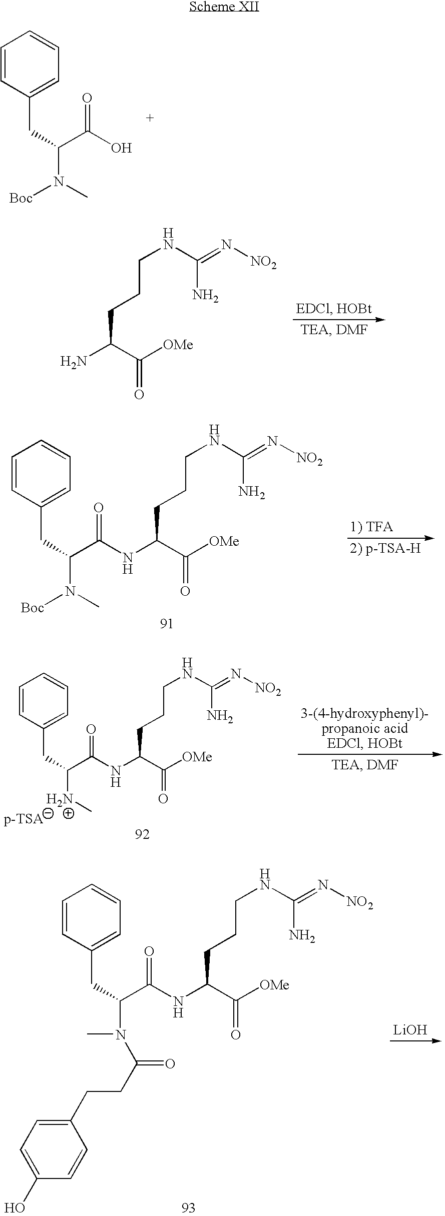

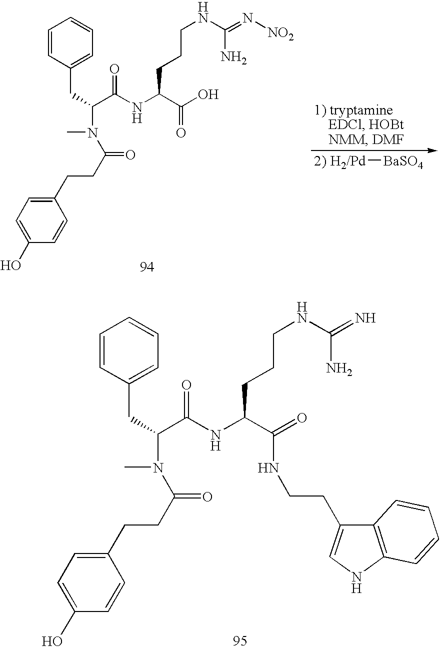

### Boc-Me-fR( NO₂)—OMe (91)

Boc-Me-f-OH (1.0008 g, 3.6 mmol), H—R(NO₂ )—OMe.HCl (0.9659 g, 3.6 mmol), HOBt (0.5515 g, 3.6 mmol), TEA (0.525 ml, 3.8 mmol) and DMF (20 ml, anhydrous) are combined and cooled at 0° C. and EDCI (0.7211 g, 3.8 mmol) is added with stirring. After stirring at 0° C. for 1 h the ice bath is removed and the mixture is warmed and stirred at room temperature for 23 h. The mixture is diluted wit h EtOAc (20 ml) and washed with aqueous 2 N HCl (3×8 ml), the aqueous acid layer is back-extracted with EtOAc (1×10 ml), and the combined EtOAc layers are washed with 1 M NaHCO₃ (3×8 ml) and brine (10 ml). The organic extract is then dried with anhydrous Na₂SO₄, the dessicant is removed by filtration, and the filtrate is concentrated by rotary evaporation to give 91.

### p-TSA.H—Me-fR(NO₂)—OMe (92)

The procedure of making compound 84 is followed to give 92 as a hydrated p-TSA salt from 91 (0.4989 g, 1.0 mmol).

### 3-(4-OHPh)propanoyl-Me-fR(NO₂)—OMe (93)

p-TSA.H—Me-fR(NO₂)—OMe (92, 282.8 mg, 500 μmmol), 3-(4-hydroxyphenyl)propanoic acid (85.7 mg, 516 μmol), HOBt (77.4 mg, 505 μmol), NMM (0.60 ml, 546 μmol) and DMF (3 ml, anhydrous) are combined and cooled at 0° C. and EDCI (101.9 mg, 532 μmol) is added with stirring. After stirring at 0° C for 1.3 h the ice bath is removed and the mixture is warmed and stirred at room temperature for 24 h. More EDCI (41.2 mg, 215 μmol) is added, the mixture is stirred at room temperature for 64 h, EDCI (29.9 mg, 156 μmol) is again added along with more NMM (0.60 ml, 546 μmol), and the mixture is heated at 50° C. for 8 h. The mixture is then left at room temperature for 21 d and is worked up as described in the procedure of preparing compound 91 to give 93.

### 3-(4-OHPh)propanoyl-Me-fR(NO₂)—OH (94)

A mixture of 3-(4-OHPh)propanoyl-Me-fR(NO₂)—OMe (93, 96.3 mg, 177 μmol), LiOH monohydrate (21.3 mg, 508 μmol) in THF-MeOH-water (6 ml, 4:1:1) is stirred at room temperature for 1 h. 6% aqueous KHSO₄ (1.3 ml) is added and the volatiles are removed by rotary evaporation. Water (1.4 ml) is added to the wet residue, the pH adjusted to ˜2 with a few more drops of 6% aqueous KHSO₄, and the aqueous mixture is extracted with EtOAc (3×2 ml). The combined organic layers are washed with water and brine and are dried over anhydrous Na₂SO₄. The spent dessicant is removed by filtration and the filtrate is concentrated by rotary evaporation to give 94.

### 3-(4-OHPh)propanoyl-Me-fR-tryptamide (95)

3-(4-OHPh)propanoyl-Me-fR(NO₂)—OH (94, 77.0 mg, 146 μmol), tryptamine (23.9 mg, 149 μmol), HOBt (25.1 mg, 164 μmol), NMM (0.17 ml, 155 μmol) and DMF (5 ml, anhydrous) are combined and cooled at 0° C. and EDCI (32.4 mg, 169 μmol) is added with stirring. After stirring at 0° C. for 1.5 h the ice bath is removed and the mixture is warmed and stirred at room temperature for 4 h. The mixture is worked up as in the procedure for preparing compound 85 to give a crude coupling product which is purified by preparative HPLC(C₄). The purified product is then dissolved in 10% acetic acid-MeOH (20 ml) and is hydrogenated under 40 psi of H₂ at room temperature for 15 h using 5% Pd—BaSO₄ (31.3 mg, unreduced) as catalyst. The catalyst is removed by filtration through Celite, the volatiles are removed in vacuo, the residue is re-dissolved in 10% acetonitrile-water (15 ml), and the mixture is frozen and lyophilized to yield 95.

### EXAMPLE 93

### Synthesis of 5-[2-Acetylamino-3-(4-chloro-phenyl)-propionylamino]-4-oxo-6-phenyl-2-(2-pyridin-2-yl-ethyl)-hexanoic acid [1-carbamoyl-2-(1H-indol-3-yl)ethyl]-amide (103)

This example is prepared according to the plans presented in Schemes XIII and XIV:

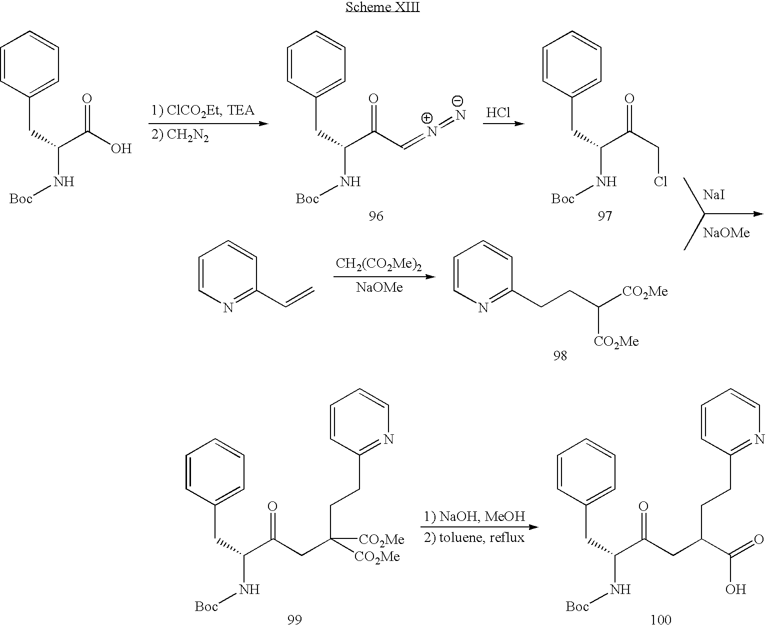

### (R)-3-(tert-Butoxycarbonylamino)-1-diazo-4-phenylbutan-2-one (96)

To a solution of Boc-f-OH (5.30 g, 20 mmol) in dry THF (100 ml) are added TEA (2.02 g, 20 mmol) and ethyl chloroformate (2.16 g, 20 mmol) at −15° C. under argon atmosphere. The mixture is stirred at −15° C. for 30 min, and is then warmed to 0° C. A solution of diazomethane in ether [100 ml, prepared from Me(NO)NCONH₂ (4.0 g, 40 mmol) and 50% aqueous KOH (20 ml)] is added. The mixture is warmed and stirred at room temperature for 3 h. The mixture is washed with saturated aqueous NaHCO₃ (30 ml), saturated aqueous ammonium chloride (30 ml), and brine (2×30 ml). The organic layer is dried over Na₂SO₄ and concentrated by rotary evaporation. The residue is crystallized from hexanes-EtOAc at 5° C. to give desired product. The mother liquor is concentrated and purified by chromatography (hexanes-EtOAc, 80:20) to give additional desired product.

### (R)-3-(tert-Butoxycarbonylamino)-1-chloro-4-phenylbutan-2-one (97)

To a solution of diazoketone (96, 115 mg, 0.4 mmol) in ether (3 ml) is added 4N HCl/1,4-dioxane (0.11 ml, 0.44 mmol, prepared by diluting conc. HCl with 1,4-dioxane) dropwise at 0° C. The resulting mixture is stirred at 0° C. for 30 min. Several drops of TEA are added to neutralize the solution and the mixture is diluted with EtOAc (20 ml), washed with saturated aqueous NaHCO₃ (10 ml), and brine (3×10 ml). The organic layer is dried over Na₂SO₄ and the solvents are evaporated. The residue is purified by chromatography (hexanes-EtOAc, 90:10) to give 108 mg (92%) of a colorless solid: m.p.: 101-102° C.; ¹H NMR (CDCl₃, 400 MHz) δ 1.43(s, 9H), 3.03, 3.10 (ABX, J_{AB}=13.8 Hz, J_{AX}=7.1 Hz, J_{BX}=6.8 Hz, 2H), 4.00, 4.19 (AB, J_{AB}=16.2 Hz, 2H), 4.70 (ml, 1H), 5.04 (br d, J=6.8 Hz, 1H), 7.18-7.37 (m, 5H).

### Methyl 2-methoxycarbonyl-4-(2′-pyridinyl)butanoate (98)

To freshly prepared NaOMe [from 2.3 g (0.1 mol) of sodium] in MeOH (25 ml) is added dimethyl malonate (32 g, 0.24 mmol). A solution of 2-vinylpyridine (10.5 g, 0.1 mol) in MeOH (15 ml) is added dropwise over 40 min to the NaOMe solution at reflux. The resulting mixture is heated at reflux for 2.5 h. MeOH is removed under reduced pressure, the residue is treated with 2N HCl (150 ml), then is extracted with ether (2×60 ml) to remove excess dimethyl malonate. The aqueous phase is made basic with 2N NaOH and extracted with ether (3×100 ml). The combined organic phases are washed with brine (3×60 ml) and dried over Na₂SO₄. The filtrate is concentrated under reduced pressure, then most of the excess 2-vinylpyridine is removed in vacuo. The residue is purified by chromatography (hexanes-EtOAc, 60:40 to 50:50 to 30:70) to give desired product.

### Methyl (R)-5-(tert-Butoxycarbonylamino)-2-methoxycarbonyl-6-phenyl-2-[2′-(2″-pyridinyl)ethyl]-4-oxohexanoate (99)

To a solution of the α-chloroketone (97, 150 mg, 0.5 mmol) in dry 1,2-dimethoxyethane (3.0 ml) is added Nal (75 mg, 0.5 mmol) and the mixture is stirred under an argon atmosphere for 15 min (Mixture A). To a solution of the diester (98, 142 mg, 0.6 mmol) in dry 1,2-dimethoxyethane (3.0 ml) is added freshly prepared NaOMe (32 mg, 0.6 mmol) and the mixture is stirred under an argon atmosphere for 15 min (Mixture B). Mixture B is added to mixture A and the resulting mixture is stirred at room temperature for 1 h. The mixture is diluted with EtOAc (30 ml) and washed with brine (2×10 ml). The organic layer is dried over Na₂SO₄, the solvents are removed by rotary evaporation, and the residue is purified by chromatography (hexanes-¹PrOH, 80:20) to give 99.

### (2RS, 5R)-5-(tert-Butoxycarbonylamino)-6-phenyl-2-[2′-(2″-pyridinyl)ethyl]-4-oxohexanoic acid (100)

To a solution of di-ester (99, 165 mg) in MeOH (10 ml) is added 5N NaOH (1.0 ml) and the resulting mixture is stirred at room temperature for 2 h. The mixture is concentrated by rotary evaporation and the residue is dissolved in water (10 ml) and acidified with 3N HCl to pH=3. The mixture is extracted with DCM (3×10 ml) and the combined organic phases are washed with brine (10 ml) and dried over Na₂SO₄. The solvents are removed by rotary evaporation to give the di-acid. The crude di-acid is suspended in toluene (10 ml) and the mixture is heated at reflux under an argon atmosphere for 3 h. The solvent is evaporated and the residue is purified by chromatography (DCM-MeOH, 98:2 to 95:5) to give a mixture of two diastereoisomers that are separable by preparative HPLC.

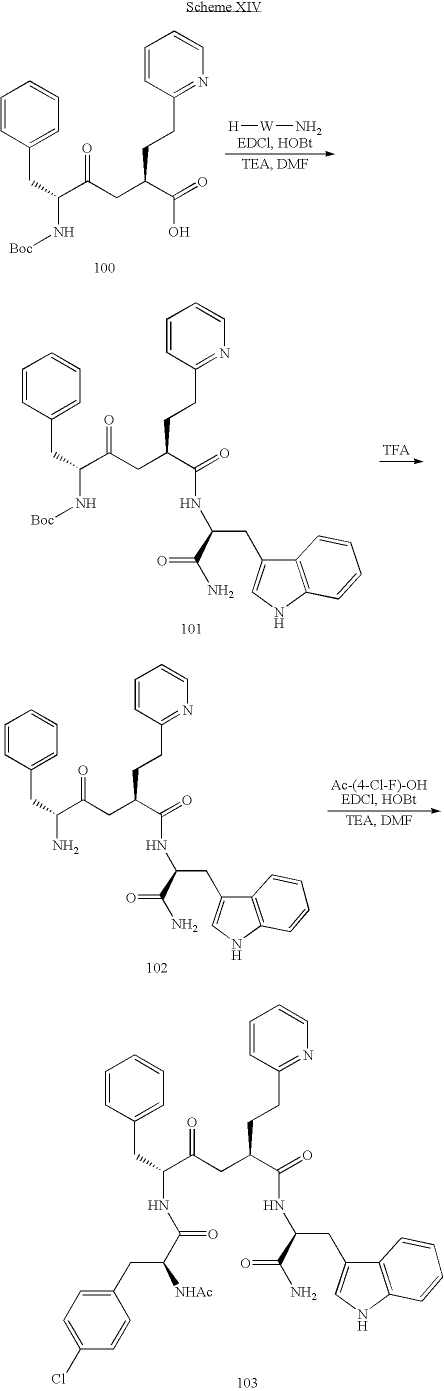

### {1-Benzyl-4-[1-carbamoyl-2-(1H-indol-3-yl)-ethylcarbamoyl]-2-oxo-6-pyridin-2-yl-hexyl}-carbamic acid tert-butyl ester (101)

Acid 100 (44 mg, 0.1 mmol), H—W—NH₂.HCl (26 mg, 0.11 mmol), HOBt(16 mg, 0.12 mmol), TEA (24 mg, 0.24 mmol), EDCI (24 mg, 0.12 mmol) and DMF(1 ml, anhydrous) are combined. The mixture is stirred at room temperature overnight, then poured into water (10 ml) and extracted with EtOAc (4×9 ml). The combined extract is washed with 1 N HCl (2×8 ml), saturated NaHCO₃ (1×8 ml), brine (8 ml) then dried with anhydrous Na2SO₄. After filtration, the filtrate is concentrated by rotary evaporation to give 101.

### 5-Amino-4-oxo-6-phenyl-2-(2-pyridin-2-yl-ethyl)hexanoic acid [1-carbamoyl-2-(1H-indol-3-yl)ethyl]-amide (102)

Peptide 101 (97 mg, 0.16 mmol) is mixed with a solution (1 ml) of anisole-TFA-DCM (1:8:9). The mixture is stirred at room temperature overnight. After removal of solvent, the resulting residue is co-evaporated with ether (2×8 ml) to give 0.81 g (99%) of 102.

### Ac-(4-Cl—F)—OH

A mixture of Boc-(4-Cl—F)—OH (54 mg, 0.18 mmol), DCM (0.5 ml), and TFA (0.5 ml) is stirred at room temperature for 2 h. After removal of solvent, the resulting residue is mixed with DCM (1 ml), acetic anhydride (17 mg, 0.2 mmol), and TEA (40 mg, 0.4 mmol). The mixture is stirred at room temperature overnight, then concentrated by rotary evaporation to yield Ac-(4-Cl—F)—OH.

### 5-[2-Acetylamino-3-(4-chloro-phenyl)-propionylamino]-4-oxo-6-phenyl-2-(2-pyridin-2-yl-ethyl)-hexanoic acid [1-carbamoyl-2-(1H-indol-3-yl)ethyl]-amide (103)

EDCI (36 mg, 0.19 mmol) is added to a mixture of peptide 102 (81 mg, 16 mmol), Ac-(4-Cl—F)—OH (41 mg, 0.17 mmol), HOBt (24 mg, 0.18 mmol), TEA (35 mg, 0.35 mmol), and DMF (2 ml, anhydrous) at 0° C. The reaction mixture is stirred at room temperature overnight and then is worked up as in the procedure for making compound 101. The crude product is purified by preparative HPLC to give 103.

### EXAMPLE 94

### Synthesis of N-[3-[9-Benzyl-12-(4-chlorobenzyl)-3-(1H-indol-3ylmethyl-2,5,8,14-tetraoxo-1,4,7,13-tetraaza-cyclotetracos-6-yl]-propyl}-guanidine (110)

This example is prepared according to the plan presented in Scheme XV:

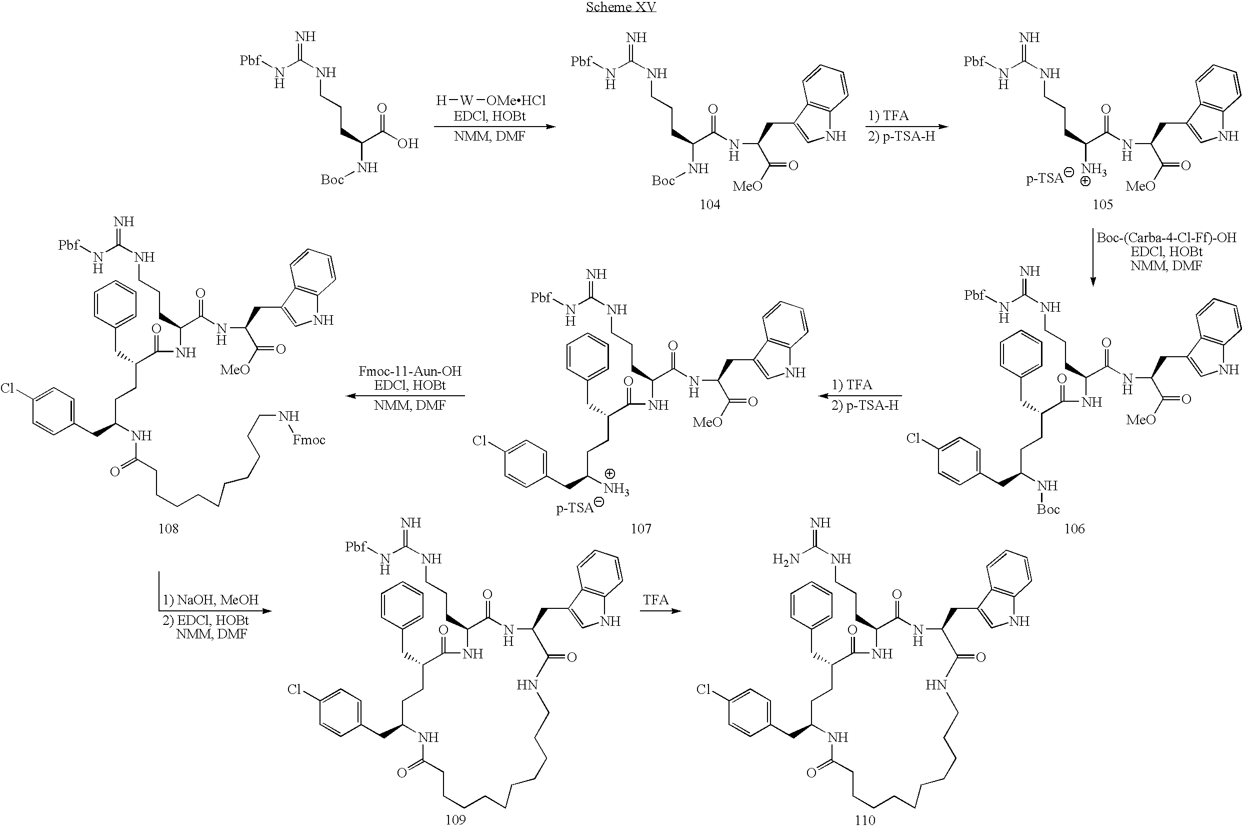

### Boc-R(Pbf)W—OMe (104)

NMM (0.48 ml, 4.4 mmol) is added to a mixture of Boc-R(Pbf)-OH (1.053 g, 2.0 mmol), H—W—OMe (HCl(0.509 g, 2.0 mmol), HOBt (0.270 g, 2.0 mmol), EDCI (0.422 g, 2.2 mmol) in DMF (10 mmol) at 0° C. and the resulting mixture is stirred at 0° C. for 1 h and then at room temperature for 4 h. The reaction mixture is diluted with EtOAc (100 ml) and is washed successively with water (2×10 ml), 1 N HCl (2×10 ml), saturated NaHCO₃, 2×10 ml), and brine (2×10 ml). The organic layer is dried over MgSO₄, the dessicant removed by filtration, and the filtrate concentrated by rotary evaporation to yield 1.454 g(100%) of crude 104.

### p-TSA.H—R(Pbf)W—OMe (105)

A solution of 104 (1.454, 2.0 mmol) in a mixture of TFA-DCM-water (10:40:0.5, 20 ml) and DCM (20 ml) is stirred at room temperature for 6 h. p-Toluenesulf-onic acid monohydrate (0.380 g, 2.0 mmol) is added and the mixture stirred for 10 min at room temperature. The solvent is removed by rotary evaporation and the residue is triturated with ether-hexane (1:1, 100 ml) to yield 1.598 g (99.9%) of crude 105.

### Boc-(carba-4-Cl—Ff)-R(Pbf)W—OMe (106)

EDCI (105.5 mg, 0.55 mmol) and NMM (0.12 ml, 1.1 mmol) are added to a well-stirred mixture of 5 (216 mg, 0.5 mmol), 105 (399.5 mg, 0.5 mmol), and HOBt (67.6 mg, 0.5 mmol) in DMF (9 ml) at 0° C. The resulting mixture is stirred at 0° C. for 1 h and then at room temperature for 5 h. The mixture is worked-up as in the procedure for preparing compound 104 to give 508 mg (98%) of 106.

### p-TSA.H-(carba-4-Cl—Ff)-R(Pbf)W—OMe (107)

A solution of 106 (508 mg, 488 μmol) in TFA-DCM-water (10:90:0.5, 15 ml) is stirred at room temperature for 17 h. Analysis by HPLC indicates that the reaction is not yet complete so more TFA-DCM-water (10:90:0.5, 10 ml) and water (1 ml) are added and the resulting mixture is stirred at room temperature for 7 d. p-Tolu-enesulfonic acid monohydrate (92.8 mg, 488 μmol) is added and the mixture stirred for 10 min at room temperature. The solvent is removed by rotary evaporation and the residue is triturated with ether (25 ml) to yield crude 107.

### Fmoc-11-Aun-(carba-4-Cl—Ff)-R(Pbf)W—OMe (108)

The procedure of making compound 104 is followed and crude 108 is obtained from Fmoc-11-Aun-OH (207 mg, 488 μmol), 107 (543 mg, 488 μmol), HOBt (66.0 mg, 488 μmol), EDCI (103.0 mg, 537 μmol), and NMM (0.118 ml, 1.07 mmol).

### [11-Aun-(carba-4-Cl—Ff)-R(Pbf)W] (109)

1 N NaOH (2.2 ml, 2.2 mmol) is added to a solution of 108 (520 mg, 387 μmol) in THF-MeOH (1:1) at room temperature and the resulting mixture is stirred at room temperature for 2 h. The mixture is acidified with 1 N HCl to pH 3 and is partitioned between EtOAc (100 ml) and water (20 ml). The water phase is further extracted with EtOAc (2×20 ml) and the combined organic phase is washed with brine (20 ml) and dried over MgSO₄. The dessicant is removed by filtration, the filtrate concentrated by rotary evaporation, and the residue triturated with ether (20 ml) to the crude amino acid to be cyclized. NMM (47 μl, 460 μmol) is added to a mixture of this crude amino acid (430 mg, 387 μmol), HOBt (53 mg, 387 μmol), and EDCI (82.5 mg, 430 μmol), in DMF at 0° C. The resulting mixture is stirred at 0° C. for 1 h and then at room temperature for 14 h. The mixture is worked-up as in the procedure for preparing compound 104 and purified by preparative HPLC to give 109.

### [11-Aun-(carba-4-Cl—Ff)-RW] (110)

A mixture of 109 (42 mg, 39 μmol) in TFA-DCM-water (10:10:0.5, 5 ml) is stirred at room temperature for 20 h, the volatiles are removed by rotary evaporation, and the residue is purified by preparative HPLC. The product-containing fractions are combined, concentrated, frozen, and lyophilized to yield 110 as a TFA salt.

### EXAMPLES 95-99

Coupling procedure (CP) for peptide bond formation in solution:

An amine component (1 equivalent), an acid component (1 equivalent), and HOBt (2 equivalents) are dissolved in DMF (2 ml/mmole of substrate). The solution is treated with N-methylmorpholine (3-4 equivalents), EDCI (1.2 equivalent) and stirred at room temperature until formation of the product is complete (usually 1-5 hours). The product precipitates upon addition of water (6-10 ml/ml DMF) to the reaction mixture and is separated from the liquid by filtration or decantation.

### A. Synthesis of core dipeptide Boc-DPhe-Arg(NO₂)OH (A-2)

### A-1. Boc-DPhe-Arg(NO₂)OMe

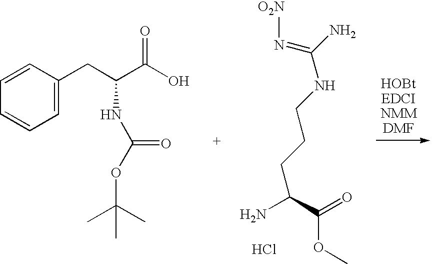

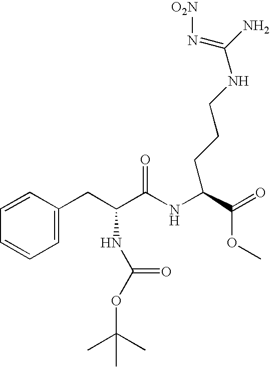

| Reactant/Reagent: | MW: | Moles: | Amount: | Units: |
|---|---|---|---|---|
| Boc-D-Phe | 265.31 | 0.05 | 13.26 | g |
| DMF | | | 50 | ml |
| EDCI | 191.17 | 0.06 | 11.5 | g |
| H-Arg(OMe)HCl | 269.69 | 0.05 | 13.48 | g |
| HOBt | 135.13 | 0.1 | 13.5 | g |
| NMM | 101.14 | 0.15 | 16.5 | ml |

The coupling procedure (CP) for the peptide bond formation in solution is used.

### A-2. Boc-DPhe-Arg(NO₂)OH

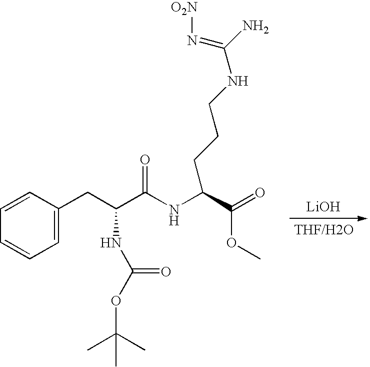

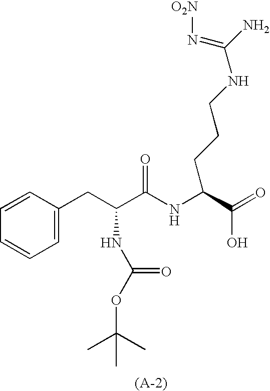

| Reactant/Reagent: | MW: | Moles: | Amount: | Units: |
|---|---|---|---|---|
| Boc-DPhe-Arg(NO₂)OMe | 480.23 | 0.01 | 4.8 | g |
| LiOH | 23.95 | 0.023 | 0.55 | g |
| THF | | | 20 | ml |
| water | | | 10 | ml |

A solution of Boc-DPhe-Arg(NO2)OMe in THF is cooled in an ice bath and treated at 0° C. with aqueous solution of LiOH. The reaction mixture is stirred in the ice bath for 4 hrs. Solvents are evaporated to small residual volume, which is treated with 1N HCl (approx 25 ml) to pH 2-3. The product is extracted with ethyl acetate, washed with water/brine, dried with anhydrous magnesium sulfate; the solvent is evaporated to dryness to afford the title compound.

### B. Synthesis of Amino Terminus Groups

### B-1. 3-(4-Benzyloxy-phenyl)-propionic acid

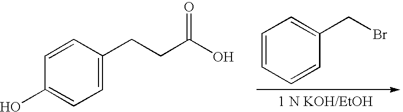

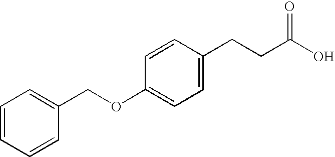

| Reactant/Reagent: | MW: | Moles: | Amount: | Units: |
|---|---|---|---|---|
| n-hydroxypropionic acid | 166.17 | 0.0468 | 7.78 | g |
| benzyl bromide | 171.03 | 0.048 | 8.17 | g |
| NaOH 1N | | | 100 | ml |
| EtOH | | | 150 | ml |

The procedure described in JACS 1955, 77, p. 4887-4892 is used. The product precipitates when the reaction mixture is acidified to pH 2-3.

### B-2. 3-(4-Benzyloxy-phenyl)-propionyl chloride

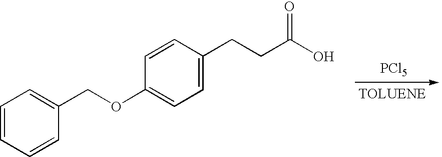

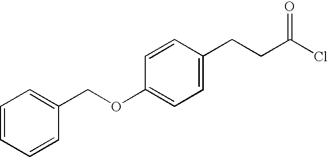

| Reactant/Reagent: | MW: | Moles: | Amount: | Units: |
|---|---|---|---|---|
| 3-(4-Benzyloxy-phenyl)- | 256.11 | 0.038 | 9.73 | g |
| propionic acid | | | | |
| PCl₅ 95% | 208.24 | 0.0418 | 9.15 | g |
| toluene | | | 400 | ml |

Solid PCl₅ is added to a solution of 3-(4-benzyloxy-phenyl)-propionic acid in toluene over a 1 hour period. The reaction mixture is stirred at room temperature for 3 hours and solvent is evaporated. The residue is stirred with hexanes overnight producing crystalline material, which is filtered and dried under vacuum.

### B-3. 4-(S)-Benzyl-3-[3-(4-benzyloxy-phenyl)-propionyl]-oxazolidin-2-one

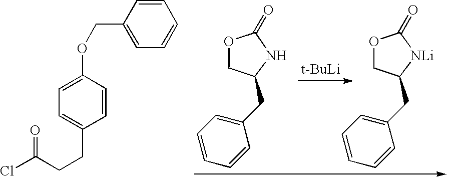

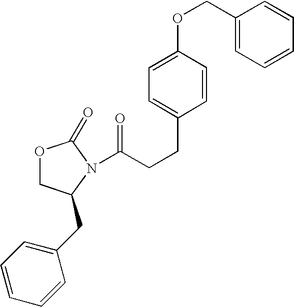

| Reactant/Reagent: | MW: | Moles: | Amount: | Units: |
|---|---|---|---|---|
| benzyloxyphenylpropanoyl | 274.74 | 0.005 | 1.37 | g |
| chloride | | | | |
| (S)-(−)-4-benzyloxazolidinone | 177.2 | 0.005 | 0.89 | g |
| tert-BuLi 1.7 in pentane | | 0.0051 | 3 | ml |
| THF dry | | | 6 | ml |
| THF dry | | | 6 | ml |

The procedure described in Tetrahedron 52(43), 1996, p 13733-13738 is used. Li—(S)-(−)-4-benzyloxazolidinone salt is prepared at −65° to −72° C. A solution of decanoyl chloride in THF is cooled to −72° C. and treated with Li—(S)-(−)-4-benzyloxazolidinone solution at this temperature. The reaction mixture is stirred at −70° to −75° C. for 1 hour and overnight at room temperature, treated with NH₄Cl solution and extracted with ethyl acetate. The organic layer is washed with water/brine, dried with MgSO₄, and evaporated. The residue is separated on a silica column using hexane/ethyl acetate 7/3 solution to afford the title compound.

### B-4. 4-(S)-Benzyl-3-[2-(4-benzyloxy-benzyl)-pent-4-enoyl]-oxazolidin-2-one

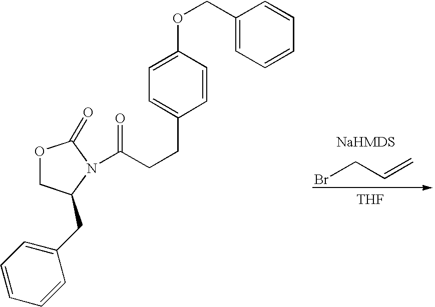

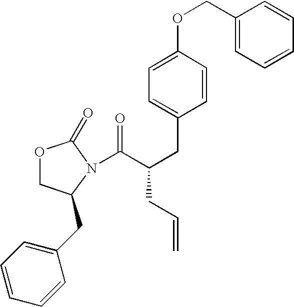

| Reactant/Reagent: | MW: | Moles: | Amount: | Units: |
|---|---|---|---|---|
| 4-(S)-Benzyl-3-[3-(4-benzyloxy- | 415.48 | 0.003 | 1.24 | g |
| phenyl)-propionyl]-oxazolidin-2- | | | | |
| one | | | | |
| allyl bromide, d=1.398 | 120.98 | 0.006 | 0.52 | ml |
| THF | | | 30 | ml |
| NaHMDS, 0.6 M in THF | | 0.003 | 5 | ml |

NaHMDS is added to a THF solution of 4-(S)-benzyl-3-[3-(4-benzyloxy-phenyl)-propionyl]-oxazolidin-2-one at −70° to −75° C. over 15 min. The mixture is stirred for 1 hour and treated with allyl bromide at −70° C. Stirring is continued at this temperature for 1 hour. The reaction mixture is allowed to reach 0° C. in 3 hours and quenched with 10% NH₄Cl. The product is extracted with ethyl acetate, washed with water/brine and dried with anhydrous magnesium sulfate. The solvent is evaporated and the crude product is purified on a silica column using hexane 4/ethyl acetate 1 to afford the title compound.

### B-5. 2-(4-Benzyloxy-benzyl)-pent-4-enoic acid

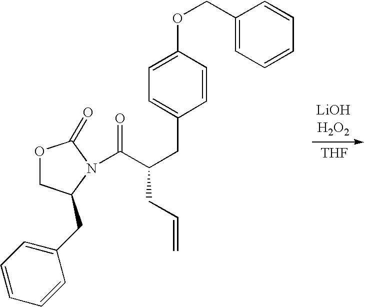

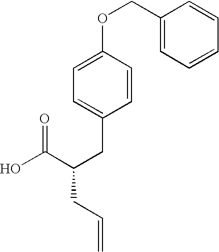

| Reactant/Reagent: | MW: | Moles: | Amount: | Units: |
|---|---|---|---|---|
| amide | 455.54 | 0.004 | 1.85 | g |
| H₂O₂ 30% | | 0.016 | 1.82 | ml |
| LiOH | 23.95 | 0.008 | 0.19 | g |
| THF | | | 10 | ml |

The procedure described in JOC 1992, 57(10), 2888-2902 (p.2894) is used.

### B-6. 4-(4-(S)-Benzyl-2-oxo-oxazolidin-3-y)-3-(4-benzyloxy-benzyl)-4-oxo-butyronitrile

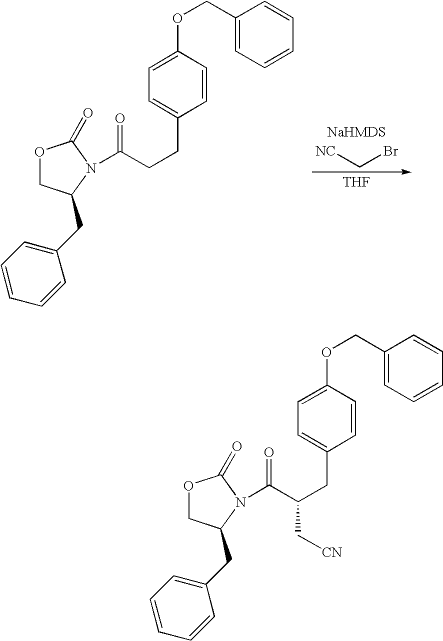

A procedure analogous to that used to prepare B-4 is used to prepare B-6.

### B-7. 4-Amino-2-(4-hydroxy-benzyl)-butyric acid ethyl ester; hydrochloride

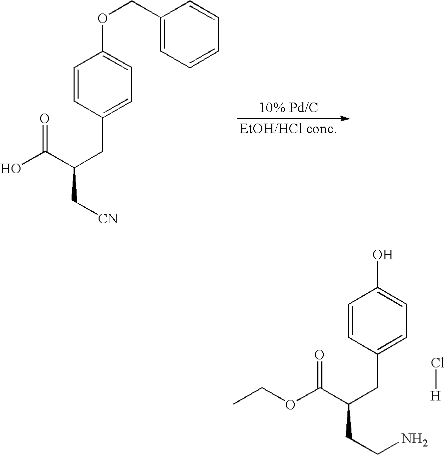

2-Cyanomethyl-3-phenyl-propionic acid (8.16 g, 43 mmol) in a solution of ethanol (75 ml) and concentrated HCl (10 ml) is hydrogenated overnight at 40 psi in the presence of 10% Pd/C. The catalyst is removed by filtration; the filtrate is concentrated under reduced pressure to dryness to afford the title compound.

### B-8. 3-(4-Benzyloxy-phenyl)-2-decanoylamino-propionic acid methyl ester

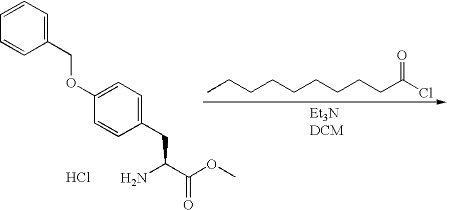

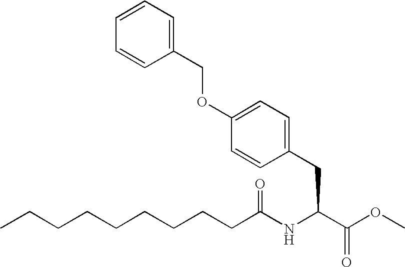

| Reactant/Reagent: | MW: | Moles: | Amount: | Units: |
|---|---|---|---|---|
| H-Tyr(Bzl)-OMe HCl | 321.8 | 0.003 | 0.965 | g |
| decanoyl chloride 98% d=0.919 | 190.71 | 0.006 | 1.27 | ml |
| TEA d=0.726 | 101.19 | 0.016 | 2.2 | ml |
| DCM | | | 15 | ml |

TEA is added to a solution of the remaining reactants in DCM at −2° to +3° C. The reaction mixture is stirred at room temperature for 4 hrs and diluted with 0.1N HCl. The product is extracted with DCM, washed with water, dried with MgSO₄, and solvent is evaporated under reduced pressure. The residue is crystallized from hexanes to afford the title compound.

### B-9. 3-(4-Benzyloxy-phenyl)-2-decanoylamino-propionic acid

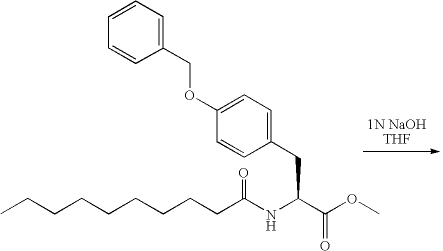

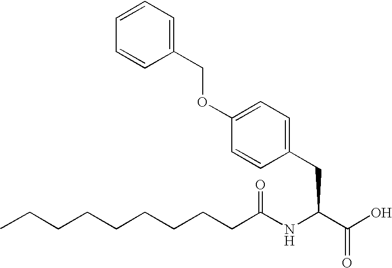

| Reactant/Reagent: | MW: | Moles: | Amount: | Units: |
|---|---|---|---|---|
| Ester | 439.59 | 0.00223 | 0.98 | g |
| 1 N NaOH | | 0.0023 | 2.3 | ml |
| THF | | | 4.3 | ml |
| water | | | 0.7 | ml |

Reactants are stirred at room temperature for 5 hrs. Solvents are evaporated under reduced pressure; the residue is diluted with water and acidified to pH about 2. Resulting precipitate of the product is filtered, washed with water until pH of the filtrate reaches about 6, and dried under vacuum overnight.

### C. Synthesis of Carboxyl Terminal Groups

### C-1. N¹-Benzyl-3-(1H-indol-3-yl)-N¹-methyl-propane-1,2-diamine

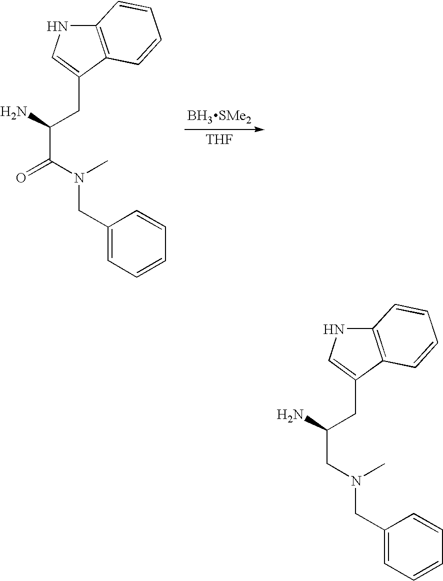

A 2 M THF solution of BH₃ Me₂S complex (40 ml) is added to a solution of the amide substrate (4.5 g, 8.4 mmol) in anhydrous THF (50 ml). The reaction mixture is heated at 75° C. while slowly distilling liquid is collected through the condenser. After 2 hours, a new portion of 2 M THF solution of BH₃ Me₂S complex (10 ml) is added and heating with simultaneous distillation is continued for additional 3 hours. The reaction mixture is cooled to room temperature and carefully treated with MeOH, until the release of gas ceases, and with 3 N NaOH. The crude product is extracted with ethyl acetate and purified on a silica column using a solution of 1.5% MeOH in AcOEt following by a solution of EtOAc/DCM/MeOH/Et₃N 4/5/0.5/0.3 to afford the title compound.

### C-2. N¹-Benzyl-N¹-hexyl-3-(1H-indol-3-yl)-propane-1,2-diamine

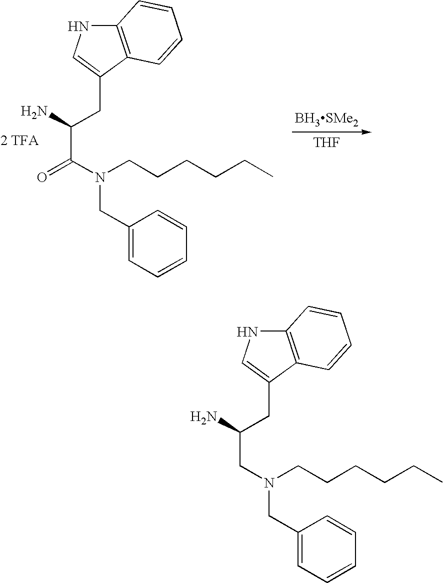

A procedure analogous to that used to prepare C-1 was used to prepare C-2.

### C-3. N¹-Hexyl-3-(1H-indol-3-yl)-N¹-methyl-propane-1,2-diamine

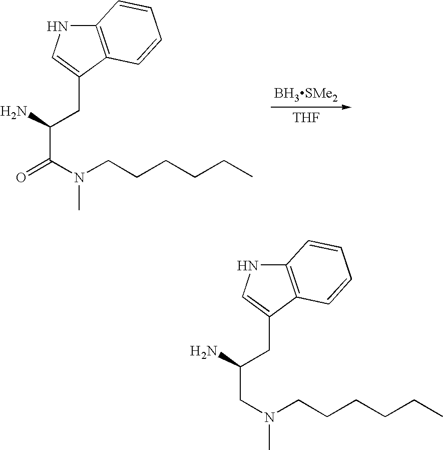

A procedure analogous to that used to prepare C-1 was used to prepare C-3.

### D. Assembly of Tetrapeptide Mimetics

### D-1. (1-{4-Nitroguanidino-1-[2-(hexyl-methyl-amino)-1-(1H-indol-3-ylmethyl)-ethylcarbamoyl]-butylcarbamoyl]-2-phenyl-ethyl)-carbamic acid tert-butyl ester

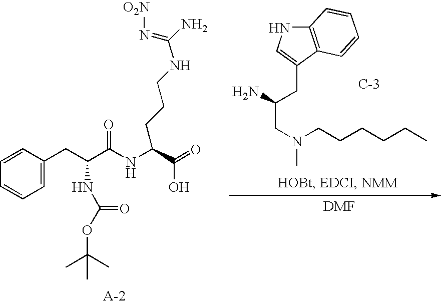

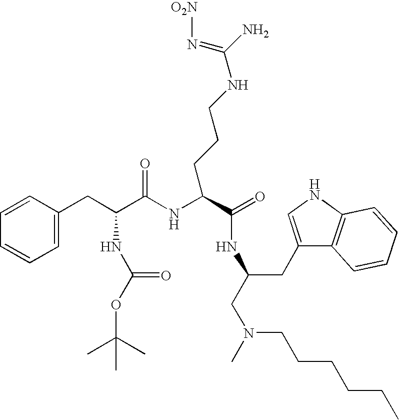

| Reactant/Reagent: | MW: | Moles: | Amount: | Units: |
|---|---|---|---|---|
| dipeptide | 466.49 | 0.0005 | 0.233 | g |
| N¹-Hexyl-3-(1H-indol-3-yl)-N¹- | 287.24 | 0.0005 | 0.143 | g |
| methyl-propane-1,2-diamine | | | | |
| HOBt | 135.12 | 0.001 | 0.135 | g |
| NMM d=0.92 | 101.14 | 0.0015 | 0.17 | ml |
| EDCI | 191.17 | 0.0006 | 0.114 | g |
| DMF | | | 1 | ml |

The coupling procedure (CP) for the peptide bond formation in solution is used. The crude product is purified on a silica column using hexane/ethyl acetate6/1 to afford the title compound.

A procedure analogous to that used to prepare D-1 is used to prepare the following D-2, D-3, D-4 and D-5.

### D-2. 2-[2-(2-tert-Butoxycarbonylamino-3-phenyl-propionylamino)-5-nitroguanidino-pentanoylamino]-3-(1H-indol-3-yl)-propionic acid methyl ester

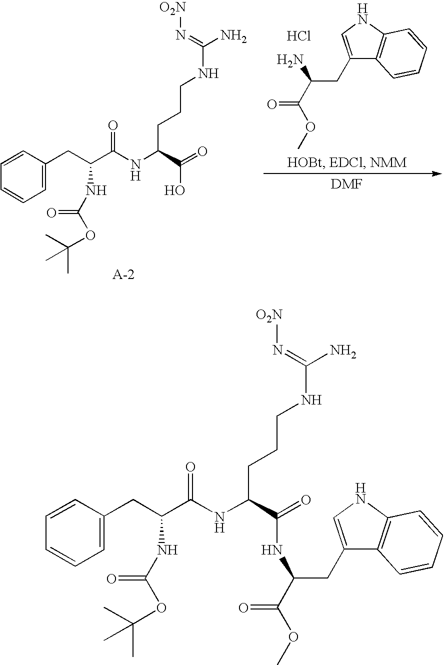

### D-3. ([-1[-2-(Benzyl-hexyl-amino)-1-(1H-indol-3-ylmethyl)-ethylcarbamoyl]-4-nitroguanidino-butylcarbamoyl}-2-phenyl-ethyl)-carbamic acid tert-butyl ester

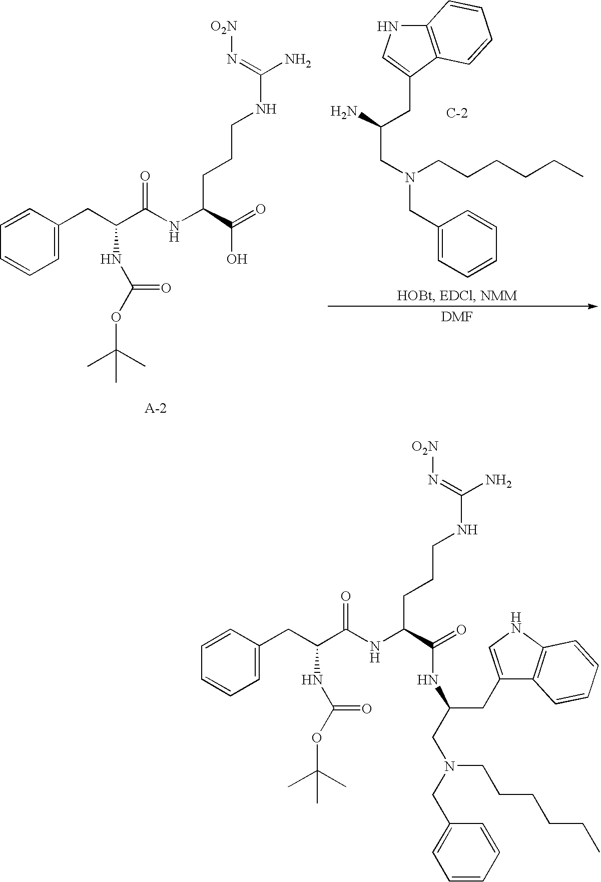

### D-4. (1-{4-Nitroguanidino-1-[2-(1H-indol-3-yl)-1-(methyl-propyl-carbamoyl)-ethylcarbamoyl]-butylcarbamoyl}-2-phenyl-ethyl)-carbamic acid tert-butyl ester

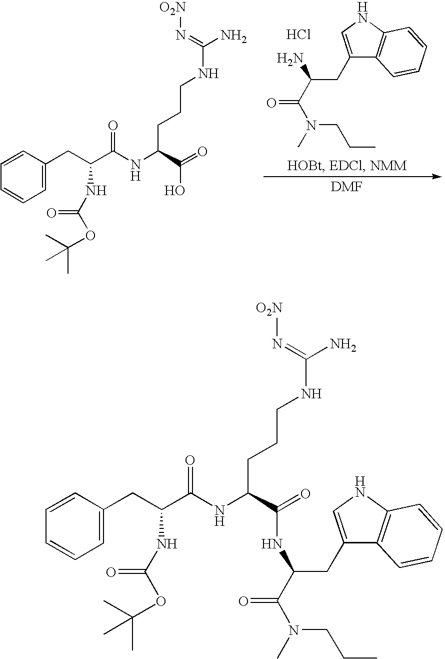

### D-5. (1-{1-[1-(Carbamoylmethyl-methyl-carbamoyl)-2-(1H-indol-3-yl)-ethylcarbamoyl]-4-nitroguanidino-butylcarbamoyl}-2-phenyl-ethyl)-carbamic acid tert-butyl ester

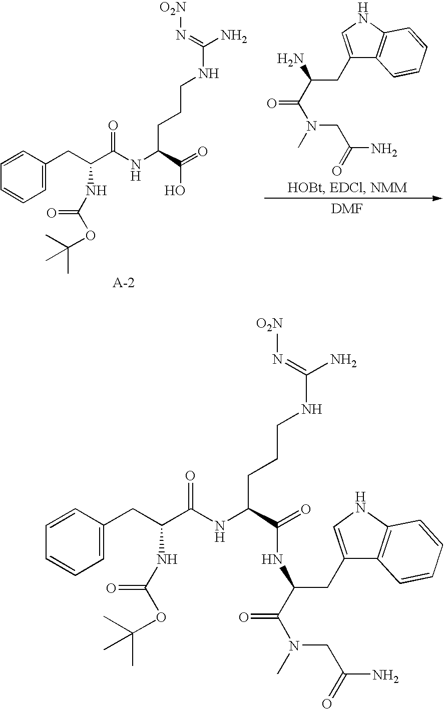

### D-6. 2-(2-Amino-3-phenyl-propionylamino)-5-nitroguanidino-pentanoic acid [2-(hexyl-methyl-amino)-1-(1H-indol-3-ylmethyl)-ethyl]-amide

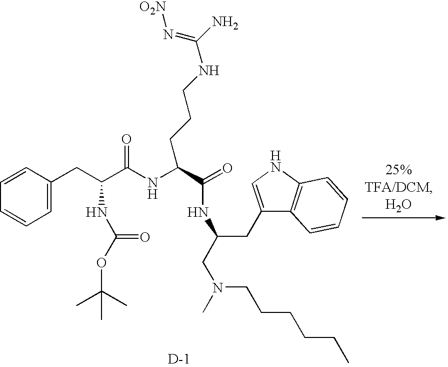

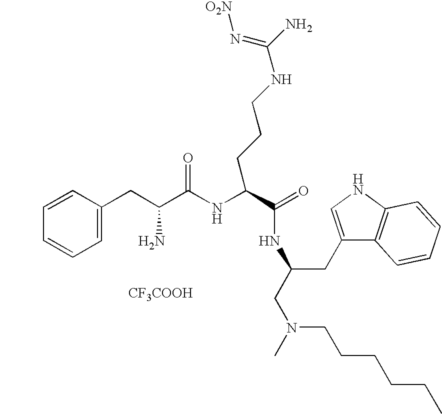

| Reactant/Reagent: | MW: | Moles: | Amount: | Units: |
|---|---|---|---|---|
| tripeptide | | | 0.15 | g |
| TFA/DCM/H2O 1/2/0.1 | | 2 | ml | |

The reaction mixture is stirred at room temperature for 4 hours and diluted with 1,2-dichloethane. Solvents are evaporated under reduced pressure; the residue is dried under vacuum overnight.

### D-7. 2-(4-Benzyloxy-benzyl)-pent-4-enoic acid (1-{4-nitroguanidino-1-[2-(hexyl-methyl-amino)-1-(1H-indol-3-ylmethyl)-ethylcarbamoyl]-butylcarbamoyl}-2-phenyl-ethyl)-amide

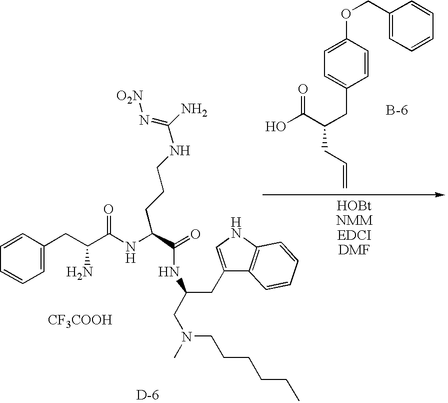

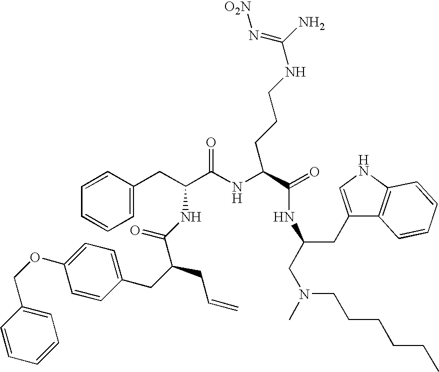

| Reactant/Reagent: | MW: | Moles: | Amount: | Units: |
|---|---|---|---|---|
| Amide bond formation | 749.82 | 0.000226 | 0.17 | g |
| acid | 296.36 | 0.0003 | 0.09 | g |
| HOBt | 135.12 | 0.0006 | 0.08 | g |
| NMM d=0.92 | 101.14 | 0.001 | 0.11 | ml |
| EDCI | 119.17 | 0.00036 | 0.069 | g |
| DMF | | | 0.7 | ml |

The coupling procedure (CP) for the peptide bond formation in solution is used. The crude product is purified by by reverse phase preparative HPLC to afford the title compound.

### EXAMPLE 95

### Synthesis of 2-(4-Hydroxy-benzyl)-pentanoic acid (1-{4-guanidino-1-[2-(hexyl-methyl-amino)-1-(1H-indol-3-ylmethyl)-ethylcarbamoyl]-butylcarbamoyl}-2-phenyl-ethyl)-amide

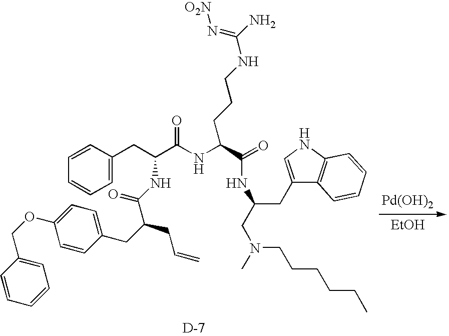

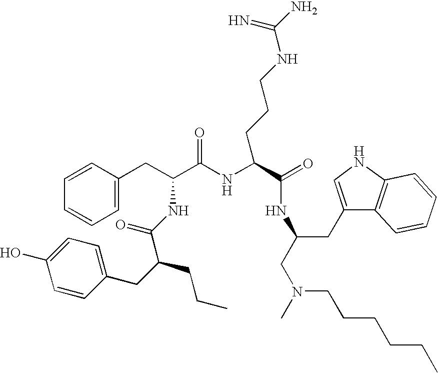

| Reactant/Reagent: | MW: | Moles: | Amount: | Units: |
|---|---|---|---|---|
| Starting material (D-7) | 913.15 | | 0.1 | g |
| EtOH | | | 15 | ml |
| Pd(OH)2 | | | | |

The reaction mixture is hydrogenated at room temperature, 45 psi overnight. The catalyst is separated by filtration through Celite. Solvent is evaporated under reduced pressure. The crude product is purified by reverse phase preparative HPLC to afford the title compound.

### EXAMPLE 96

### Synthesis of Decanoic acid [1-(1-{4-guanidino-1-[2-(hexyl-methyl-amino)-1-(1H-indol-3-ylmethyl)-ethylcarbamoyl]-butylcarbamoyl}-2-phenyl-ethylcarbamoyl)-2-(4-hydroxy-phenyl)-ethyl]-amide

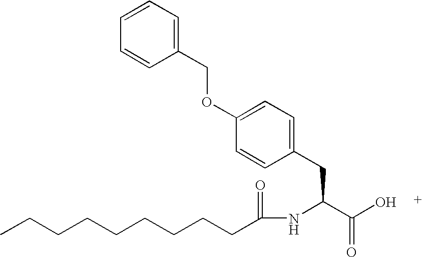

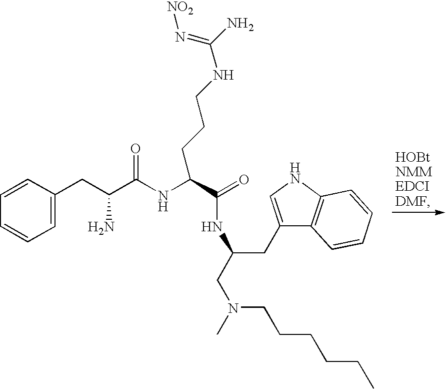

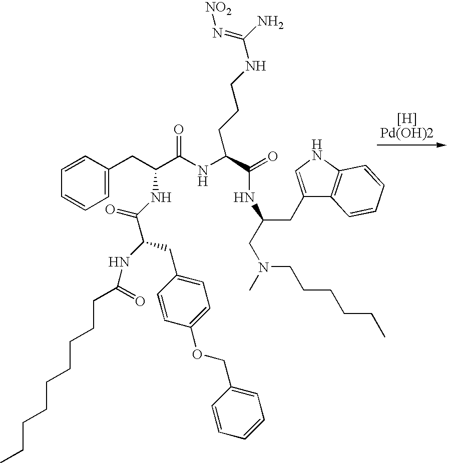

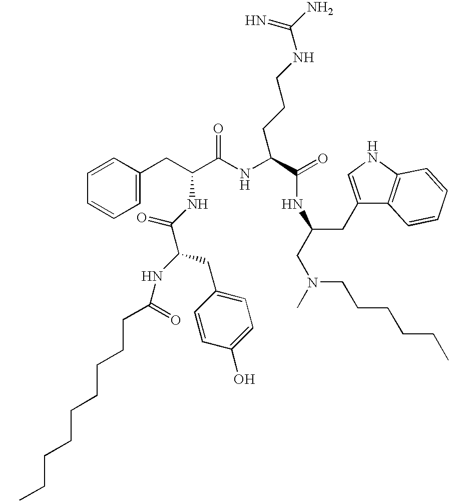

| Reactant/Reagent: | MW: | Moles: | Amount: | Units: |
|---|---|---|---|---|
| acid | 426.56 | 0.00023 | 0.1 | g |
| amine | 635.8 | 0.00019 | 0.12 | g |
| HOBt | 135.12 | 0.0005 | 0.07 | g |
| NMM d=0.92 | 101.14 | 0.001 | 0.11 | ml |
| EDCI | 191.17 | 0.0003 | 0.06 | g |
| DMF | | | 1 | ml |
| Pd(OH)2 | | | | |

The coupling procedure (CP) for the peptide bond formation in solution is used. Hydrogenation is performed in ethanol at 45 psi for 48 hrs. The catalyst is removed by filtration; the crude product is purified by reverse phase preparative HPLC to afford the title compound.

A procedure analogous to that used to make Example 95 was used to prepare the compound Of Examples 97-99.

### EXAMPLE 97

### Synthesis of 2-(5-Guanidino-2-{2-[2-(4-hydroxy-benzyl)-pentanoylamino]-3-phenyl-propionylamino}-pentanoylamino)-3-(1H-indol-3-yl)-propionic acid methyl ester

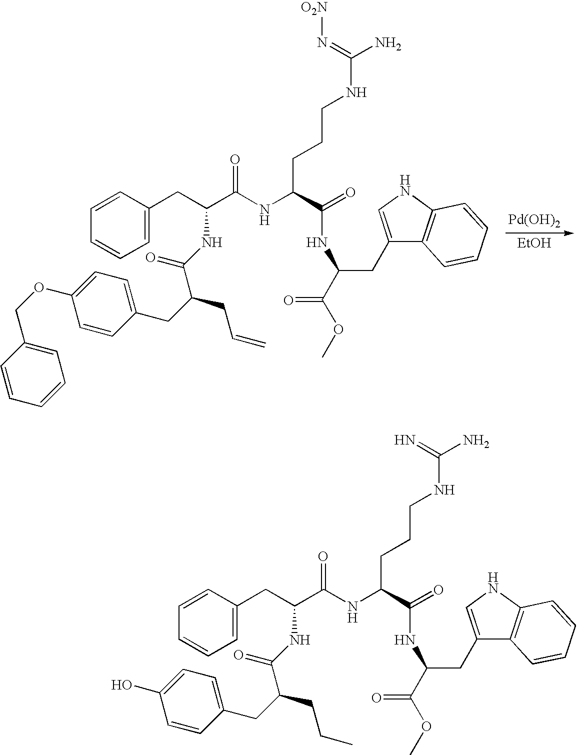

### EXAMPLE 98

### Synthesis of 2-(4-Hydroxy-benzyl)-pentanoic acid (1-{1-[1-(benzyl-methyl-carbamoyl)-2-(1H-indol-3-yl)-ethylcarbamoyl]-4-guanidino-butylcarbamoyl}-2-phenyl-ethyl)-amide

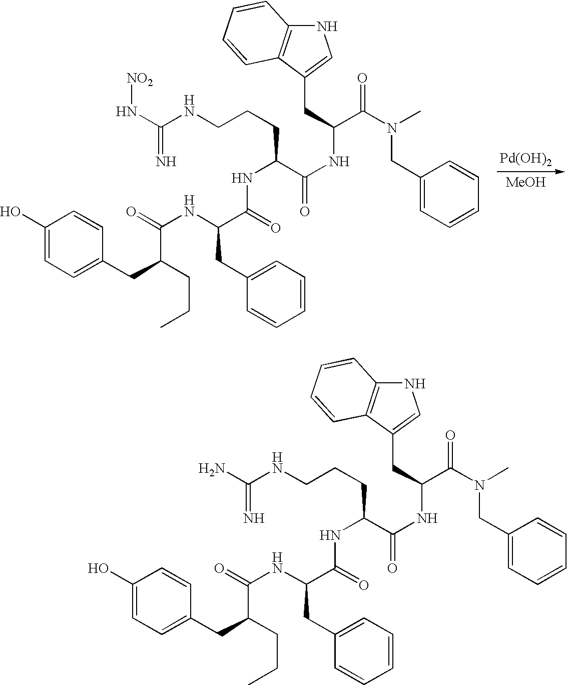

### EXAMPLE 99

### Synthesis of [2-(1-{1-[1-(Carbamoylmethyl-methyl-carbamoyl)-2-(1H-indol-3-yl)-ethylcarbamoyl]-4-guanidino-butylcarbamoyl}-2-phenyl-ethylcarbamoyl)-3-(4-hydroxy-phenyl)-propyl]-carbamic acid tert-butyl ester

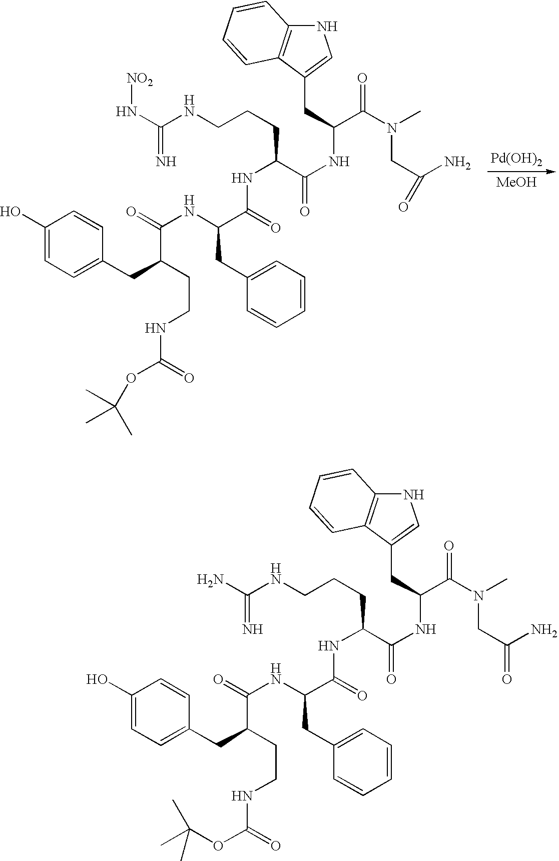

### C. Manual Solid Phase Chemistry

The following peptides are obtained by manual synthesis using Fmoc chemistry and Rink amide resin as the solid support. The removal of the Fmoc groups is achieved by reaction of 20% piperidine in DMF for 30 min followed by washing with DMF (3×35 ml), MeOH (3×35 ml), and DMF (3×35 ml.) The ninhydrin color test is used for monitoring reaction completion. Acetylation of the terminal amino group is done with 5% Ac2O/0.25% NMM/0.2% HOBt in DMF for 30 min followed by extensive washing with DMF and DCM and brief drying under vacuum. The crude product is cleaved from the resin and the protecting groups removed using 93% TFA and 2.3% ethanedithiol in water for 3 h at room temperature. After removal of the resin by filtration and washing with 3% TFA (3×18 ml), the filtrate is extracted with ether (6×20 ml) and is frozen and lyophilized. The crude product is then dissolved in 30% aqueous acetic acid and the solution purified by preparative HPLC (10μ Vydac C₁₈, 10×250 num, 6 to 60% acetonitrile-water (0.1% TFA) gradient over 1.5 h.) The product-containing fractions are is combined and lyophilized to give the purified peptides.

### EXAMPLE 100

### Synthesis of Ac—Y-(4-Py)ala-RW—NH2.2TFA

Fmoc-W(Boc)-OH and Fmoc-R(pbf)-OH (each in 2-fold excess) are attached sequentially to the Rink amide resin (4.28 g, 3 mmol) using PyBOP (2-fold excess) and NMM (4-fold excess). After washing with DMF (3×35 ml), ether (4×35 ml), and drying in vacuo an increase in weight is achieved. The resin (1.17 g, 0.35 mmol) is suspended in DMF (10 ml), the Fmoc group is removed, PyBOP (0.6 g, 1.15 mmol), NMM (0.26 ml, 2.8 mmol), and Fmoc-(4-Py)ala-OH (0.447 g, 1.15 mmol) are added sequentially, and the mixture is shaken for 1 h. Following Fmoc-deprotection, the coupling procedure is again repeated with Fmoc Y(t-Bu)-OH (0.528 g, 1.1 mmol). The peptide is then deprotected, acetylated, and cleaved from the resin to give of product.

### EXAMPLE 101

### Synthesis of Ac—Y-(3-Py)ala-RW—NH2.2TFA

Prepared according to Example 100, except Fmoc-(3-Py)ala-OH is used instead of Fmoc-(4-Py)ala-OH to yield the title compound.

### EXAMPLE 102

### Synthesis of Ac—Y-(2-Py)ala-RW—NH2.2TFA

Addition of Fmoc-(2-Py)ala-OH to a solution of PyBop and NMM in DMF leads to a very fast decomposition of the amino acid. Thus, this example is prepared using a modification of the procedure used for Example 100 in which NMM (88 ul, 0.8 mmol) is added to a solution of Fmoc-(2-Py)ala-OH (0.311 g, 0.8 mmol) and PyBrop (0.373 mg, 0.8 mmol). A second equivalent of NMM is then added after 15 min. After 100 min any un-reacted amino termini are acetylated and the remaining steps described in Example 100 are followed to give the title compound.

### VIII. Composition and Method Examples

### EXAMPLE A

An obese human female subject weighing 130 kg is treated by this method to incur weight loss. Specifically, once each day for a period of 6 months, the subject is administered, via intravenous injection, 15 ml of an aqueous solution comprising the following:

| Component | Concentration (mg/ml) |
|---|---|
| Compound of Ex. 1 | 5 |
| Sodium bisulfate | 1 |
| Sodium chloride | 7 |
| Chlorobutanol | 5 |
| Citric acid | 10 |
| Sterile water | qs to 1 mL |
| Sodium Hydroxide | adjust to pH 5 |

At the end of the treatment period, the patient exhibits measurable weight loss.

### EXAMPLE B

An obese human male subject weighing 150 kg is subjected to a weight-reduction program that achieves weight loss with reduced adiposity through a combination of a restricted diet and increased exercise. Specifically, once each day for a period of 6 months after weight loss, the subject is administered, via intravenous injection, 15 ml of an aqueous solution comprising the following:

| Component | Concentration (mg/ml) |
|---|---|
| Compound of Ex. 9 | 5 |
| Sodium bisulfate | 1 |
| Sodium chloride | 7 |
| Chlorobutanol | 5 |
| Citric acid | 10 |
| Sterile water | qs to 1 mL |
| Sodium Hydroxide | adjust to pH 5 |

At the end of the treatment period, the patient exhibits maintenance of weight loss and reduced adiposity.

### EXAMPLE C

An obese human male subject weighing 165 kg is subjected to a weight reduction program that achieves weight loss through a combination of restricted diet, increased exercise and subcutaneous injection daily of 15 mls of an aqueous solution comprising:

| Component | Concentration (mg/ml) |
|---|---|
| Compound of Ex. 88 | 5 |
| Sodium bisulfate | 1 |
| Sodium chloride | 7 |
| Chlorobutanol | 5 |
| Citric acid | 10 |
| Sterile water | qs to 1 mL |
| Sodium Hydroxide | adjust to pH 5 |

Once the desired weight loss has been achieved, the patient's weight loss is maintained through continuation of the intravenous injection once each day for an additional period of 6 months. At the end of the treatment period the patient exhibits maintained weight loss and reduced adiposity.

### EXAMPLE D

An obese human female subject weighing 140 kg is treated by the present method to incur weight loss. Specifically, she is treated with an implantable subcutaneous pump that delivers 0.1 mg/kg of the compound of Example 31 over a 24 hour period. The pump contains a solution of the compound dissolved in a solution of 50% propylene glycol and 50% sterile water. The pump is replaced monthly and treatment continues for a six-month period at which time the patient exhibits weight loss and reduced adiposity.

### EXAMPLE E

An obese male weighing 150 kg is treated by this method to incur weight loss. Specifically, he is treated with an oral tablet taken twice daily containing 300 mg of the compound in Example 29. The treatment continues for 12 months at which time the patient exhibits weight loss and reduced adiposity.

1. A compound having a structure according to Formula (I): 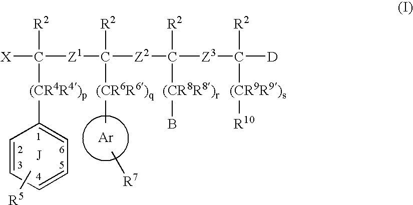
wherein
(A) X is selected from hydrogen, fluoro, aryloxy, acyloxy, OR¹, SR¹, —NR¹R^{1′} and —CHR¹R^{1′}, where R¹ and R^{1′} are independently selected from the group consisting of hydrogen, alkyl and acyl;
(B) (1) each R² is, independently selected from the group consisting of hydrogen, alkyl halo and heteroalkyl; or
(2)(a) two consecutive R² moieties, or consecutive R² and R³ moieties, may join to form a 3 to 8 membered carbocyclic or heterocyclic ring; or
(b) the R² bonded to the carbon atom that is bonded to X and Z¹ and an R⁵ moiety can optionally join to form a carbocyclic or heterocyclic ring that is fused to phenyl ring 3; or
(c) the R² bonded to the carbon atom that is bonded to ring Ar can join with an R⁷ to form a ring fused to ring Ar; or
(d) the R² bonded to the carbon atom that is bonded to Z² and Z³ can optionally join with R⁸ to form a carbocyclic or heterocyclic ring; or
(e) the R² bonded to the carbon atom that is bonded to Z³ and D can optionally join with R¹⁰ to form a carbocyclic or heterocyclic ring;
(C) each of Z¹, Z² and Z³ is independently selected from —OC(R³)(R^{3a})—; —C(R³)(R^{3a})O—; —S(O)ₐC(R³)(R^{3a})—, where a is 0, 1 or 2; —C(R³)(R^{3a})S(O)_{b}—, where b is 0, 1 or 2; —N(R^{3c})C(R³)(R^{3a})—; —C(R³)(R^{3a})N(R^{3c})—; —C(O)N(R^{3d})—; —N(R^{3d})C(O)—; —C(O)C(R³)(R^{3a})—; —C(R³)R^{3a})C(O)—; —C(R³)R^{3a}(C(R^{3b})(R^{3c})—; —C(R³)═C(R^{3a})—; —C≡C—; —SO₂N(R^{3d})—; —N(R^{3d})SO₂—; —C(R³)(R^{3a})P(═O)(OR^{3f})—; —P(═O)(OR^{3f})C(R³)(R^{3a})—; —N(R^{3d})P(═O)(OR^{3f})—; —P(═O)(O^{3f})N(R^{3d}d)—; —P(═O)(OR^{3f})O—; —O—P(═O)(OR^{3f})—; a cycloalkyl having from 3 to 8 ring atoms and a heterocycloalkyl having from 4 to 8 ring atoms; wherein
(1) each of R³, R^{3a} R^{3b} and R^{3c}, when present, is independently selected from hydrogen, hydroxy, alkoxy, aryloxy, acyloxy, thiol, alkylthio, acylthio, arylthio, amino, alkylamino, acylamino, and alkyl;
(2) R^{3d}, when present, is selected from hydrogen, alkyl and aryl;
(3) R^{3c}, when present, is selected from hydrogen, alkyl, aryl and acyl; and
(4) R^{3f}, when present, is selected from hydrogen and alkyl;
(D) p is 0, 1, 2, 3, 4 or S; wherein
(1) when p is greater than 0, each R⁴ and R^{4′} is independently selected from hydrogen, alkyl, aryl, halo, hydroxy, alkoxy, amino and acylamino;
(2) when p is greater than 1, two R⁴ moieties, together with the carbon atoms to which they are bonded, can join to form a heterocycloalkyl, cycloalkyl or aryl ring; and
(3) when p is greater than 1, the R⁴ moieties on two adjacent carbon atoms can both be nil such that a double bond is formed between the two adjacent carbon atoms, or both the R⁴ and R^{4′} moieties on two adjacent carbon atoms can all be nil such that a triple bond is formed between the two adjacent carbon atoms;
(E) R⁵ represents the 5 substituents (i.e., positions 2-6) on phenyl ring J, wherein each R⁵ is independently selected from hydrogen, hydroxy, halo, thiol, —OR¹², —SR¹², —SO₂N(R¹²)(R^{12′}), —N(R¹²)(R^{12′}), alkyl, acyl, alkene, alkyne, cyano, nitro, aryl, heteroaryl, cycloalkyl, and heterocycloalkyl; where each R¹² and R^{12′} is independently selected from hydrogen, alkyl, acyl, heteroalkyl, aryl, heteroaryl, cycloalkyl, and heterocycloalkyl; or two R⁵ moieties can optionally join to form a carbocyclic or a heterocyclic ring that is fused to phenyl ring J;
(F) q is 0, 1, 2, 3, 4 or 5; wherein
(1) when q is greater than 0, each R⁶ and R^{6′} is independently selected from hydrogen, alkyl, aryl, halo, hydroxy, alkoxy, amino and acylamino;
(2) when q is greater than 1, two R⁶ moieties, together with the carbon atoms to which they are bonded, can join to form a heterocycloalkyl, cycloalkyl or aryl ring; and
(3) when q is greater than 1, the R⁶ moieties on two adjacent carbon atoms can be nil such that a double bond is formed between the two adjacent carbon atoms, or both the R⁶ and R^{6′} moieties on two adjacent carbon atoms can all be nil such that a triple bond is formed between the two adjacent carbon atoms;
(G) Ar is an aryl or heteroaryl ring selected from the group consisting of phenyl, thiophene, furan, oxazole, thiazole, pyrrole and pyridine;
(H) R⁷ represents the substituents on ring Ar, wherein each R⁷ is independently selected from hydrogen, halo, —NR¹³R^{13′}, alkyl, acyl, alkene, alkyne, cyano, nitro, aryl, heteroaryl, cycloalkyl, and heterocycloalkyl; where each R¹³ and R^{13′} is independently selected from hydrogen, alkyl, acyl, heteroalkyl, aryl, heteroaryl, cycloalkyl, and heterocycloalkyl; or two R⁷ moieties can optionally join to form a carbocyclic or a heterocyclic ring fused to ring Ar;
(I) r is 0, 1, 2, 3, 4, 5, 6 or 7; wherein
(1) each R⁸ and R^{8′} is independently selected from hydrogen, alkyl, halo, hydroxy, alkoxy and amino;
(2) when r is greater than 1, two R⁸ moieties, together with the carbon atoms to which they arc bonded, can join to form a heterocycloalkyl, cycloalkyl or aryl ring; and
(3) when r is greater than 1, the R⁸ moieties on two adjacent carbon atoms can be nil such that a double bond is formed between the two adjacent carbon atoms, or both the R⁸ and R^{8′} moieties on two adjacent carbon atoms can all be nil such that a triple bond is formed between the two adjacent carbon atoms;
(J) B is selected from —N(R¹⁴)C(═NR¹⁵), ═O, or ═S)NR¹⁶R¹⁷, —NR²⁰R²¹, cyano (—CN), a heteroaryl ring eg. thiophene, an alkyl or dialkyl amine, a heteroaryl ring containing at least one ring nitrogen atom and a heterocycloalkyl ring containing a t least on e ring nitrogen atom, wherein R¹⁴, R¹⁵ R¹⁶, R¹⁷, R²⁰ and R²¹ are independently selected from hydrogen, alkyl, alkene, and alkyne; wherein further a combination of two or more of R¹⁴, R¹⁵, R¹⁶ and R¹⁷ may optionally combine with the atoms to which they are bonded to form a monocyclic or bicyclic ring; preferred are —N(R¹⁴)C(═NR¹⁵)NR¹⁶R¹⁷, cyano, N(R¹⁴)C(═O)NR¹⁶R¹⁷, a heteroaryl ring containing at least one ring nitrogen atom and a heterocycloalkyl ring containing at least one ring nitrogen atom. More preferred are —N(R¹⁴)C(═NR¹⁵)NR¹⁶R¹⁷, —N(R¹⁴)C(═O)NR¹⁶R¹⁷, cyano, and triazole and imidazole.
(K) s is 0, 1, 2, 3, 4 or 5; wherein
(1) when s is greater than 0, each R⁹ and R^{9′} is independently selected from hydrogen, alkyl, aryl, halo, hydroxy, alkoxy, amino and acylamino;
(2) when s is greater than 1, two R⁹ moieties, together with the carbon atoms to which they are bonded, can join to form a heterocycloalkyl, cycloalkyl or aryl ring; and
(3) when s is greater than 1, the R⁹ moieties on two adjacent carbon atoms can be nil such that a double bond is formed between the two adjacent carbon atoms, or both the R⁹ and R^{9′} moieties on two adjacent carbon atoms can all be nil such that a triple bond is formed between the two adjacent carbon atoms;
(L) R¹⁰ is selected from the group consisting of an optionally substituted bicyclic aryl ring and an optionally substituted bicyclic heteroaryl ring; and
(M) D is independently selected from hydrogen, fluoro, hydroxy, thiol, acylthio, alkoxy, aryloxy, alkylthio, acyloxy, cyano, amino, acylamino, —C(O)R¹¹ and —C(S)R¹¹; wherein R¹¹ is selected from the group consisting of hydroxy; alkoxy; amino; alkylamino; —NHOR¹⁸, where R¹⁸ is selected from hydrogen and alkyl, —N(R¹⁹)CH₂C(O)NH₂, where R¹⁹ is alkyl; —NHCH₂CH₂OH; —N(CH₃)CH₂CH₂OH; and —NHNHC(═Y)NH₂, where Y is selected from O, S and NH; and
(N) wherein if at least one of Z¹, Z² or Z³ is other than —C(O)N(R^{3d})— or —N(R^{3d})C(O)—, then X and D may optionally be linked together via a linking moiety, L, that contains all covalent bonds or covalent bonds and an ionic bond so as to form a cyclic peptide analog;
or an optical isomer, diastereomer or enantiomer thereof; a pharmaceutically-acceptable salt, hydrate, or biohydrolyzable ester, amide or imide thereof.

2. The compound according to claim 1 wherein X is selected from —NR¹R^{1′} and —CHR¹R^{1′} and wherein R¹ is hydrogen or alkyl and R^{1′} is acyl.

3. The compound according to claim 1 wherein each R² is hydrogen; or the R² bonded to the carbon atom bonded to Z³ and D joins with R¹⁰ to form a carbocyclic or heterocyclic ring and the other R² moieties are hydrogen.

4. The compound according to claim 1 wherein Z¹, Z² and Z³ are independently selected from are —OC(R³)(R^{3a})—; —C(R³)(R^{3a})O—; —C(R³)(R^{3a})N(R^{3c})—; —C(O)N(R^{3d})—; —C(R³)(R^{3a})C(R^{3b})(R^{3c}); —C(R³)═C(R^{3a})—; —SO₂N(R^{3d})— and —P(═O)(OR^{3f})C(R³)(R^{3a}).

5. The compound according to claim 4 wherein each R³, R^{3a}R^{3b} and R^{3c}, when present, is independently selected from hydrogen, hydroxy, alkoxy, aryloxy and alkyl; R^{3d}, when present, is selected from hydrogen and alkyl; R^{3c}, when present, is selected from hydrogen and alkyl; and when R^{3f} is present and is alkyl, said R^{3f} is branched alkyl.

6. The compound of claim 1 wherein p is 1 or 2.

7. The compound of claim 1 wherein each R⁴, when present, is hydrogen and each R^{4′}, when present, is hydrogen or alkyl.

8. The compound of claim 1 wherein each R⁵ is independently selected from hydrogen; hydroxy; halo; thiol; —SO₂N(R¹²)(R^{12′}) where R¹² and R^{12′} both are hydrogen; and —N(R¹²)(R^{12′}) where R¹² and R^{12′} each are hydrogen or alkyl.

9. The compound of claim 8 wherein four of the R⁵ moieties on ring J are hydrogen.

10. The compound of claim 9 wherein the 4-position of ring J is other than hydrogen.

11. The compound of claim 1 where q is 0, 1 or 2.

12. The compound of claim 1 wherein q is greater than 0, each R⁶ is hydrogen and each R^{6′} is hydrogen or alkyl.

13. The compound of claim 1 wherein Ar is selected from phenyl, thiophene and furan.

14. The compound of claim 13 wherein Ar is phenyl.

15. The compound of claim 14 where the 4-position of the phenyl ring is selected from hydrogen, fluoro, chloro, cyano, bromo, iodo, nitro and alkyl and the remaining four positions are hydrogen.

16. The compound of claim 1 wherein r is 2, 3 or 5 and each R⁸ and R^{8′} is independently selected from hydrogen and alkyl.

17. The compound of claim 1 wherein B is selected from —N(R¹⁴)C(═NR¹⁵)NR¹⁶R¹⁷, a heteroaryl ring containing at least one ring nitrogen atom and a heterocycloalkyl ring containing at least one ring nitrogen atom.

18. The compound of claim 17 wherein B is —N(R¹⁴)C(═NR¹⁵)NR¹⁶R¹⁷.

19. The compound of claim 18 wherein R¹⁴, R¹⁵, R¹⁶ and R¹⁷ are independently selected from hydrogen and alkyl.

20. The compound of claim 1 wherein s is 1 or 2 and R⁹ is hydrogen and each R^{9′} is hydrogen or alkyl.

21. The compound of claim 1 wherein R¹⁰ is selected from 1-naphthyl, 2-naphthyl, indan, 1H-indene, benzocylcobutane, benzocylcobutene, indole, indoline, pyrindine, dihydropyrindine, octahydropyrindine, benzothiophene, benzofuran, benzimidazole, benzopyran, quinoline, quinolone and isoquinoline.

22. The compound of claim 1 wherein D is selected from fluoro, hydroxy, thiol, alkoxy, aryloxy, alkylthio, acyloxy, cyano, amino, acylamino, —C(O)R¹¹ and —C(S)R¹¹.

23. The compound of claim 1 wherein X and D are linked together via a linking moiety, L, to provide a cyclic compound having a structure according to the following Formula (II):

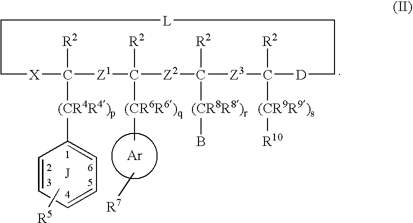

24. A compound having a structure according to Formula (A): 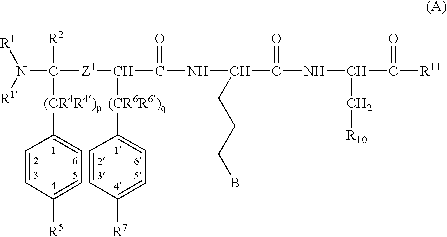
wherein
(A) R¹ and R^{1′} are independently selected from the group consisting of hydrogen, alkyl and acyl;
(B) R² is selected from the group consisting of hydrogen, alkyl and heteroalkyl;
(C) Z¹ is selected from —OC(R³)(R^{3a})—; —C(R³)(R^{3a})O—; —S(O)₂C(R³)(R^{3a})—; —C(R³)(R^{3a})S(O)₂—; —N(R^{3a})C(R³)(R^{3a})—; —C(R³)(R^{3a})N(R^{3c})—; —C(O)N(R^{3d})—; —N(R^{3d})C(O)—; —C(R³)(R^{3a})C(R^{3b})(R^{3c})—; —C(R³)═C(R^{3a})—; —C≡C—; —SO₂N(R^{3d})—; —N(R^{3d})SO₂—; —C(R³)(R^{3a})P(═O)(OR^{3f})—; and —P(═O)(OR^{3f})C(R³)(R^{3a})—; wherein
(1) each of R³, R^{3a}R^{3b} and R^{3c}, when present, is independently selected from hydrogen, hydroxy, alkoxy, aryloxy, acyloxy, thiol, alkylthio, acylthio, arylthio, amino, alkylamino, acylamino, and alkyl;
(2) R^{3d}, when present, is selected from hydrogen, alkyl and aryl;
(3) R^{3c}, when present, is selected from hydrogen, alkyl, aryl and acyl; and
(4) R^{3f}, when present, is selected from hydrogen and alkyl;
(D) p is 1 or 2 and each R⁴ and R^{4′} is independently selected from hydrogen, alkyl, aryl, halo, hydroxy, alkoxy, amino and acylamino;
(E) R⁵ is selected from hydrogen, hydroxy, chloro, fluoro, —N(R¹²)R^{12′}) where R¹² and R^{12′} each are independently selected from hydrogen and alkyl;
(F) q is 0, 1 or 2; wherein when q is greater than 0, each R⁶ and R⁶ is independently selected from hydrogen, alkyl, aryl, halo, hydroxy, alkoxy, amino and acylamino;
(G) R⁷ is selected from hydrogen, halo, —NR¹³R^{13′}, alkyl, acyl, alkene, alkyne, cyano, nitro, aryl, heteroaryl, cycloalkyl, and heterocycloalkyl; wherein each R¹³ and R^{13′} is independently selected from hydrogen and alkyl;
(H) B is selected from —N(R¹⁴)C(═NR¹⁵)NR¹⁶R¹⁷, a heteroaryl ring containing at least one ring nitrogen atom and a heterocycloalkyl ring containing at least one ring nitrogen atom; wherein R¹⁴, R¹⁵ R¹⁶ and R¹⁷ are independently selected from hydrogen and alkyl; wherein further a combination of two or more of R¹⁴, R¹⁵, R¹⁶ and R¹⁷ may optionally combine with the atoms to which they are bonded to form a monocyclic or bicyclic ring;
(I) R¹⁰ an optionally substituted bicyclic ring selected from 1-naphthyl, 2-naphthyl, indan, 1H-indent, benzocylcobutane, benzocylcobutene, indole, indoline, pyrindine, dihydropyrindine, octahydropyrindine, benzothiophene, benzofuran, benzimidozole, benzopyran, quinoline, quinolone and isoquinoline; and
(J) R¹¹ is selected from the group consisting of amino; alkylamino; —NHOR¹⁸, where R¹⁸ is selected from hydrogen and alkyl; —N(R¹⁹)CH₂C(O)NH₂, where R¹⁹ is alkyl; —NHCH₂CH₂OH; and —N(CH₃)CH₂CH₂OH,
or an optical isomer, diastereomer or enantiomer thereof; a pharmaceutically-acceptable salt, hydrate, or biohydrolyzable ester, amide or imide thereof.

25. A pharmaceutical composition comprising;
(a) a safe and effective amount of a cyclic peptide analog of claim 1; and
(b) a pharmaceutically-acceptable excipient.

26. A pharmaceutical composition comprising:
(a) a safe and effective amount of a cyclic peptide analog of claim 23; and
(b) a pharmaceutically-acceptable excipient.

27. A pharmaceutical composition comprising:
(a) a safe and effective amount of a cyclic peptide analog of claim 24; and
(b) a pharmaceutically-acceptable excipient.

28. A method of treating a disorder selected from the group consisting of insulin resistance, glucose intolerance, Type-2 diabetes mellitus, coronary artery disease, elevated blood pressure, hypertension, dyslipidaemia, cancer (e.g., endometrial, cervical, ovarian, breast, prostate, gallbladder, colon), menstrual irregularities, hirsutism, infertility, gallbladder disease, restrictive lung disease, sleep apnea, gout, osteoarthritis, and thromboembolic disease, in an animal subject, the method comprising administering to said subject the compound of claim 1.

29. A method of treating a disorder selected from the group consisting of a body weight disorder, CNS depression, behavior-related disorders, memory related disorders, cardiovascular function, inflammation, sepsis, septic shock, cardiogenic shock, hypovolemic shock, sexual dysfunction, erectile dysfunction, muscle atrophy, diseases associated with nerve growth and repair, and intrauterine fetal growth, in an animal subject, the method comprising administering to said subject the compound of claim 1.

30. The method of claim 29, wherein the disease is a body weight disorder selected from the group consisting of obesity, anorexia, and cachexia.

31. The method of claim 30, wherein the disease is obesity.
